(19) United States
(12) Patent Application Publication
(10) Pub. No.: US 2026/0049079 A1
(43) Pub. Date: Feb. 19, 2026

(54) CHIRAL SYNTHESIS OF FUSED BICYCLIC RAF INHIBITORS

(71) Applicant: Jazz Pharmaceuticals Ireland Limited, Dublin (IE)

(72) Inventors: Andrew BELFIELD, Macclesfield (GB); Neil Hawkins, Macclesfield (GB); Steven Christopher Glossop, Macclesfield (GB); Jean-François Margathe, Macclesfield (GB); Clifford David Jones, Macclesfield (GB); Chiara Colletto, Macclesfield (GB)

(21) Appl. No.: 19/268,592

(22) Filed: Jul. 14, 2025

Related U.S. Application Data
(63) Continuation of application No. 17/387,041, filed on Jul. 28, 2021, now abandoned.
(60) Provisional application No. 63/057,531, filed on Jul. 28, 2020.

(51) Int. Cl. C07D 471/04; A61K 45/06; B01J 31/22; C07D 405/14

(52) U.S. Cl. CPC C07D 471/04; A61K 45/06; B01J 31/22; C07D 405/14; B01J 2531/821

(57) ABSTRACT

The present disclosure generally relates to improved synthesis of fused bicyclic Raf inhibitor enantiomers of formula (I), (Ia), (Ib), (II), (IIa), or (IIb), or a pharmaceutically acceptable salt, tautomer, or stereoisomer thereof, with high enantiomeric excess (% ee). The disclosure also relates to method of using the compound of formula (I), (Ia), (Ib), (II), (IIa), or (IIb), or a pharmaceutically acceptable salt, tautomer, or stereoisomer thereof, for treating diseases such as cancer, including colorectal cancer.

### CROSS-REFERENCE TO RELATED APPLICATIONS

This application is a continuation of U.S. Utility application Ser. No. 17/387,041, filed Jul. 28, 2021, which claims the benefit of U.S. Provisional Application No. 63/057,531, filed Jul. 28, 2020, the disclosure of each of which is incorporated by reference herein in its entirety.

### FIELD OF THE INVENTION

The present disclosure generally relates to improved synthesis of fused bicyclic Raf inhibitor enantiomers with high enantiomeric excess (% ee).

### BACKGROUND OF THE INVENTION

Mutations leading to uncontrolled signaling via the RAS-RAF-MAPK pathway are seen in more than one third of all cancers. The RAF kinases (A-RAF, B-RAF and C-RAF) are an integral part of this pathway, with B-RAF mutations commonly seen in the clinic. Although most B-RAF V600E mutant skin cancers are sensitive to approved B-RAF selective drugs, B-RAF V600E mutant colorectal cancers are surprisingly insensitive to these agents as monotherapy due to the functions of other RAF family members and require combination therapy. B-RAF selective therapies fail to show clinical benefit against atypical B-RAF (non-V600E), other RAF and RAS driven tumors.

U.S. Pat. No. 10,183,939 discloses racemic Raf inhibitors that demonstrated binding affinity for B-RAF V600E and C-RAF, the disclosure of which is hereby incorporated by reference in its entirety. These pan-RAF inhibitors are identified to be promising candidates in overcome resistance mechanisms associated with clinically approved B-RAF selective drugs. However, methods for selectively synthesizing enantiomers of the Raf inhibitors was not described in U.S. Pat. No. 10,183,939.

### SUMMARY OF THE INVENTION

The present disclosure relates to a method of synthesizing a compound of formula (Ia), or (Ib), or a pharmaceutically acceptable salt or tautomer thereof,

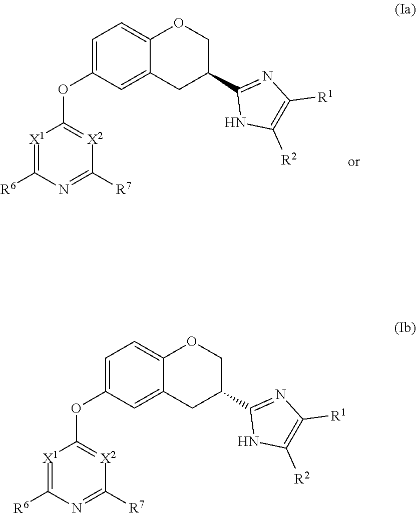

wherein:
R¹ is selected from substituted or unsubstituted: C₁₋₆ alkyl, C₁₋₆ haloalkyl, aryl, heterocyclyl, or heteroaryl;
R² is H,
X¹ is N or CR⁸;
X² is N or CR⁹;
R⁶ is hydrogen, halogen, alkyl, alkoxy, —NH₂, —NR^{F}C(O)R⁵, —NR^{F}C(O)CH₂R⁵, —NR^{F}C(O)CH(CH₃)R⁵, or —NR^{F}R⁵;
R⁷, R⁸, and R⁹ are each independently, hydrogen, halogen, or alkyl;
or alternatively, R⁶ and R⁸ together or R⁷ and R⁹ together with the atoms to which they are attached forms a 5- or 6-membered partially unsaturated or unsaturated ring containing 0, 1, or 2 heteroatoms selected from N, O, or S, wherein the ring is substituted or unsubstituted;
R⁵ is substituted or unsubstituted group selected from alkyl, carbocyclyl, aryl, heterocyclyl, or heteroaryl; and
R^{F} is selected from H or C₁₋₃ alkyl.

the method comprising:
a) reacting a compound of formula 1A with (R)-6-hydroxychromane-3-carboxylic acid or (S)-6-hydroxychromane-3-carboxylic acid to provide compound 2A;
wherein the compound of formula 2A has an (R) or (S) stereochemistry at the carbon indicated by *;

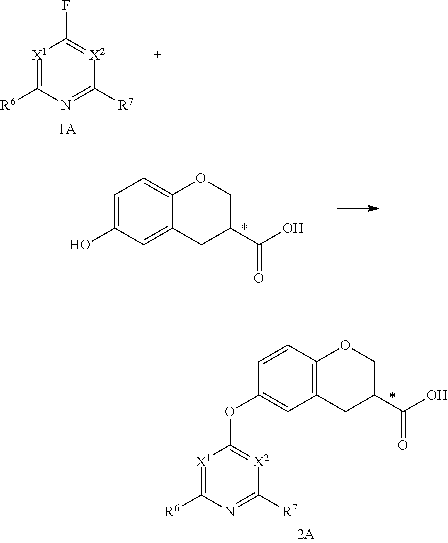

b) reacting compound 2A with a compound of formula 3A, or a salt thereof, to provide a compound of formula 4A;
wherein the compound of formula 4A has an (R) or (S) stereochemistry at the carbon indicated by *; and

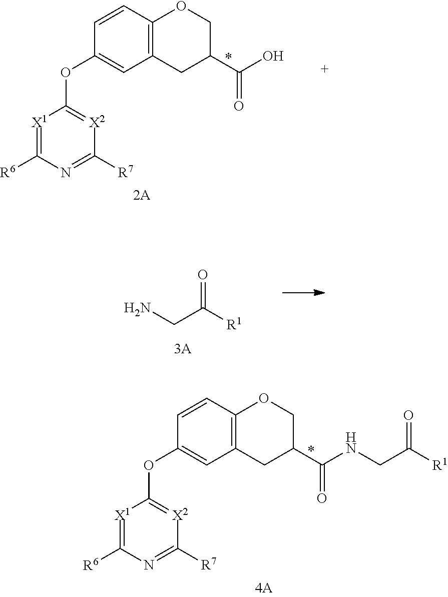

c) cyclizing the compound of formula 4A of step b) in the presence of ammonia or an ammonium salt to provide the compound of formula (Ia) or (Ib), or a pharmaceutically acceptable salt or tautomer thereof.

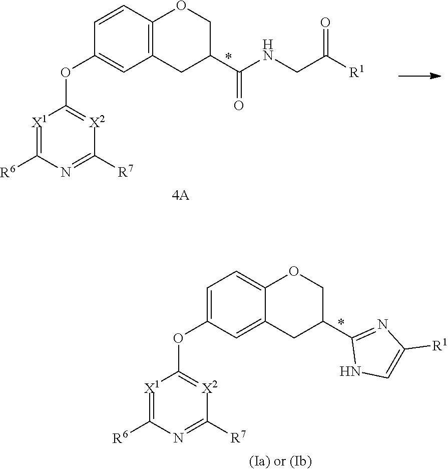

The present disclosure relates to a method of synthesizing a compound of formula (IIa), or (IIb), or a pharmaceutically acceptable salt or tautomer thereof,

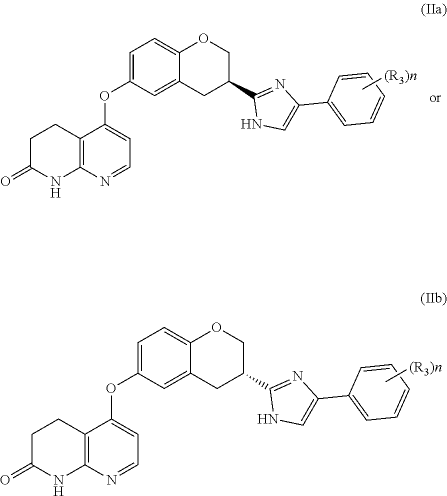

wherein:
R³ is halogen, —OR^{A}, —NR^{A}R^{B}, —SO₂R^{C}, —SOR^{C}, —CN, C₁₋₄ alkyl, C₁₋₄ haloalkyl, or C₃₋₆ cycloalkyl, wherein the alkyl, haloalkyl and cycloalkyl groups are optionally substituted with 1 to 3 groups independently selected from: —OR^{A}, —CN, —SOR^{C}, or —NR^{A}R^{B},
R^{A} and R^{B} are each independently selected from H, C₁₋₄ alkyl and C₁₋₄ haloalkyl;
R^{C} is selected from C₁₋₄ alkyl and C₁₋₄ haloalkyl; and
n is 0, 1, 2, 3, or 4;

the method comprising:
a) reacting 5-fluoro-3,4-dihydro-1,8-naphthyridin-2 (1H)-one with (R)-6-hydroxychromane-3-carboxylic acid or (S)-6-hydroxychromane-3-carboxylic acid to provide (R)-6-((7-oxo-5,6,7,8-tetrahydro-1,8-naphthyridin-4-yl)oxy) chromane-3-carboxylic acid or (S)-6-((7-oxo-5,6,7,8-tetrahydro-1,8-naphthyridin-4-yl)oxy) chromane-3-carboxylic acid;

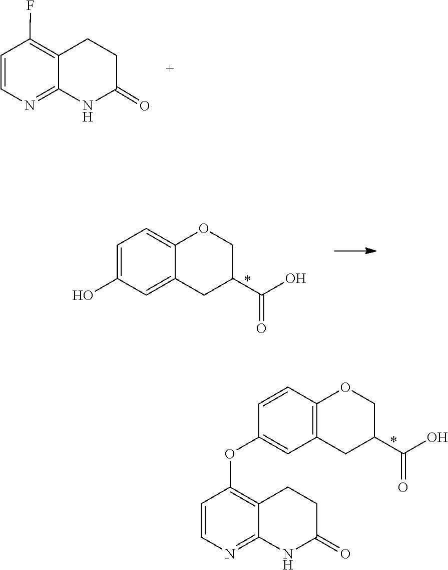 [SMILES: O=C1CCc2c(F)ccnc2N1 ; O=C(O)C1COc2ccc(O)cc2C1 ; O=C1CCc2c(Oc3ccc4c(c3)CC(C(=O)O)CO4)ccnc2N1]

b) reacting (R)-6-((7-oxo-5,6,7,8-tetrahydro-1,8-naphthyridin-4-yl)oxy) chromane-3-carboxylic acid or (S)-6-((7-oxo-5,6,7,8-tetrahydro-1,8-naphthyridin-4-yl)oxy) chromane-3-carboxylic acid with a 2-amino-1-phenylethan-1-one or a pharmaceutically acceptable salt thereof, to provide a compound of formula 4B,
wherein the 2-amino-1-phenylethan-1-one is optionally substituted with R³; and
wherein the compound of formula 4B has an (R) or (S) stereochemistry at the carbon indicated by *; and

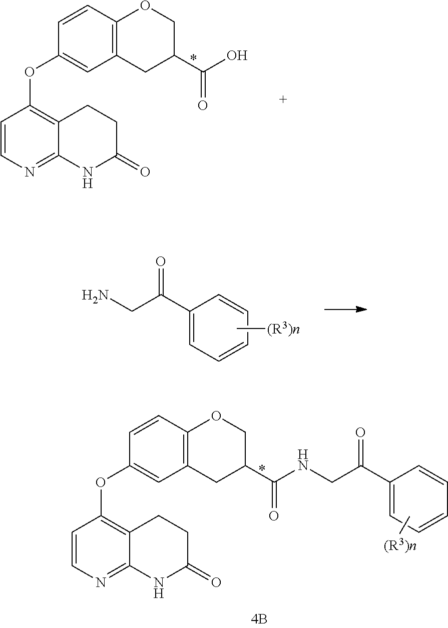

c) cyclizing the compound of formula 4B of step b) in the presence of ammonia or an ammonium salt to provide the compound of formula (IIa), or (IIb), or a pharmaceutically acceptable salt or tautomer thereof.

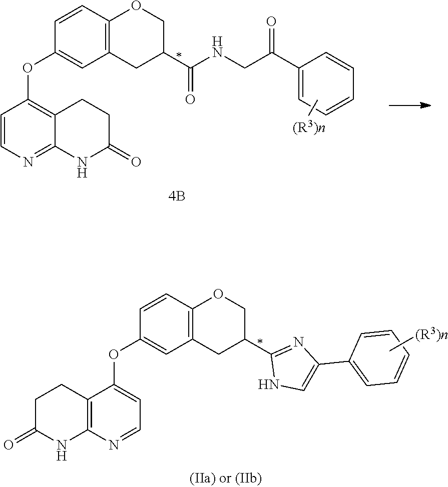

In embodiments of the synthetic methods disclosed herein, (R)-6-hydroxychromane-3-carboxylic acid or (S)-6-hydroxychromane-3-carboxylic acid is prepared by chiral hydrogenation of 6-hydroxy-2H-chromene-3-carboxylic acid.

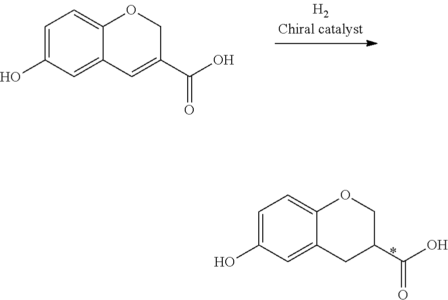 [SMILES: O=C(O)C1=Cc2cc(O)ccc2OC1 ; O=C(O)C1COc2ccc(O)cc2C1]

In embodiments of the synthetic methods disclosed herein, the chiral hydrogenation is performed in the presence of Ru or Rh catalyst and a chiral ligand. In embodiments, Ru or Rh catalyst is selected from Ru(OAc)₂, [RuCl₂(p-cym)]₂, Ru(COD)(Me-allyl)₂, Ru(COD)(TFA)₂, [Rh(COD)₂]OTf or [Rh(COD)₂]BF₄. In embodiments, the Ru catalyst is selected from [RuCl₂(p-cym)]₂, Ru(COD)(Me-allyl)₂, or Ru(COD)(TFA)₂. In embodiments, the chiral ligand is selected from (S)- or (R)-BINAP, (S)- or (R)-H8-BINAP, (S)- or (R)-PPhos, (S)- or (R)-Xyl-PPhos, (S)- or (R)-PhanePhos, (S)- or (R)-Xyl-PhanePhos, (S,S)-Me-DuPhos, (R,R)-Me-DuPhos, (S,S)-iPr-DuPhos, (R,R)-iPr-DuPhos, (S,S)-NorPhos, (R,R)-NorPhos, (S,S)-BPPM, or (R,R)-BPPM, or Josiphos SL-J002-1. In embodiments, the chiral ligand is selected from (S)- or (R)-PhanePhos or (S) or (R)-An-PhanePhos.

In embodiments of the synthetic methods disclosed herein, the chiral hydrogenation is performed in the presence of a chiral Ru-complex or a chiral Rh-complex. In embodiments, the chiral Ru-complex or the chiral Rh-complex is selected from [(R)-Phanephos-RuCl₂(p-cym)], [(S)-Phanephos-RuCl₂(p-cym)], [(R)-An-Phanephos-RuCl₂(p-cym)], [(S)-An-Phanephos-RuCl₂(p-cym)], [(R)-BINAP-RuCl(p-cym)]Cl, [(S)-BINAP-RuCl(p-cym)]Cl, (R)-BINAP-Ru(OAc)₂, (S)-BINAP-Ru(OAc)₂, [(R)-Phanephos-Rh(COD)]BF₄, [(S)-Phanephos-Rh(COD)]BF₄, [(R)-Phanephos-Rh(COD)]OTf, or [(S)-Phanephos-Rh(COD)]OTf. In embodiments, the chiral Ru-complex is selected from [(R)-Phanephos-RuCl₂(p-cym)], [(S)-Phanephos-RuCl₂(p-cym)], [(R)-An-Phanephos-RuCl₂(p-cym)], or [(S)-An-Phanephos-RuCl₂(p-cym)].

In embodiments of the synthetic methods disclosed herein, the chiral hydrogenation is performed with a substrate/catalyst loading in the range of about 25/1 to about 1,000/1. In embodiments, the substrate/catalyst loading in the range of about 200/1 to about 1,000/1.

In embodiments of the synthetic methods disclosed herein, the chiral hydrogenation is performed in the presence of a base. In embodiments, the base is triethylamine, NaOMe or Na₂CO₃. In embodiments, the base is used in about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 equivalent with respect to 6-hydroxy-2H-chromene-3-carboxylic acid.

In embodiments of the synthetic methods disclosed herein, the chiral hydrogenation is performed at a temperature in the range of about 30° C. to about 50° C.

In embodiments of the synthetic methods disclosed herein, the chiral hydrogenation is performed at a concentration of 6-hydroxy-2H-chromene-3-carboxylic acid in the range of about 0.2M to about 0.8M.

In embodiments of the synthetic methods disclosed herein, the chiral hydrogenation is performed at hydrogen pressure in the range of about 2 bar to about 30 bar. In embodiments, the hydrogen pressure in the range of about 3 bar to about 10 bar.

In embodiments of the synthetic methods disclosed herein, the chiral hydrogenation is performed in an alcohol solvent. In embodiments, the solvent is methanol, ethanol, or isopropanol.

In embodiments of the synthetic methods disclosed herein, (R)-6-hydroxychromane-3-carboxylic acid and (S)-6-hydroxychromane-3-carboxylic acid has an enantiomeric excess of at least 90%.

In embodiments of the synthetic methods disclosed herein, (R)-6-((7-oxo-5,6,7,8-tetrahydro-1,8-naphthyridin-4-yl)oxy) chromane-3-carboxylic acid and (S)-6-((7-oxo-5,6,7,8-tetrahydro-1,8-naphthyridin-4-yl)oxy) chromane-3-carboxylic acid has an enantiomeric excess of at least 90%.

In embodiments of the synthetic methods disclosed herein, the compound of formula 4A of step b) has an enantiomeric excess of at least 90%.

In embodiments of the synthetic methods disclosed herein, the compound of formula 4B of step b) has an enantiomeric excess of at least 90%.

In embodiments of the synthetic methods disclosed herein, the compound of formula (IIa) and (IIb), or a pharmaceutically acceptable salt or tautomer thereof, has an enantiomeric excess of at least 90%.

In embodiments of the synthetic methods disclosed herein, the compound of formula (Ia) and (Ib), or a pharmaceutically acceptable salt or tautomer thereof, has an enantiomeric excess of at least 90%.

In embodiments of the synthetic methods disclosed herein, R³ in formula (IIa) or (IIb) is halogen, C₁₋₄ alkyl, —SO₂ (C₁₋₄ alkyl). In embodiments, R³ is F, Cl, Br, or I. In embodiments, n is 0, 1, or 2.

In embodiments of the synthetic methods disclosed herein, R¹ in formula (Ia) or (Ib) is substituted or unsubstituted heteroaryl.

In embodiments of the synthetic methods disclosed herein, the compound is selected from

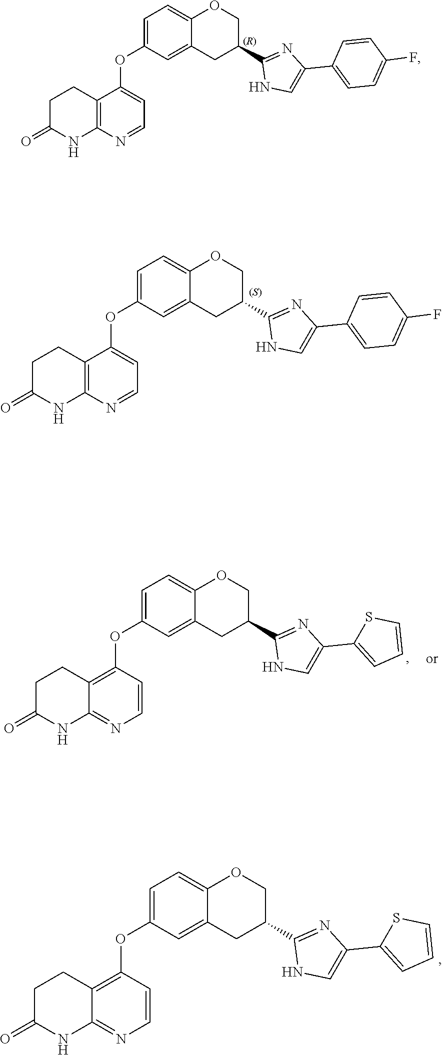

or a pharmaceutically acceptable salt or tautomer thereof. In embodiments of the synthetic methods disclosed herein, the compound is selected from Compounds A-1-N-1 or A-2-N-2, or a pharmaceutically acceptable salt or tautomer thereof, prepared by any of the methods as disclosed herein.

The present disclosure relates to a compound of formula (IIa), or (IIb), or a pharmaceutically acceptable salt or tautomer thereof, prepared by any of the methods as disclosed herein.

The present disclosure relates to a compound of formula (Ia), or (Ib), or a pharmaceutically acceptable salt or tautomer thereof, prepared by any of the methods as disclosed herein.

The present disclosure relates to Compounds A-1-N-1 or A-2-N-2, or a pharmaceutically acceptable salt or tautomer thereof, prepared by any of the methods as disclosed herein.

The present disclosure relates to Compounds A-1-N-1 or A-2-N-2, or a pharmaceutically acceptable salt or tautomer thereof.

In embodiments of the compounds of the disclosure, the compound has an enantiomeric excess of at least 90%. In embodiments, the compound has an enantiomeric excess of at least 95%. In embodiments, the compound has a chemical purity of 85% or greater. In embodiments, the compound has a chemical purity of 90% or greater. In embodiments, the compound has a chemical purity of 95% or greater.

The present disclosure relates to a pharmaceutical composition comprising any one of the compounds as disclosed herein and a pharmaceutically acceptable excipient or carrier.

In embodiments of the pharmaceutical composition, the composition further comprises an additional therapeutic agent. In embodiments, the additional therapeutic agent is selected from an antiproliferative or an antineoplastic drug, a cytostatic agent, an anti-invasion agent, an inhibitor of growth factor function, an antiangiogenic agent, a steroid, a targeted therapy agent, or an immunotherapeutic agent.

The present disclosure relates to a method of treating a condition which is modulated by a RAF kinase, comprising administering an effective amount of any one of the compounds disclosed herein.

In embodiments of the method of treatment, the condition treatable by the inhibition of one or more Raf kinases. In embodiments, the condition is selected from cancer, sarcoma, melanoma, skin cancer, haematological tumors, lymphoma, carcinoma or leukemia. In embodiments, the condition is selected from Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancers.

The present disclosure relates to a method of treating cancer, comprising administering an effective amount of any one of the compounds disclosed herein.

In embodiments of the method of treating cancer, the cancer comprises at least one mutation of the BRAF kinase. In embodiments, the cancer comprises a BRAF^{V600E} mutation.

In embodiments, the cancer is selected from melanomas, thyroid cancer, Barret's adenocarcinoma, biliary tract carcinomas, breast cancer, cervical cancer, cholangiocarcinoma, central nervous system tumors, glioblastomas, astrocytomas, ependymomas, colorectal cancer, large intestine colon cancer, gastric cancer, carcinoma of the head and neck, hematologic cancers, leukemia, acute lymphoblastic leukemia, myelodysplastic syndromes, chronic myelogenous leukemia, Hodgkin's lymphoma, non-Hodgkin's lymphoma, megakaryoblastic leukemia, multiple myeloma, hepatocellular carcinoma, lung cancer, ovarian cancer, pancreatic cancer, pituitary adenoma, prostate cancer, renal cancer, sarcoma, uveal melanoma or skin cancer. In embodiments, the cancer is BRAF^{V600E} melanoma, BRAF^{V600E} colorectal cancer, BRAF^{V600E} papillary thyroid cancers, BRAF^{V600E} low grade serous ovarian cancers, BRAF^{V600E} glioma, BRAF^{V600E} hepatobiliary cancers, BRAF^{V600E} hairy cell leukemia, BRAF^{V600E} non-small cell cancer, or BRAF^{V600E} pilocytic astrocytoma. In embodiments, the cancer is colorectal cancer.

### BRIEF DESCRIPTION OF THE DRAWINGS

FIG. 1 shows results with [(S)-BINAP-RuCl(p-cym)]Cl catalyst at different temperatures and substrate concentrations for reaction of compound 1 to P1 and/or P2. (Example 1, part C).

FIG. 2 shows hydrogen uptakes records from the Endeavor software for reactions disclosed in Table 10.

FIG. 3A shows overlay of hydrogen uptake records from Endeavor software for hydrogenation reaction with different substrate concentration as disclosed in Table 11, entries 1-2). FIG. 3B shows FIG. 3A hydrogen uptake records where the line for the lower substrate concentration (Table 11, entry 2) was shifted in time (to the right) so that the first data point lined up with the higher substrate concentration reaction.

FIG. 3C shows overlay of hydrogen uptake records from reactions disclosed in Table 11, entries 1-3, where the lines corresponding to entries 1 and 2 were shifted in time so that the first data point lined up with the higher substrate concentration reaction.

FIG. 3D shows overlay of hydrogen uptake records from reactions disclosed in Table 11, entries 1 and 4, where the lines corresponding to entry 4 was shifted in time so that the first data point lined up with the higher substrate concentration reaction.

FIG. 4 shows comparison of the rate of reaction for the reaction carried out in the Parr vessel (larger scale) with the reaction in the Endeavor (small scale), based on hydrogen uptake records.

FIG. 5 shows comparison of the rate of reaction for the reaction carried out in the Parr vessel (larger scale) with the reaction in the Endeavor (small scale), based on hydrogen uptake records.

FIG. 6 shows comparison of the rate of reaction with different catalyst loading (S/C 1,000/1 vs S/C 200/1), based on hydrogen uptake records.

FIG. 7 shows chiral LCMS chromatogram of Compound A-1 and Compound A-2.

FIG. 8A shows Ortep image of Compound P2 single crystal obtained in acetonitrile by slow evaporation. FIG. 8B shows Ortep image of Compound P2 single crystal obtained in THF/water by slow evaporation.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a Raf inhibitor” refers to one or more Raf inhibitor or at least one Raf inhibitor. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The compounds of the invention, or their pharmaceutically acceptable salts contain at least one asymmetric center. The compounds of the invention with one asymmetric center give rise to enantiomers, where the absolute stereochemistry can be expressed as (R)- and (S)-, or (+) and (−). When the compounds of the invention have more than two asymmetric centers, then the compounds can exist as diastereomers or other stereoisomeric forms. The present disclosure is meant to include all such possible isomers, as well as their racemic and optically pure forms whether or not they are specifically depicted herein. Optically active (+) and (−) or (R)- and (S)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present disclosure includes tautomers of any said compounds.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. The subject can be suspected of having or at risk for having a cancer, including but not limited to colorectal cancer and melanoma.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The term “halo” refers to a halogen. In particular the term refers to fluorine, chlorine, bromine and iodine.

“Alkyl” or “alkyl group” refers to a fully saturated, straight or branched hydrocarbon chain group, and which is attached to the rest of the molecule by a single bond. Alkyls comprising any number of carbon atoms, including but not limited to from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₈alkyl. A C₁-C₈alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, 1-butyl, n-pentyl, 1-amyl, n-hexyl, n-heptyl, n-octyl, n-Nonyl, n-decyl, n-undecyl, and n-dodecyl. Unless stated otherwise specifically in the specification, an alkyl group can be optionally substituted.

“Cycloalkyl” refers to a stable non-aromatic monocyclic or polycyclic fully saturated hydrocarbon group consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl groups include, for example, cyclopropyl, cyclobutyl, cyclopentyl, cyclohexyl, cycloheptyl, and cyclooctyl. Polycyclic cycloalkyl groups include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Haloalkyl” refers to an alkyl group, as defined above, that is substituted by one or more halo groups, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Aryl” refers to a hydrocarbon ring system group comprising hydrogen, 6 to 18 carbon atoms and at least one aromatic ring. For purposes of this invention, the aryl group can be a monocyclic, bicyclic, tricyclic or tetracyclic ring system, which can include fused or bridged ring systems. Aryl groups include, but are not limited to, aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl groups that are optionally substituted.

“Heterocyclyl,” “heterocyclic ring” or “heterocycle” refers to a stable 3- to 20-membered ring group which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl group can be a monocyclic, bicyclic, tricyclic or tetracyclic ring system, which can include fused or bridged ring systems; and the nitrogen, carbon or sulfur atoms in the heterocyclyl group can be optionally oxidized; the nitrogen atom can be optionally quaternized; and the heterocyclyl group can be partially or fully saturated. Examples of such heterocyclyl groups include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted. In embodiments, heterocyclyl, heterocyclic ring or heterocycle is a stable 3- to 20-membered non-aromatic ring group which consists of two to twelve carbon atoms and from one to six heteroatoms selected from the group consisting of nitrogen, oxygen and sulfur.

“Heteroaryl” refers to a 5- to 20-membered ring system group comprising hydrogen atoms, one to thirteen carbon atoms, one to six heteroatoms selected from the group consisting of nitrogen, oxygen and sulfur, and at least one aromatic ring. For purposes of this invention, the heteroaryl group can be a monocyclic, bicyclic, tricyclic or tetracyclic ring system, which can include fused or bridged ring systems; and the nitrogen, carbon or sulfur atoms in the heteroaryl group can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing groups can also be optionally substituted with one or more of the above groups.

### Compounds of the Invention

The present disclosure relates to pan-RAF inhibitors having the structure of formula (I), or a pharmaceutically acceptable salt, tautomer, or stereoisomer thereof,

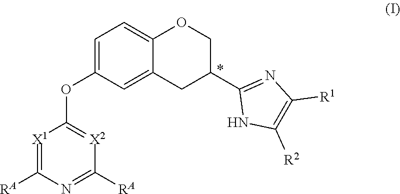

wherein one of R¹ or R² is selected from substituted or unsubstituted. C₁₋₆ alkyl, C₁₋₆ haloalkyl, aryl, heterocyclyl, or heteroaryl, and the other R¹ or R² is H;
or alternatively, R¹ and R² together with the atoms to which they are attached forms a 5- or 6-membered partially unsaturated or unsaturated ring containing 0, 1, or 2 heteroatoms selected from N, O, or S;
X¹ is N or CR^{AA};
X² is N or CR^{BB},
R⁶ is hydrogen, halogen, alkyl, alkoxy, —NH₂, —NR^{F}C(O)R⁵, —NR^{F}C(O)CH₂R⁵, —NRC(O)CH(CH₃)R⁵, or —NR^{F}R⁵;
R⁷, R⁸, and R⁹ are each independently, hydrogen, halogen, or alkyl;
or alternatively, R⁶ and R⁸ together or R⁷ and R⁹ together with the atoms to which they are attached forms a 5- or 6-membered partially unsaturated or unsaturated ring containing 0, 1, or 2 heteroatoms selected from N, O, or S, wherein the ring is substituted or unsubstituted;
R⁵ is substituted or unsubstituted group selected from alkyl, carbocyclyl, aryl, heterocyclyl, or heteroaryl; and
R^{F} is selected from H or C₁₋₃ alkyl.

In embodiments, the compounds of the formula (I) has the following stereochemistry.

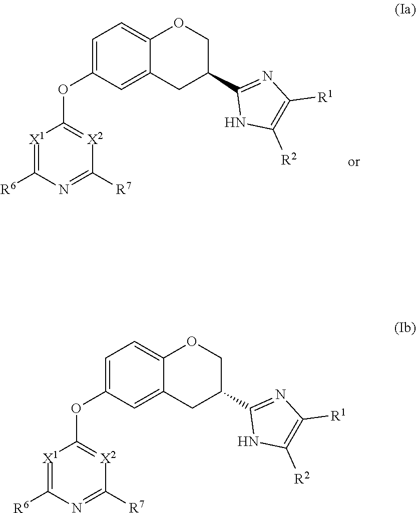

In embodiments, the compounds of the formula (I) has the stereochemistry as shown in formula (Ib).

In embodiments of the compounds of formula (I), R¹ and R² is substituted with halo, —OR^{A}, —NR^{A}R^{B}, —SO₂R^{C}, —SOR^{C}, —CN, C₁₋₄ alkyl, C₁₋₄ haloalkyl, or C₃₋₆ cycloalkyl, wherein the alkyl, haloalkyl and cycloalkyl groups are optionally substituted with 1 to 3 groups independently selected from: —OR^{A}, —CN, —SOR^{C}, or —NR^{A}R^{B},
wherein R^{A} and R^{B} are each independently selected from H, C₁₋₄alkyl and C₁₋₄ haloalkyl; and
wherein R^{C} is selected from C₁₋₄ alkyl and C₁₋₄ haloalkyl.

In embodiments of the compounds of formula (I), (Ia), or (Ib), one of R¹ or R² is selected from substituted or unsubstituted: phenyl, 5- or 6-membered heteroaryl containing 1 or 2 heteroatoms selected from N, O, or S, or a fused bicycle having 8, 9, or 10 ring members. In embodiments of the compounds of formula (I), (Ia), or (Ib), one of R¹ or R² is phenyl or 5,6-membered heteroaryl containing 1 or 2 heteroatoms. In embodiments of the compounds of formula (I), (Ia), or (Ib), one of R¹ or R² is phenyl, pyridyl, imidazole, pyrazole, thiophene,

In embodiments of the compounds of formula (I), (Ia), or (Ib), one of R¹ or R² is a fused bicycle having 8, 9, or 10 ring members, wherein 0, 1, 2, or 3, ring atoms are heteroatoms selected from N, O, or S. In embodiments of the compounds of formula (I), (Ia), or (Ib), one of R¹ or R² is a fused bicycle having 8, 9, or 10 ring members, wherein 0, 1, 2, or 3, ring atoms are heteroatoms selected from N, O, or S. and wherein both fused rings are aromatic rings or one ring is aromatic and the other ring is non-aromatic.

In embodiments of the compounds of formula (I), (Ia), or (Ib), R¹ and R² together forms a phenyl ring (makes benzoimidazole with the imidazole ring drawn in formula (I)), which is optionally substituted. In embodiments of the compounds of formula (I), (Ia), or (Ib), R′ and R² together forms a 5, or 6-membered ring containing one heteroatom selected from N, S, or O, which is optionally substituted.

In embodiments of the compounds of formula (I), (Ia), or (Ib), R⁶ and R⁸ together with the atoms to which they are attached forms a 5- or 6-membered partially unsaturated or unsaturated ring containing 0, 1, or 2 heteroatoms selected from N, O, or S, wherein the ring is substituted or unsubstituted. In embodiments, R⁷ and R⁹ together with the atoms to which they are attached forms a 5- or 6-membered partially unsaturated or unsaturated ring containing 0, 1, or 2 heteroatoms selected from N, O, or S, wherein the ring is substituted or unsubstituted.

In embodiments of the compounds of formula (I), (Ia), or (Ib), R⁶ and R⁸ together with the atoms to which they are attached forms a 5- or 6-membered partially unsaturated or unsaturated ring containing 1 or 2 heteroatoms selected from N, O, or S, wherein the ring is substituted or unsubstituted. In embodiments of the compounds of formula (I), (Ia), or (Ib), R⁶ and R⁸ together with the atoms to which they are attached forms a 5- or 6-membered partially unsaturated or unsaturated ring containing a nitrogen atom as a ring member, wherein the ring is substituted or unsubstituted. In embodiments, the ring is substituted with oxo. In embodiments, R⁷ and R⁹ are both hydrogen.

In embodiments of the compounds of formula (I), (Ia), or (Ib), R⁶ and R⁸ together with the ring to which they are attached forms

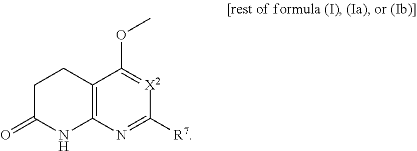

In embodiments, X² is CH; R⁷ is H; and R⁶ and R⁸ together with the ring to which they are attached forms

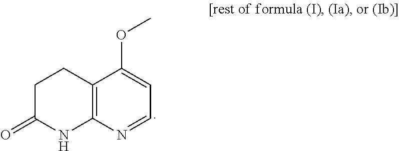

In embodiments of the compounds of formula (I), (Ia), or (Ib), R⁶ is halogen or C₁-C₃ alkyl. In embodiments of the compounds of formula (I), (Ia), or (Ib), R⁶ is —NHC(O)R⁵, —NHC(O)CH₂R⁵, —NHC(O)CH(CH₃)R⁵, or —NHR⁵.

In embodiments of the compounds of formula (I), (Ia), or (Ib), R⁷, R⁸, and R⁹ are each independently, hydrogen or methyl. In embodiments of the compounds of formula (I), (Ia), or (Ib), R⁷, R⁸, and R⁹ are each independently, hydrogen.

In embodiments of the compounds of formula (I), (Ia), or (Ib), R⁵ is substituted or unsubstituted group selected from alkyl, 3-6 membered carbocyclyl, phenyl, 3-6 membered heterocyclyl, or 5-6 membered heteroaryl. In embodiments, R⁵ is substituted or unsubstituted group selected from methyl, cyclopropyl, cyclobutyl, cyclopentyl, or cyclohexyl, azetidine, pyrrolidine, piperidine, piperazine, morpholine, pyridine, thiazole, imidazole, pyrazole, or triazole.

In embodiments of the compounds of formula (1), (Ia), or (Ib), R^{F} is H or methyl. In embodiments of the compounds of formula (I), (Ia), or (Ib), R^{F} is H.

In embodiments of the compounds of formula (I), (Ia), or (Ib), one of X¹ and X² is N. In embodiments, X¹ is N and X² CH. In embodiments, X² is N and X¹ CH. In embodiments, X¹ and X² are both CH.

In embodiments, the compounds of the formula (I) have the structure of formula (II), or a pharmaceutically acceptable salt, tautomer, or stereoisomer thereof:

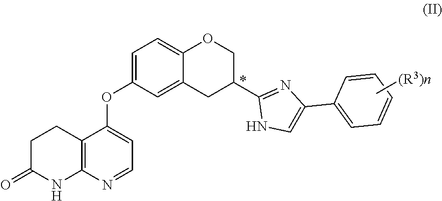

wherein, R³ is halogen, —OR^{A}, —NR^{A}R^{B}, —SO₂R^{C}, —SOR^{C}, —CN, C₁₋₄ alkyl, C₁₋₄ haloalkyl, or C₃₋₆ cycloalkyl, wherein the alkyl, haloalkyl and cycloalkyl groups are optionally substituted with 1 to 3 groups independently selected from: —OR^{A}, —CN, —SOR^{C}, or —NR^{A}R^{B};
wherein R^{A} and R^{B} are each independently selected from H, C₁₋₄ alkyl and C₁₋₄ haloalkyl;
wherein R^{C} is selected from C₁₋₄ alkyl and C₁₋₄ haloalkyl; and
n is 0, 1, 2, 3, or 4.

In embodiments, the compounds of the formula (II) has the following stereochemistry:

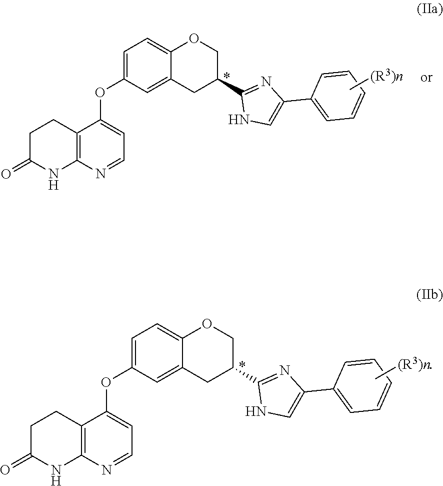

In embodiments, the compounds of the formula (II) has the stereochemistry as shown in formula (IIb).

In embodiments of the compounds of formula (II), (IIa), or (IIb), n is 0, 1, 2, or 3. In embodiments of the compounds of formula (II), (IIa), or (IIb), n is 0, 1, or 2. In embodiments of the compounds of formula (II), (IIa), or (IIb), n is 0, or 1. In embodiments of the compounds of formula (II), (IIa), or (IIb), nis 1.

In embodiments of the compounds of formula (II), (IIa), or (IIb), R³ is halogen, C₁₋₄ alkyl, —SO₂(C₁₋₄ alkyl). In embodiments of the compounds of formula (II), (IIa), or (IIb), R³ is halogen. In embodiments of the compounds of formula (II), (IIa), or (IIb), R³ is F.

In embodiments, the compounds of formula (I) or (II), or a pharmaceutically acceptable salt or tautomer thereof, have (S)-stereochemistry at the carbon marked with a *. In embodiments, the compounds of formula (I) or (II) having (S)-stereochemistry at the carbon marked with a * have greater than 80% enantiomeric excess (ee or e.e.), greater than 85% ee, greater than 90% ee, or greater than 95% ee. In embodiments, the compounds of formula (I) or (II) having (S)-stereochemistry at the carbon marked with a * have greater than 80% ee, 81% ee, 82% ee, 83% ee, 84% ee, 85% ee, 86% ee, 87% ee, 88% ee, 89% ee, 90% ee, 91% ee, 92% ee, 93% ee, 94% ee, or 95% ee, including all values therebetween.

In embodiments, the compounds of formula (I) or (II), or a pharmaceutically acceptable salt or tautomer thereof, have (R)-stereochemistry at the carbon marked with a *. In embodiments, the compounds of formula (I) or (II) having (R)-stereochemistry at the carbon marked with a * have greater than 80% enantiomeric excess (ee), greater than 85% ee. greater than 90% ee, or greater than 95% ee. In embodiments, the compounds of formula (1) or (II) having (R)-stereochemistry at the carbon marked with a * have greater than 80% ee, 81% ee, 82% ee, 83% ee, 84% ee, 85% ee, 86% ee, 87% ee, 88% ee, 89% ee, 90% ee, 91% ee, 92% ee, 93% ee, 94% ee, or 95% ee, including all values therebetween.

In embodiments, the compounds of formula (I), (Ia), (Ib), (II), (IIa), or (IIb), or a pharmaceutically acceptable salt thereof have a chemical purity of greater than 80%, 81%, 82%, 83%, 84%, 85%, 86%, 87%, 88%, 89%, 90%, 91%, 92%, 93%, 94%, 95%, 96%, 97%, 98%, or 99%, including all values therebetween.

In one embodiment, the compounds of formula (I), (Ia), or (Ib) is selected from Table A, or a pharmaceutically acceptable salt or tautomer thereof. In one embodiment, the compound of formula (Ia) or (Ib) is selected from Compound A-1, A-2, B-1, or B-2, or a pharmaceutically acceptable salt or tautomer thereof.

TABLE A

| Compound ID | Structure |
|---|---|
| A-rac | 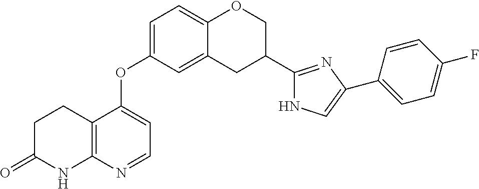 [SMILES: O=C1CCc2c(Oc3ccc4c(c3)CC(c3nc(-c5ccc(F)cc5)c[nH]3)CO4)ccnc2N1] |
| A-1 (S) isomer (faster eluting isomer by chiral HPLC method as described in Example 3) | 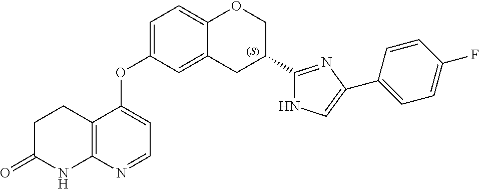 |
| A-2 (R) isomer (slower eluting isomer by chiral HPLC method as described in Example 3) | 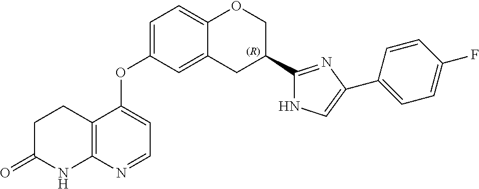 |
| B-rac | 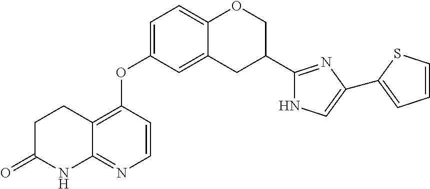 [SMILES: O=C1CCc2c(Oc3ccc4c(c3)CC(c3nc(-c5cccs5)c[nH]3)CO4)ccnc2N1] |
| B-1 (S) isomer | 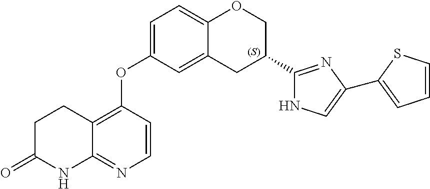 |
| B-2 (R) isomer | 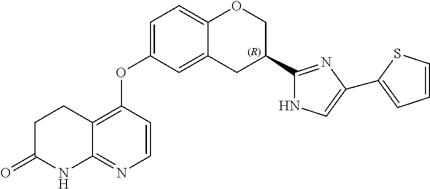 |
| C-rac | 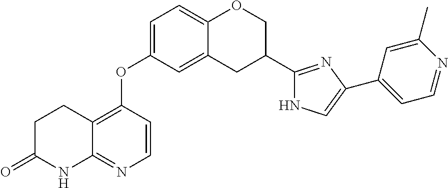 [SMILES: Cc1cc(-c2c[nH]c(C3COc4ccc(Oc5ccnc6c5CCC(=O)N6)cc4C3)n2)ccn1] |
| C-1 | 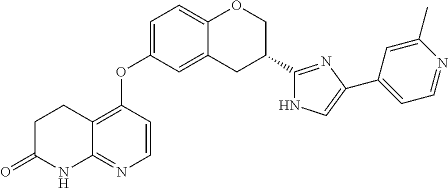 [SMILES: Cc1cc(-c2c[nH]c([C@H]3COc4ccc(Oc5ccnc6c5CCC(=O)N6)cc4C3)n2)ccn1] |
| C-2 | 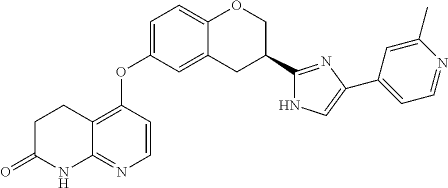 [SMILES: Cc1cc(-c2c[nH]c([C@@H]3COc4ccc(Oc5ccnc6c5CCC(=O)N6)cc4C3)n2)ccn1] |
| D-rac | 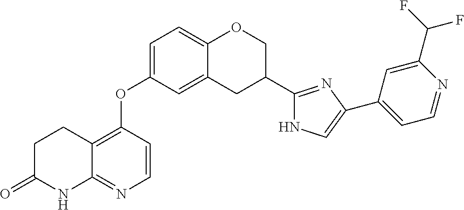 [SMILES: O=C1CCc2c(Oc3ccc4c(c3)CC(c3nc(-c5ccnc(C(F)F)c5)c[nH]3)CO4)ccnc2N1] |
| D-1 | 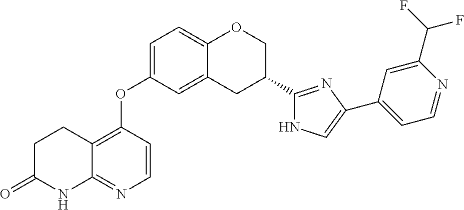 [SMILES: O=C1CCc2c(Oc3ccc4c(c3)C[C@@H](c3nc(-c5ccnc(C(F)F)c5)c[nH]3)CO4)ccnc2N1] |
| D-2 | 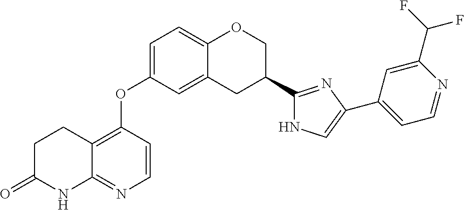 [SMILES: O=C1CCc2c(Oc3ccc4c(c3)C[C@H](c3nc(-c5ccnc(C(F)F)c5)c[nH]3)CO4)ccnc2N1] |
| E-rac | 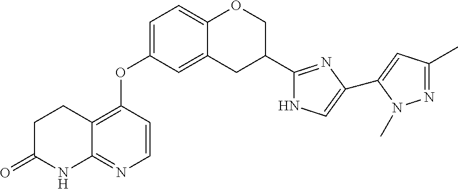 [SMILES: Cc1cc(-c2c[nH]c(C3COc4ccc(Oc5ccnc6c5CCC(=O)N6)cc4C3)n2)n(C)n1] |
| E-1 | 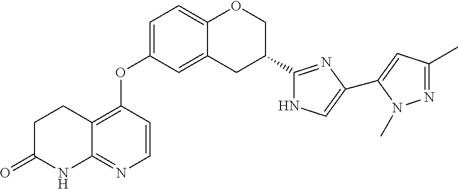 [SMILES: Cc1cc(-c2c[nH]c([C@H]3COc4ccc(Oc5ccnc6c5CCC(=O)N6)cc4C3)n2)n(C)n1] |
| E-2 | 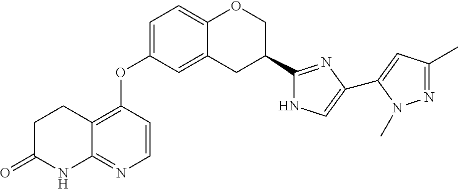 [SMILES: Cc1cc(-c2c[nH]c([C@@H]3COc4ccc(Oc5ccnc6c5CCC(=O)N6)cc4C3)n2)n(C)n1] |
| F-rac | 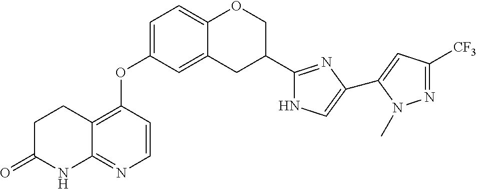 [SMILES: Cn1nc(C(F)(F)F)cc1-c1c[nH]c(C2COc3ccc(Oc4ccnc5c4CCC(=O)N5)cc3C2)n1] |
| F-1 | 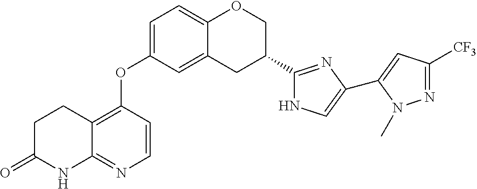 [SMILES: Cn1nc(C(F)(F)F)cc1-c1c[nH]c([C@H]2COc3ccc(Oc4ccnc5c4CCC(=O)N5)cc3C2)n1] |
| F-2 | 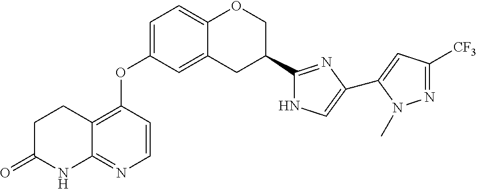 [SMILES: Cn1nc(C(F)(F)F)cc1-c1c[nH]c([C@@H]2COc3ccc(Oc4ccnc5c4CCC(=O)N5)cc3C2)n1] |
| G-rac | 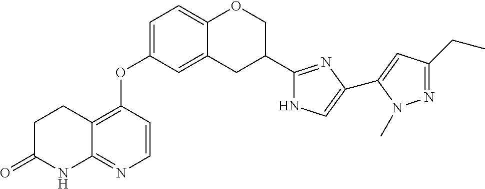 [SMILES: CCc1cc(-c2c[nH]c(C3COc4ccc(Oc5ccnc6c5CCC(=O)N6)cc4C3)n2)n(C)n1] |
| G-1 | 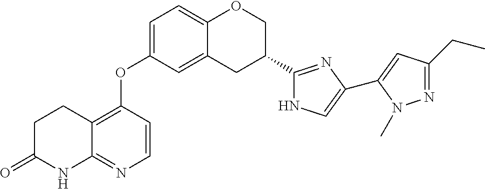 [SMILES: CCc1cc(-c2c[nH]c([C@H]3COc4ccc(Oc5ccnc6c5CCC(=O)N6)cc4C3)n2)n(C)n1] |
| G-2 | 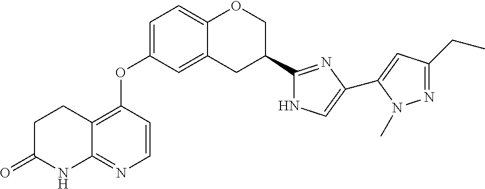 [SMILES: CCc1cc(-c2c[nH]c([C@@H]3COc4ccc(Oc5ccnc6c5CCC(=O)N6)cc4C3)n2)n(C)n1] |
| H-rac | 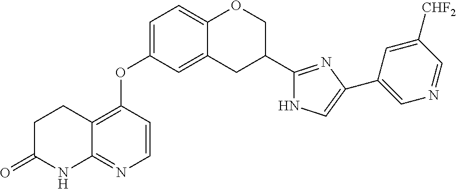 [SMILES: O=C1CCc2c(Oc3ccc4c(c3)CC(c3nc(-c5cncc(C(F)F)c5)c[nH]3)CO4)ccnc2N1] |
| H-1 | 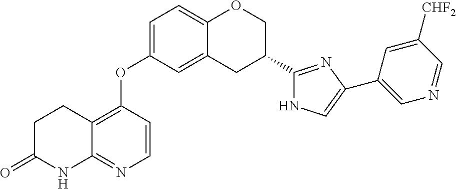 [SMILES: O=C1CCc2c(Oc3ccc4c(c3)C[C@@H](c3nc(-c5cncc(C(F)F)c5)c[nH]3)CO4)ccnc2N1] |
| H-2 | 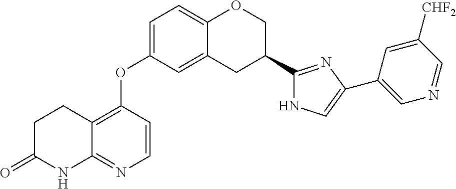 [SMILES: O=C1CCc2c(Oc3ccc4c(c3)C[C@H](c3nc(-c5cncc(C(F)F)c5)c[nH]3)CO4)ccnc2N1] |
| J-rac | 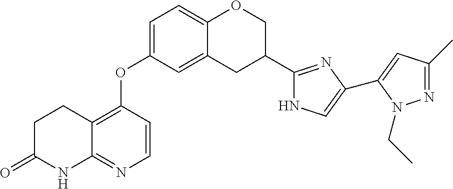 [SMILES: CCn1nc(C)cc1-c1c[nH]c(C2COc3ccc(Oc4ccnc5c4CCC(=O)N5)cc3C2)n1] |
| J-1 | 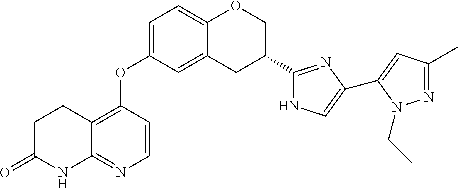 [SMILES: CCn1nc(C)cc1-c1c[nH]c([C@H]2COc3ccc(Oc4ccnc5c4CCC(=O)N5)cc3C2)n1] |
| J-2 | 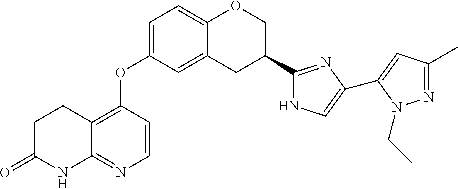 [SMILES: CCn1nc(C)cc1-c1c[nH]c([C@@H]2COc3ccc(Oc4ccnc5c4CCC(=O)N5)cc3C2)n1] |
| K-rac | 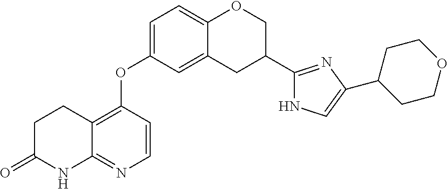 [SMILES: O=C1CCc2c(Oc3ccc4c(c3)CC(c3nc(C5CCOCC5)c[nH]3)CO4)ccnc2N1] |
| K-1 | 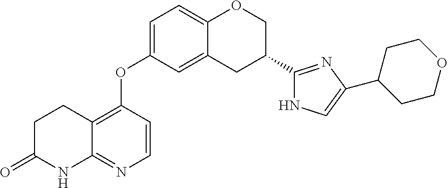 [SMILES: O=C1CCc2c(Oc3ccc4c(c3)C[C@@H](c3nc(C5CCOCC5)c[nH]3)CO4)ccnc2N1] |
| K-2 | 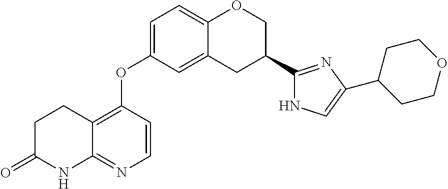 [SMILES: O=C1CCc2c(Oc3ccc4c(c3)C[C@H](c3nc(C5CCOCC5)c[nH]3)CO4)ccnc2N1] |
| L-rac | 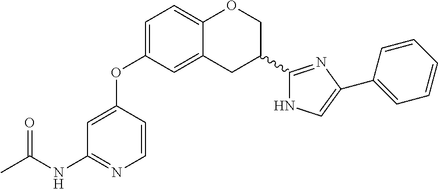 [SMILES: CC(=O)Nc1cc(Oc2ccc3c(c2)CC(c2nc(-c4ccccc4)c[nH]2)CO3)ccn1] |
| L-1 | 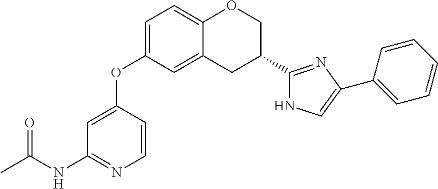 [SMILES: CC(=O)Nc1cc(Oc2ccc3c(c2)C[C@@H](c2nc(-c4ccccc4)c[nH]2)CO3)ccn1] |
| L-2 | 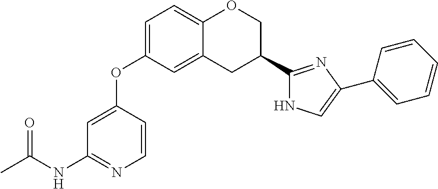 [SMILES: CC(=O)Nc1cc(Oc2ccc3c(c2)C[C@H](c2nc(-c4ccccc4)c[nH]2)CO3)ccn1] |
| M-rac | 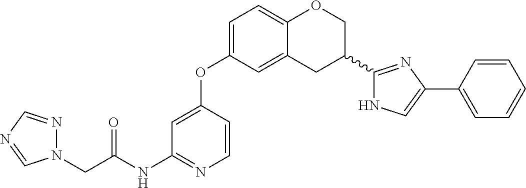 [SMILES: O=C(Cn1cncn1)Nc1cc(Oc2ccc3c(c2)CC(c2nc(-c4ccccc4)c[nH]2)CO3)ccn1] |
| M-1 | 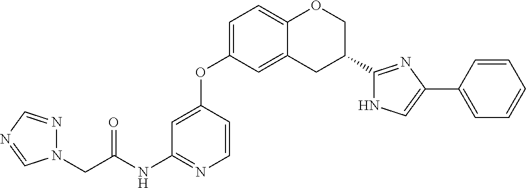 [SMILES: O=C(Cn1cncn1)Nc1cc(Oc2ccc3c(c2)C[C@@H](c2nc(-c4ccccc4)c[nH]2)CO3)ccn1] |
| M-2 | 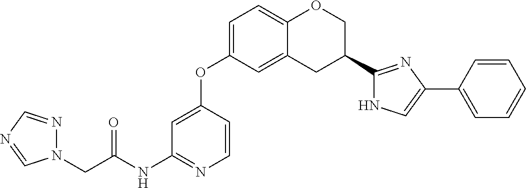 [SMILES: O=C(Cn1cncn1)Nc1cc(Oc2ccc3c(c2)C[C@H](c2nc(-c4ccccc4)c[nH]2)CO3)ccn1] |
| N-rac | 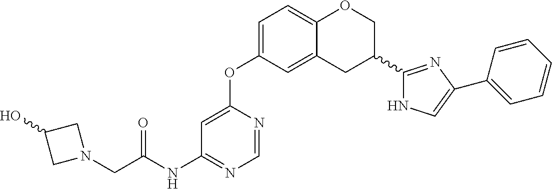 [SMILES: O=C(CN1CC(O)C1)Nc1cc(Oc2ccc3c(c2)CC(c2nc(-c4ccccc4)c[nH]2)CO3)ncn1] |
| N-1 | 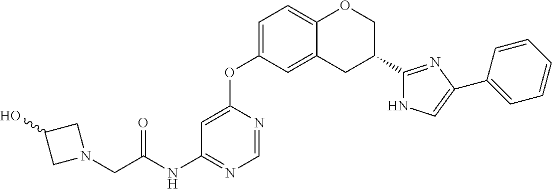 [SMILES: O=C(CN1CC(O)C1)Nc1cc(Oc2ccc3c(c2)C[C@@H](c2nc(-c4ccccc4)c[nH]2)CO3)ncn1] |
| N-2 | 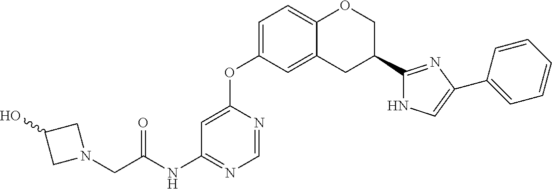 [SMILES: O=C(CN1CC(O)C1)Nc1cc(Oc2ccc3c(c2)C[C@H](c2nc(-c4ccccc4)c[nH]2)CO3)ncn1] |

### Chiral Synthesis of the Compounds of the Invention

The present disclosure relates chiral synthesis of Compounds of formula (I), (Ia), (Ib), (II), (IIa) or (IIb), or a pharmaceutically acceptable salt, tautomer, or stereoisomer thereof.

In embodiments, the chiral synthesis uses (S)-6-hydroxychromane-3-carboxylic acid or (R)-6-hydroxychromane-3-carboxylic acid. In embodiments, (S)-6-hydroxychromane-3-carboxylic acid or (R)-6-hydroxychromane-3-carboxylic acid used in the chiral synthesis has an enantiomeric excess of at least 85%, at least 90%, or at least 95%. In embodiments, (S)-6-hydroxychromane-3-carboxylic acid or (R)-6-hydroxychromane-3-carboxylic acid used in the chiral synthesis has an enantiomeric excess of about 80% ee, 81% ee, 82% ee, 83% ee, 84% ee, 85% ee, 86% ee, 87% ee, 88% ee, 89% ee, 90% ee, 91% ee, 92% ee, 93% ee, 94% ee, or 95% ee, including all values therebetween.

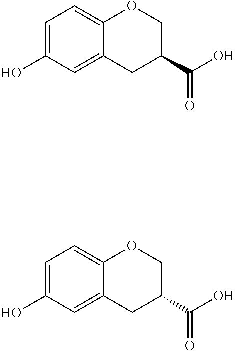 [SMILES: O=C(O)[C@@H]1COc2ccc(O)cc2C1 ; O=C(O)[C@H]1COc2ccc(O)cc2C1]

In embodiments, (S)-6-hydroxychromane-3-carboxylic acid or (R)-6-hydroxychromane-3-carboxylic acid is prepared from 6-hydroxy-2H-chromene-3-carboxylic acid by chiral hydrogenation as shown in Scheme 1. In embodiments, the chiral hydrogenation uses a transition metal catalyst. In embodiments, the chiral hydrogenation uses a Ru or Rₕ catalyst. In embodiments, the chiral hydrogenation uses a Ru catalyst selected from Ru(OAc)₂, [RuCl₂(p-cym)]₂, Ru(COD)(Me-allyl)₂, or Ru(COD)(TFA)₂. In embodiments, Ru catalyst selected from [RuCl₂(p-cym)]₂, Ru(COD)(Me-allyl)₂, or Ru(COD)(TFA)₂. In n embodiments, the chiral hydrogenation uses a Rₕ catalyst selected from [Rh(COD)₂]OTf or [Rh(COD)₂]BF₄.

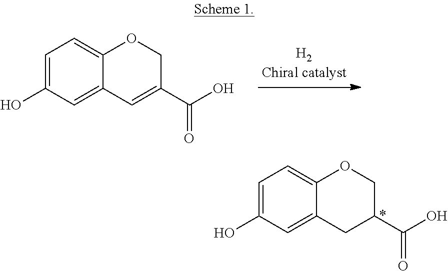 [SMILES: O=C(O)C1=Cc2cc(O)ccc2OC1 ; O=C(O)C1COc2ccc(O)cc2C1]

In embodiments, the chiral hydrogenation uses a chiral ligand. In embodiments, the chiral phosphine ligands. In embodiments, the chiral ligand is selected from Table B, or an opposite chiral ligand thereof (i.e., where Table B list (S)-PhanePhos, the disclosure expressly includes the opposite chiral ligand (R)-PhanePhos). In embodiments, the chiral ligand is selected from Table 4A or Table 5, or an opposite chiral ligand thereof.

In embodiments, the chiral hydrogenation of Scheme 1 uses (R)-PhanePhos in combination with a catalyst. In embodiments, the chiral hydrogenation of Scheme 1 uses (R)-PhanePhos in combination with a Ru catalyst. In embodiments, the chiral hydrogenation of Scheme 1 uses (R)-PhanePhos with [RuCl₂(p-cym)]₂.

TABLE B
Chiral Ligands

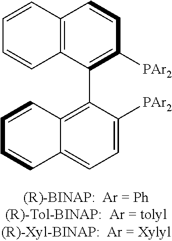

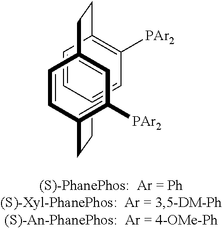

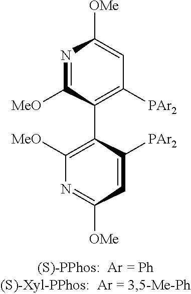

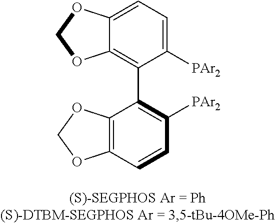

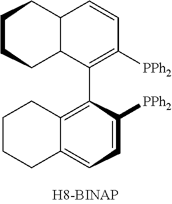

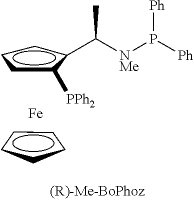

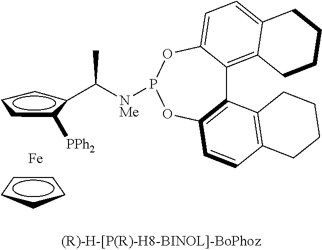

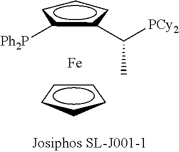

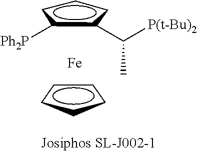

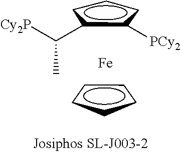

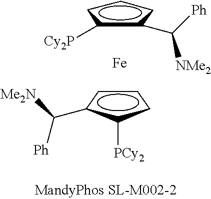

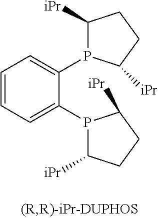 [SMILES: CC(C)[C@H]1CC[C@H](C(C)C)P1c1ccccc1P1[C@@H](C(C)C)CC[C@@H]1C(C)C]

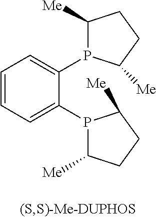 [SMILES: C[C@H]1CC[C@H](C)P1c1ccccc1P1[C@@H](C)CC[C@@H]1C]

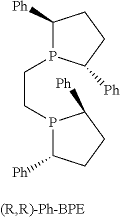 [SMILES: c1ccc([C@H]2CC[C@H](c3ccccc3)P2CCP2[C@@H](c3ccccc3)CC[C@@H]2c2ccccc2)cc1]

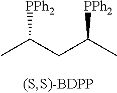

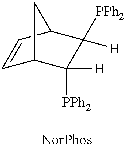

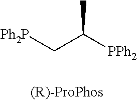

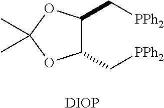

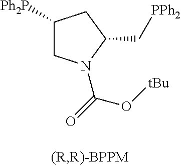

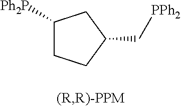

In embodiments of the chiral hydrogenation, the chiral ligand is selected from (S)- or (R)-BINAP, (S)- or (R)-H8-BINAP, (S)- or (R)-PPhos, (S)- or (R)-Xyl-PPhos, (S)- or (R)-PhanePhos, (S)- or (R)-Xyl-PhanePhos, (S,S)-Me-DuPhos, (R,R)-Me-DuPhos, (S,S)-iPr-DuPhos, (R,R)-iPr-DuPhos, (S,S)-NorPhos, (R,R)-NorPhos, (S,S)-BPPM, or (R,R)-BPPM, Josiphos SL-J002-1. In embodiments, the chiral ligand is (S)- or (R)-PhanePhos or (S)- or (R)-An-PhanePhos. In embodiments, the chiral ligand is (S)- or (R)-PhanePhos. In embodiments, the chiral ligand is (R)-PhanePhos.

In embodiments of the chiral hydrogenation, metal catalyst precursor and chiral ligand are used to form a chiral metal complex in situ. In embodiments, the metal catalyst precursor is selected from any one of Rh or Ru catalyst disclosed herein, and the chiral ligand is selected from any one of the chiral ligands disclosed herein. In embodiments, the metal catalyst precursor is Ru(OAc)₂, [RuCl₂(p-cym)]₂, Ru(COD)(Me-allyl)₂, or Ru(COD)(TFA)₂ and the chiral ligand is (S) or (R)-PhanePhos or (S)- or (R)-An-PhanePhos. In embodiments, the metal catalyst precursor is [RuCl₂(p-cym)]₂, Ru(COD)(Me-allyl)₂, or Ru(COD)(TFA)₂ and the chiral ligand is (S)- or (R)-PhanePhos. In embodiments, the metal catalyst precursor and the chiral ligand are used at a ratio in the range of about 1:2 to about 1:1, including all values and ranges therebetween. In embodiments, the metal catalyst precursor and the chiral ligand are used at a ratio in the range of about 1:1 to about 1:1.5, including all values and ranges therebetween. In embodiments, the metal catalyst precursor and the chiral ligand are used at a ratio of about 1:1, about 1:1.1, about 1:1.2, about 1:1.3, about 1:1.4, or about 1:1.5.

In embodiments, the metal catalyst precursor is [RuCl₂(p-cym)]₂ and the chiral ligand is (R)-PhanePhos. In embodiments, the metal catalyst precursor and the chiral ligand are used at a ratio in the range of about 1:2 to about 1:1, including all values and ranges therebetween. In embodiments, the metal catalyst precursor and the chiral ligand are used at a ratio of about 1:2.

In embodiments, the metal catalyst precursor and the chiral ligand is pre-mixed to pre-form the chiral metal complex prior to setting up the hydrogenation reaction. In embodiments, the pre-formed chiral metal complex is selected from [(R)-Phanephos-RuCl₂(p-cym)], [(S)-Phanephos-RuCl₂(p-cym)], [(R)-An-Phanephos-RuCl₂(p-cym)], [(S)-An-Phanephos-RuCl₂(p-cym)], [(R)-BINAP-RuCl(p-cym)]Cl, [(S)-BINAP-RuCl(p-cym)]Cl, (R)-BINAP-Ru(OAc)₂, (S)-BINAP-Ru(OAc)₂, [(R)-Phanephos-Rh(COD)]BF₄, [(S)-Phanephos-Rh(COD)]BF₄, [(R)-Phanephos-Rh(COD)]OTf, or [(S)-Phanephos-Rh(COD)]OTf. In embodiments, the pre-formed chiral metal complex is [(R)-Phanephos-RuCl₂(p-cym)], [(S)-Phanephos-RuCl₂(p-cym)], [(R)-An-Phanephos-RuCl₂(p-cym)], or [(S)-An-Phanephos-RuCl₂(p-cym)]. In embodiments, the pre-formed chiral metal complex is [(R)-Phanephos-RuCl₂(p-cym)] or [(S)-Phanephos-RuCl₂(p-cym)].

In embodiments, the metal catalyst precursor and the chiral ligand does not require to be pre-mixed to pre-form the chiral metal complex prior to setting up the hydrogenation reaction.

In embodiments of the chiral hydrogenation, a catalyst loading in the range of about 20/1 (substrate/catalyst=S/C) to about 2,000/1, including all values and ranges therebetween is used. In embodiments, the catalyst loading (S/C) is in the range of about 25/1 to about 1,000/1, including all values and ranges therebetween. In embodiments, the catalyst loading (S/C) is in the range of about 200/1 to about 1,000/1, including all values and ranges therebetween. In embodiments, the catalyst loading (S/C) is about 25/1, about 50/1, about 100/1, about 150/1, about 200/1, about 250/1, about 300/1, about 350/1, about 400/1, about 450/1, about 500/1, about 550/1, about 600/1, about 650/1, about 700/1, about 750/1, about 800/1, about 850/1, about 900/1, about 950/1, about 1,000/1, about 1,100/1, about 1,200/1, about 1,300/1, about 1,400/1, about 1,500/1, about 1,600/1, about 1,700/1, about 1,800/1, about 1,900/1, or about 2,000/1, including all values therebetween. In embodiments, the catalyst loading (S/C) is in the range of about 200/1 to about 500/1, including all values and ranges therebetween. In embodiments, the catalyst loading (S/C) is in the range of about 300/1 to about 350/1, including all values and ranges therebetween. In embodiments, the catalyst loading (S/C) is in the range of about 320/1 to about 330/1, including all values and ranges therebetween.

In embodiments of the chiral hydrogenation, a base is used. In embodiments, the base is selected from amines. In embodiments, the base is selected from triethylamine, NaOMe or Na₂CO₃. In embodiments, the base is triethylamine. In embodiments, the base is used in ≤2 equivalent with respect to 6-hydroxy-2H-chromene-3-carboxylic acid. In embodiments, the base is used in ≤2 equivalent with respect to 6-hydroxy-2H-chromene-3-carboxylic acid. In embodiments, the base is used in about 1.5 equivalent with respect to 6-hydroxy-2H-chromene-3-carboxylic acid.

In embodiments of the chiral hydrogenation, the base is used in substoichiometric amounts with respect to 6-hydroxy-2H-chromene-3-carboxylic acid. In one embodiment, the base is used in about 0.9, 0.8, 0.7, 0.6, 0.5, 0.4, 0.3, 0.2, or 0.1 equivalent with respect to 6-hydroxy-2H-chromene-3-carboxylic acid, including all values therebetween. In one embodiment, the base is used in about 0.1 equivalent with respect to 6-hydroxy-2H-chromene-3-carboxylic acid.

In embodiments of the chiral hydrogenation, the reaction is performed at a temperature in the range of about 25° C. to about 70° C., including all values and ranges therebetween. In embodiments, the chiral hydrogenation, the reaction is performed at a temperature in the range of about 25° C. to about 70° C., including all values and ranges therebetween. In embodiments, the chiral hydrogenation, the reaction is performed at a temperature in the range of about 30° C. to about 40° C., including all values and ranges therebetween. In embodiments, the chiral hydrogenation, the reaction is performed at about 30° C. to about 40° C. In embodiments, the chiral hydrogenation, the reaction is performed at about 40° C.

In embodiments of the chiral hydrogenation, the substrate concentration ([S], i.e., concentration of 6-hydroxy-2H-chromene-3-carboxylic acid) is in the range of about 0.01M to about 5M, including all values and ranges therebetween. In embodiments, [S] is in the range of about 0.1M to about 1M, including all values and ranges therebetween. In embodiments, [S] is in the range of about 0.2M to about 0.8M, including all values and ranges therebetween. In embodiments, [S] is about 0.2M, 0.3M, 0.4M, 0.5M, 0.6M, 0.7M, or 0.8M, including all values therebetween. In embodiments, [S] is about 0.5M.

In embodiments of the chiral hydrogenation, the pressure for H₂ is in the range of about 1 bar to about 50 bar, including all values and ranges therebetween. In embodiments, the pressure for H2 is in the range of about 2 bar to about 30 bar, including all values and ranges therebetween. In embodiments, the pressure for H2 is in the range of about 3 bar to about 10 bar, including all values and ranges therebetween. In embodiments, the pressure for H2 is in the range of about 5 bar to about 6 bar. In embodiments, the pressure for H2 is about 5 bar.

In embodiments of the chiral hydrogenation, the solvent is a protic solvent. In embodiments of the chiral hydrogenation, the solvent is an alcohol solvent. In embodiments of the chiral hydrogenation, the solvent is methanol, ethanol, isopropanol, or fluorinated variants thereof (such as trifluoroethanol). In embodiments of the chiral hydrogenation, the solvent is methanol. In embodiments of the chiral hydrogenation, the solvent is ethanol.

In embodiments of the chiral hydrogenation, to achieve a high % ee of (S)-6-hydroxychromane-3-carboxylic acid or (R)-6-hydroxychromane-3-carboxylic acid, an inert vessel free of contaminants is desired. In embodiments, to achieve a high % ee of the products, the vessel should be free of metal deposit contaminants.

In embodiments of the chiral hydrogenation of Scheme 1, the chiral purity of (S)-6-hydroxychromane-3-carboxylic acid or (R)-6-hydroxychromane-3-carboxylic acid is greater than about 90%. In embodiments, the chiral purity of (S)-6-hydroxychromane-3-carboxylic acid or (R)-6-hydroxychromane-3-carboxylic acid is greater than about 90%, about 91%, about 92%, about 93%, about 94%, about 95%, or about 96%. In embodiments, the chiral purity of (S)-6-hydroxychromane-3-carboxylic acid or (R)-6-hydroxychromane-3-carboxylic acid is greater than about 95%.

In embodiments, the chiral synthesis of Compounds of formula (I), (Ia), (Ib), (II), (IIa) or (IIb), or a pharmaceutically acceptable salt, tautomer, or stereoisomer thereof, comprises a reaction step labeled as Scheme 2A, wherein X¹, X², R⁶, and R⁷ are as described herein.

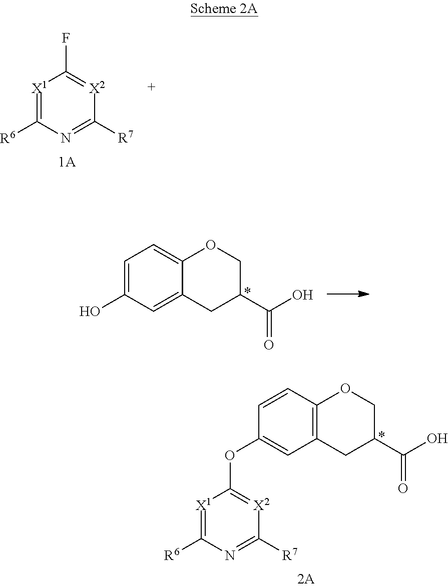

In embodiments, the chiral synthesis of Compounds of formula (I), (Ia), (Ib), (II), (IIa) or (IIb), or a pharmaceutically acceptable salt, tautomer, or stereoisomer thereof, comprises a reaction step labeled as Scheme 2B.

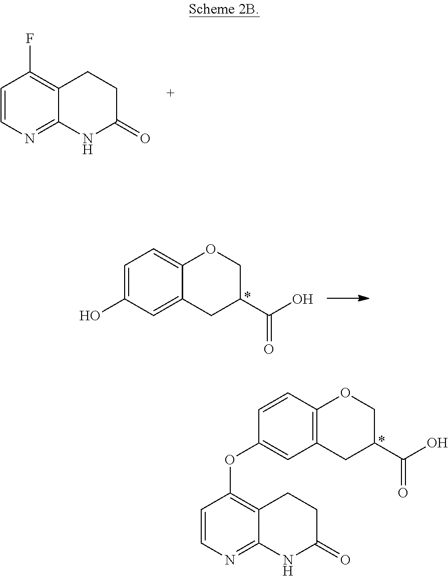 [SMILES: O=C1CCc2c(F)ccnc2N1 ; O=C(O)C1COc2ccc(O)cc2C1 ; O=C1CCc2c(Oc3ccc4c(c3)CC(C(=O)O)CO4)ccnc2N1]

In embodiments of Scheme 2A or 2B, (S)-6-hydroxychromane-3-carboxylic acid or (R)-6-hydroxychromane-3-carboxylic acid has an enantiomeric excess of at least 85%, at least 90%, at least 95%, or at least 98%.

In embodiments of Scheme 2A or 2B, when (R)-6-hydroxychromane-3-carboxylic acid is used, the stereochemistry of (R)-6-hydroxychromane-3-carboxylic acid is retained in the product (e.g., (3R)-6-[(7-oxo-5,6,7,8-tetrahydro-1,8-naphthyridin-4-yl)oxy]-3,4-dihydro-2H-1-benzopyran-3-carboxylic acid). In embodiments of Scheme 2A or 2B, when (S)-6-hydroxychromane-3-carboxylic acid is used, the stereochemistry of (S)-6-hydroxychromane-3-carboxylic acid is retained in the product (e.g., (3S)-6-[(7-oxo-5,6,7,8-tetrahydro-1,8-naphthyridin-4-yl)oxy]-3,4-dihydro-2H-1-benzopyran-3-carboxylic acid).

In embodiments of Scheme 2A or 2B, using (R)-6-hydroxychromane-3-carboxylic acid provides the product as an (R) isomer. In embodiments of Scheme 2B, using (R)-6-hydroxychromane-3-carboxylic acid provides (3R)-6-[(7-oxo-5,6,7,8-tetrahydro-1,8-naphthyridin-4-yl)oxy]-3,4-dihydro-2H-1-benzopyran-3-carboxylic acid. In embodiments, the chiral purity of (3R)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is within 10% of the chiral purity of (R)-6-hydroxychromane-3-carboxylic acid used in the reaction. In embodiments, the chiral purity of (3R)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is within 5% of the chiral purity of (R)-6-hydroxychromane-3-carboxylic acid used in the reaction. In embodiments, the chiral purity of (3R)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is greater than 90% when prepared from (R)-6-hydroxychromane-3-carboxylic acid having a chiral purity of greater than 90%. In embodiments, the chiral purity of (3R)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is greater than 95% when prepared from (R)-6-hydroxychromane-3-carboxylic acid having a chiral purity of greater than 95%. In embodiments, the chiral purity of (3R)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is greater than about 98% when prepared from (R)-6-hydroxychromane-3-carboxylic acid having a chiral purity of greater than about 98%.

In embodiments of Scheme 2A or 2B, using (S)-6-hydroxychromane-3-carboxylic acid provides the product as an (S) isomer. In embodiments of Scheme 2B, using (S)-6-hydroxychromane-3-carboxylic acid provides (3S)-6-[(7-oxo-5,6,7,8-tetrahydro-1,8-naphthyridin-4-yl)oxy]-3,4-dihydro-2H-1-benzopyran-3-carboxylic acid. In embodiments, the chiral purity of (3S)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is within 10% of the chiral purity of (S)-6-hydroxychromane-3-carboxylic acid used in the reaction. In embodiments, the chiral purity of (3S)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is within 5% of the chiral purity of (S)-6-hydroxychromane-3-carboxylic acid used in the reaction. In embodiments, the chiral purity of (3S)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is greater than 90% when prepared from (S)-6-hydroxychromane-3-carboxylic acid having a chiral purity of greater than 90%. In embodiments, the chiral purity of (3S)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is greater than 95% when prepared from (S)-6-hydroxychromane-3-carboxylic acid having a chiral purity of greater than 95%. In embodiments, the chiral purity of (3S)-6-[(7-oxo-5,6,7,8-tetrahydro-1,8-naphthyridin-4-yl)oxy]-3,4-dihydro-2H-1-benzopyran-3-carboxylic acid prepared by Scheme B reaction is greater than about 98% when prepared from (S)-6-hydroxychromane-3-carboxylic acid having a chiral purity of greater than about 98%.

In embodiments of Scheme 2A or 2B, a base is used. In embodiments, the base is potassium carbonate. In embodiments, the base is tribasic potassium phosphate (K₃PO₄).

In embodiments of Scheme 2A or 2B, reaction is heated to a temperature in the range of about 30° C. to about 150° C., including all values and ranges therebetween. In embodiments, the reaction of Scheme 2A or 2B is heated to a temperature in the range of about 75° C. to about 150° C., including all values and ranges therebetween. In embodiments, the reaction of Scheme 2A or 2B is heated to a temperature in the range of about 80° C. to about 120° C., including all values and ranges therebetween. In embodiments, the reaction of Scheme 2A or 2B is heated to a temperature in the range of about 90° C. to about 110° C., including all values and ranges therebetween.

In embodiments, the chiral synthesis of Compounds of formula (I), (Ia) or (Ib), or a pharmaceutically acceptable salt, tautomer, or stereoisomer thereof, comprises a reaction step labeled as Scheme 3A.

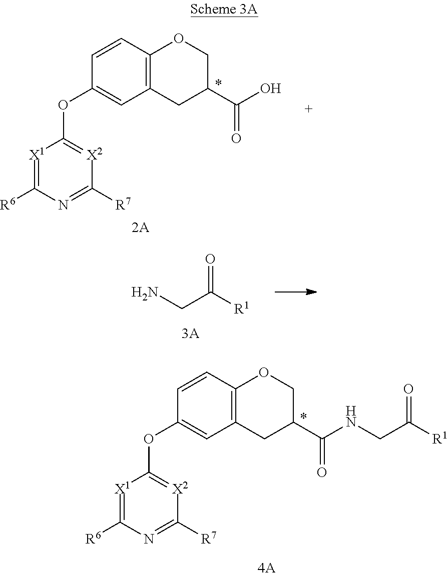

In embodiments of Scheme 3A, the compound of formula 2A has a (R) or (S) stereochemistry at the position labeled with *. In embodiments of Scheme 3A, the compound of formula 2A has an enantiomeric excess of at least 85%, at least 90%, at least 95%, or at least 98%.

In embodiments, the chiral synthesis of Compounds of formula (I), (Ia) or (Ib), or a pharmaceutically acceptable salt, tautomer, or stereoisomer thereof, comprises a reaction step labeled as Scheme 3B.

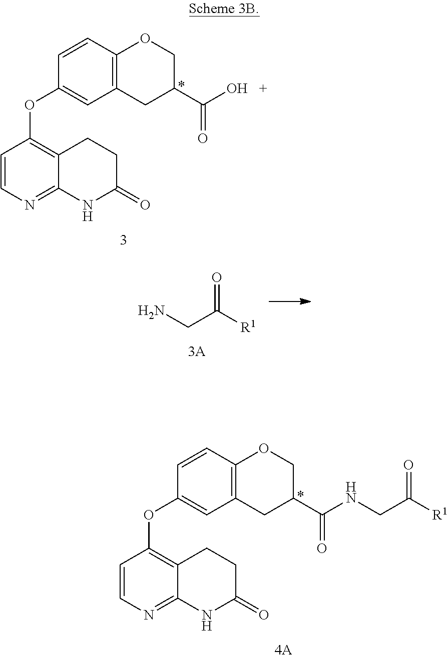

In embodiments, the chiral synthesis of Compounds of formula (II), (IIa) or (IIb), or a pharmaceutically acceptable salt, tautomer, or stereoisomer thereof, comprises a reaction step labeled as Scheme 3C.

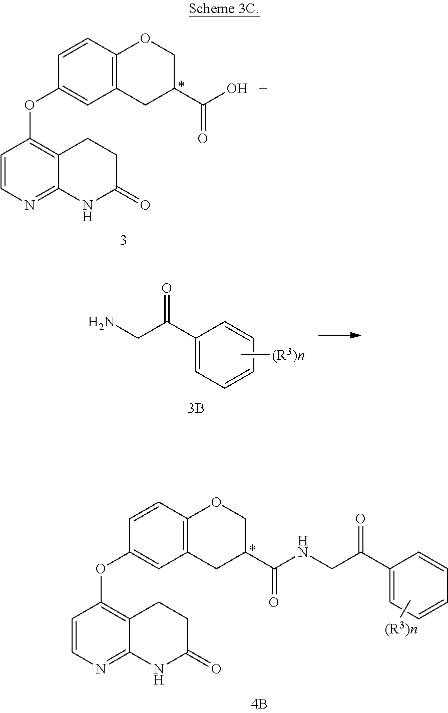

In embodiments of Scheme 3B or Scheme 3C, Compound 3 has a (R) or (S) stereochemistry at the position labeled with *. In embodiments of Scheme 3A or Scheme 3B, Compound 3 has an enantiomeric excess of at least 85%, at least 90%, at least 95%, or at least 98%.

In embodiments of Scheme 3A, Scheme 3B, or Scheme 3C, the reaction is performed in the presence of propylphosphonic anhydride (T3P) and N,N-diisopropylethylamine. In embodiments of Scheme 3A or Scheme 3B, Compound 3A can be in a form of a salt, such as hydrochloride salt. In embodiments of Scheme 3C, Compound 3B can be in a form of a salt, such as hydrochloride salt.

In embodiments of Scheme 3C, Compound 3B is 2-(4-fluorophenyl)-2-oxoethan-1-aminium chloride.

In embodiments, the chiral synthesis of Compounds of formula (I), (Ia) or (Ib), or a pharmaceutically acceptable salt, tautomer, or stereoisomer thereof, comprises a reaction step labeled as Scheme 4A.

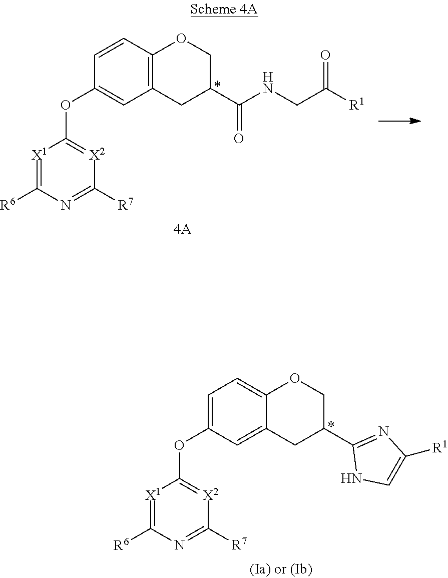

In embodiments, the chiral synthesis of Compounds of formula (I), (Ia) or (Ib), or a pharmaceutically acceptable salt, tautomer, or stereoisomer thereof, comprises a reaction step labeled as Scheme 4B.

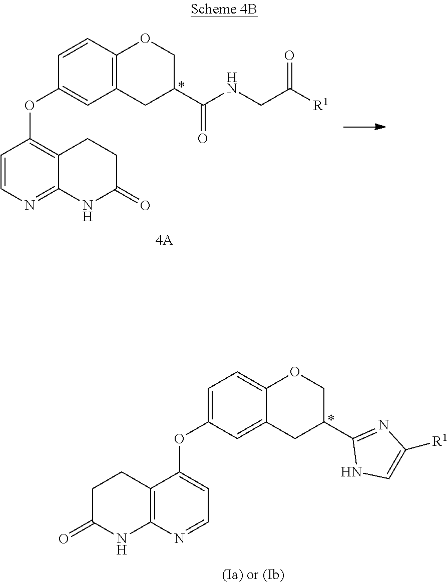

In embodiments of Scheme 4A or 4B, a Compound of formula 4A has a (R) or (S) stereochemistry at the position labeled with *. In embodiments of Scheme 4A or 4B, a Compound of formula 4A has an enantiomeric excess of at least 85%, at least 90%, at least 95%, or at least 98%.

In embodiments of Scheme 4A or 4B, when the stereochemistry of Compound 4A is retained in the product. In embodiments of Scheme 4A or 4B, when (S) enantiomer of Compound 4A is used, Compound of formula (Ia) is obtained. In embodiments of Scheme 4A or 4B, when (R) enantiomer of Compound 4A is used, Compound of formula (Ib) is obtained.

In embodiments, the chiral purity of a Compound of formula (Ia) prepared by Scheme 4A or 4B reaction is within 10% of the chiral purity of an (S) enantiomer of Compound 4A used in the reaction. In embodiments, the chiral purity of a Compound of formula (Ia) prepared by Scheme 4A or 4B reaction is within 5% of the chiral purity of an (S) enantiomer of Compound 4A used in the reaction. In embodiments, the chiral purity of a Compound of formula (Ia) prepared by Scheme 4A or 4B reaction is greater than 90% when prepared from an (S) enantiomer of Compound 4A having a chiral purity of greater than 90%. In embodiments, the chiral purity of a Compound of formula (Ia) prepared by Scheme 4A or 4B reaction is greater than 95% when prepared from an (S) enantiomer of Compound 4A having a chiral purity of greater than 95%. In embodiments, the chiral purity of a Compound of formula (Ia) prepared by Scheme 4A or 4B reaction is greater than 98% when prepared from an (S) enantiomer of Compound 4A having a chiral purity of greater than 98%.

In embodiments, the chiral purity of a Compound of formula (Ib) prepared by Scheme 4A or 4B reaction is within 10% of the chiral purity of an (R) enantiomer of Compound 4A used in the reaction. In embodiments, the chiral purity of a Compound of formula (Ib) prepared by Scheme 4A or 4B reaction is within 5% of the chiral purity of an (R) enantiomer of Compound 4A used in the reaction. In embodiments, the chiral purity of a Compound of formula (Ib) prepared by Scheme 4A or 4B reaction is greater than 90% when prepared from an (R) enantiomer of Compound 4A having a chiral purity of greater than 90%. In embodiments, the chiral purity of a Compound of formula (Ib) prepared by Scheme 4A or 4B reaction is greater than 95% when prepared from an (R) enantiomer of Compound 4A having a chiral purity of greater than 95%. In embodiments, the chiral purity of a Compound of formula (Ib) prepared by Scheme 4A or 4B reaction is greater than 98% when prepared from an (R) enantiomer of Compound 4A having a chiral purity of greater than 98%.

In embodiments of Scheme 4A or 4B, the reaction is performed in the presence of ammonia or an ammonium salt. In embodiments, the ammonium salt is ammonium acetate, ammonium trifluoroacetate, ammonium carbonate, ammonium bicarbonate, or ammonium chloride. In embodiments, the ammonium salt is ammonium acetate. In embodiments of Scheme 4A or 4B, the reaction is performed in the presence of NH₄OAc. In embodiments of Scheme 4A or 4B, the reaction is performed in acetic acid. In embodiments of Scheme 4A or 4B, the reaction is performed at a temperature in the range of about 30° C. to about 150° C., including all values and ranges therebetween. In embodiments of Scheme 4A or 4B, the reaction is performed at a temperature in the range of about 60° C. to about 120° C., including all values and ranges therebetween. In embodiments of Scheme 4A or 4B, the reaction is performed at a temperature in the range of about 80° C. to about 100° C., including all values and ranges therebetween. In embodiments of Scheme 4A or 4B, the reaction is performed at a temperature at about 90° C.

In embodiments, the chiral synthesis of Compounds of formula (II), (IIa) or (IIb), or a pharmaceutically acceptable salt, tautomer, or stereoisomer thereof, comprises a reaction step labeled as Scheme 4C.

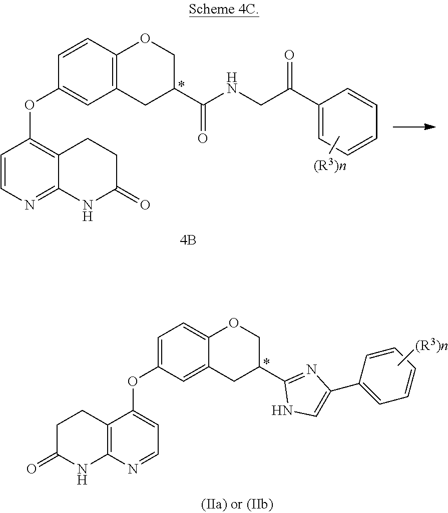

In embodiments of Scheme 4C, a Compound of formula 4B has a (R) or (S) stereochemistry at the position labeled with *. In embodiments of Scheme 4C, a Compound of formula 4B has an enantiomeric excess of at least 85%, at least 90%, or at least 95%.

In embodiments of Scheme 4C, when the stereochemistry of Compound 4B is retained in the product. In embodiments of Scheme 4C, when (S) enantiomer of Compound 4B is used, Compound of formula (IIa) is obtained. In embodiments of Scheme 4C, when (R) enantiomer of Compound 4B is used, Compound of formula (IIb) is obtained.

In embodiments, the chiral purity of a Compound of formula (IIa) prepared by Scheme 4C reaction is within 10% of the chiral purity of an (S) enantiomer of Compound 4B used in the reaction. In embodiments, the chiral purity of a Compound of formula (IIa) prepared by Scheme 4C reaction is within 5% of the chiral purity of an (S) enantiomer of Compound 4B used in the reaction. In embodiments, the chiral purity of a Compound of formula (IIa) prepared by Scheme 4C reaction is greater than 90% when prepared from an (S) enantiomer of Compound 4B having a chiral purity of greater than 90%. In embodiments, the chiral purity of a Compound of formula (IIa) prepared by Scheme 4C reaction is greater than 95% when prepared from an (S) enantiomer of Compound 4B having a chiral purity of greater than 95%. In embodiments, the chiral purity of a Compound of formula (IIa) prepared by Scheme 4C reaction is greater than 98% when prepared from an (S) enantiomer of Compound 4B having a chiral purity of greater than 98%.

In embodiments, the chiral purity of a Compound of formula (IIb) prepared by Scheme 4C reaction is within 10% of the chiral purity of an (R) enantiomer of Compound 4B used in the reaction. In embodiments, the chiral purity of a Compound of formula (IIb) prepared by Scheme 4C reaction is within 5% of the chiral purity of an (R) enantiomer of Compound 4B used in the reaction. In embodiments, the chiral purity of a Compound of formula (IIb) prepared by Scheme 4C reaction is greater than 90% when prepared from an (R) enantiomer of Compound 4B having a chiral purity of greater than 90%. In embodiments, the chiral purity of a Compound of formula (IIb) prepared by Scheme 4C reaction is greater than 95% when prepared from an (R) enantiomer of Compound 4B having a chiral purity of greater than 95%. In embodiments, the chiral purity of a Compound of formula (IIb) prepared by Scheme 4C reaction is greater than 98% when prepared from an (R) enantiomer of Compound 4B having a chiral purity of greater than 98%.

In embodiments of Scheme 4C, the reaction is performed in the presence of ammonia or an ammonium salt. In embodiments, the ammonium salt is ammonium acetate, ammonium trifluoroacetate, ammonium carbonate, ammonium bicarbonate, or ammonium chloride. In embodiments of Scheme 4C, the reaction is performed in the presence of NH₄OAc. In embodiments of Scheme 4C, the reaction is performed in acetic acid. In embodiments of Scheme 4C, the reaction is performed at a temperature in the range of about 30° C. to about 150° C., including all values and ranges therebetween.

In embodiments, the chiral synthesis of Compounds of formula (I), (Ia) or (Ib), or a pharmaceutically acceptable salt, tautomer, or stereoisomer thereof, comprises performing the reaction of Scheme 1 and performing the reaction of Scheme 2A. In embodiments, the chiral synthesis of Compounds of formula (I), (Ia) or (Ib), or a pharmaceutically acceptable salt, tautomer, or stereoisomer thereof, comprises performing the reaction of Scheme 1, Scheme 2A, and Scheme 3A. In embodiments, the chiral synthesis of Compounds of formula (I), (Ia) or (Ib), or a pharmaceutically acceptable salt, tautomer, or stereoisomer thereof, comprises performing the reaction of Scheme 1, Scheme 2A, Scheme 3A, and Scheme 4A.

In embodiments, the chiral synthesis of Compounds of formula (I), (Ia) or (Ib), or a pharmaceutically acceptable salt, tautomer, or stereoisomer thereof, comprising performing one or more of the reaction of Scheme 1, Scheme 2A, Scheme 3A, or Scheme 4A, performing additional reactions before, after, and/or in-between, are not excluded. For example, between the reactions of Scheme 2A and Scheme 3A, another reaction can take place to further functionalize the N-aryl ring, such as a reaction shown below in Scheme 5. Scheme 5 exemplifies a reaction where the substituent R⁶ is further functionalized, within the definition of R⁶.

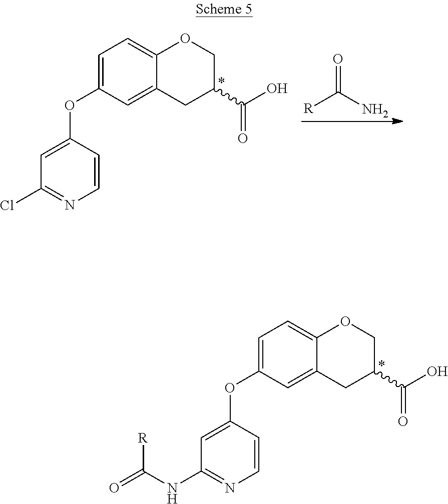

In embodiments, R⁶, R⁷, R⁸, and/or R⁹ in the compound of formula 2A in Scheme 2A is different from R⁶, R⁷, R⁸, and/or R⁹ in the compound of formula 2A in Scheme 3A. In embodiments, R⁶, R⁷, R⁸, and/or R⁹ in the compound of formula 4A in Scheme 3A is different from R⁶, R⁷, R⁸, and/or R⁹ in the compound of formula 4A in Scheme 4A. In embodiments, R¹ in the compound of formula 4A in Scheme 3B is different from R¹ in the compound of formula 4A in Scheme 4B. In embodiments, R³ in the compound of formula 4A in Scheme 3C is different from R³ in the compound of formula 4A in Scheme 4C.

In embodiments, the chiral synthesis of Compounds of formula (I), (Ia) or (Ib), or a pharmaceutically acceptable salt, tautomer, or stereoisomer thereof, comprises performing the reaction of Scheme 1 and performing the reaction of Scheme 2B. In embodiments, the chiral synthesis of Compounds of formula (I), (Ia) or (Ib), or a pharmaceutically acceptable salt, tautomer, or stereoisomer thereof, comprises performing the reaction of Scheme 1, Scheme 2B, and Scheme 3B. In embodiments, the chiral synthesis of Compounds of formula (I), (Ia) or (Ib), or a pharmaceutically acceptable salt, tautomer, or stereoisomer thereof, comprises performing the reaction of Scheme 1, Scheme 2B, Scheme 3B, and Scheme 4B.

In embodiments, the chiral synthesis of Compounds of formula (II), (IIa) or (IIb), or a pharmaceutically acceptable salt, tautomer, or stereoisomer thereof, comprises performing the reaction of Scheme 1 and performing the reaction of Scheme 2B. In embodiments, the chiral synthesis of Compounds of formula (II), (IIa) or (IIb), or a pharmaceutically acceptable salt, tautomer, or stereoisomer thereof, comprises performing the reaction of Scheme 1, Scheme 2B, and Scheme 3C. In embodiments, the chiral synthesis of Compounds of formula (II), (IIa) or (IIb), or a pharmaceutically acceptable salt, tautomer, or stereoisomer thereof, comprises performing the reaction of Scheme 1, Scheme 2B, Scheme 3C, and Scheme 4C.

In embodiments, the chiral synthesis of compounds of formula (I), (Ia), (Ib), (II), (IIa) or (IIb) provides the compound with an enantiomeric excess of at least 85%, at least 90%, at least 95%, or at least 98%.

In embodiments, the chiral synthesis of compounds of formula (I) or (II) provides the compound with (R) or (S) stereochemistry at the carbon marked with a * having greater than: 80% ee, 81% ee, $2% ee, 83% ee, 84% ee, 85% ee, 86% ee, 87% ee, 88% ee, 89% ee, 90% ee, 91% ee, 92% ee, 93% ee, 94% ee, 95% ee, 96% ee, 97% ee, or 98% ee, including all values therebetween.

In embodiments, the chiral synthesis of compounds of formula (Ia), (Ib), (IIa) or (IIb) provides the compound having greater than: 80% ee, 81% ee, 82% ee, 83% ee, 84% ee, 85% ee, 86% ee, 87% ee, 88% ee, 89% ee, 90% ee, 91% ee, 92% ee, 93% ee, 94% ee, 95% ee, 96% ee, 97% ee, or 98% ee, including all values therebetween.

In embodiments, the chiral synthesis as disclosed herein can be used to prepare stereoisomers of compounds disclosed in U.S. Pat. No. 10,183,939, which is hereby incorporated by reference. In embodiments, the compounds disclosed in U.S. Pat. No. 10,183,939 can be prepared as (S) or (R) stereoisomer with the chiral synthesis as disclosed herein. In embodiments, the compounds disclosed in U.S. Pat. No. 10,183,939 can be prepared as (S) or (R) stereoisomer with at least 85% ee, with the chiral synthesis as disclosed herein.

The present disclosure also relates to compounds of formula (I), (Ia), (Ib), (II), (IIa) or (IIb), or pharmaceutically acceptable salt, tautomer, or stereoisomer thereof, prepared according to any one of the methods as disclosed herein.

### Therapeutic Use

The present disclosure also relates to method of using compounds of formula (I), (Ia), (Ib), (II), (IIa) or (IIb), or pharmaceutically acceptable salt, tautomer, or stereoisomer thereof, for treating various diseases and conditions. In embodiments, compounds of formula (I), (Ia), (Ib), (II), (IIa) or (IIb), or pharmaceutically acceptable salt, tautomer, or stereoisomer thereof, are useful for treating a disease or a condition implicated by abnormal activity of one or more Raf kinase. In embodiments, compounds of formula (I), (Ia), (Ib), (II), (IIa) or (IIb), or pharmaceutically acceptable salt, tautomer, or stereoisomer thereof, are useful for treating a disease or a condition treatable by the inhibition of one or more Raf kinase. RAF kinase inhibition is relevant for the treatment of many different diseases associated with the abnormal activity of the MAPK pathway. In embodiments the condition treatable by the inhibition of RAF kinases, such as B-RAF or C-RAF.

In embodiments, the disease or the condition is cancer. In embodiments, the disease or the condition is selected from Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma;
megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancers.

In embodiments, the disease or the condition is melanoma, non-small cell cancer, colorectal cancer, ovarian cancer, thyroid cancer, breast cancer or cholangiocarcinoma. In embodiments, the disease or the condition is colorectal cancer. In embodiments, the disease or the condition is melanoma.

In embodiments, the disease or the condition is cancer comprising a BRAF^{V600E} mutation. In embodiments, the disease or the condition is modulated by BRAF^{V600E}. In embodiments, the disease or the condition is BRAF^{V600E} melanoma, BRAF^{V600E} colorectal cancer, BRAF^{V600E} papillary thyroid cancers, BRAF^{V600E} low grade serous ovarian cancers, BRAF^{V600E} glioma, BRAF^{V600E} hepatobiliary cancers, BRAF^{V600E} hairy cell leukemia, BRAF^{V600E} non-small cell cancer, or BRAF^{V600E} pilocytic astrocytoma.

In embodiments, the disease or the condition is cardio-facio cutaneous syndrome and polycystic kidney disease.

### Pharmaceutical Compositions

The present disclosure also relates to pharmaceutical compositions comprising the compounds of formula (I) or (II), or a pharmaceutically acceptable salt, tautomer, or stereoisomer thereof, and a pharmaceutically acceptable carrier or excipient. The present disclosure also relates to pharmaceutical compositions comprising the compounds of formula (Ia), (Ib), (IIa) or (IIb), or a pharmaceutically acceptable salt, tautomer, or stereoisomer thereof, and a pharmaceutically acceptable carrier or excipient.

In embodiments, the pharmaceutical composition may further comprise an additional pharmaceutically active agent. The additional pharmaceutically active agent may be an anti-tumor agent.

In embodiments, the additional pharmaceutically active agent is an antiproliferative/antineoplastic drug. In embodiments, antiproliferative/antineoplastic drug is alkylating agent (for example cis-platin, oxaliplatin, carboplatin, cyclophosphamide, nitrogen mustard, bendamustin, melphalan, chlorambucil, busulphan, temozolamide and nitrosoureas); antimetabolite (for example gemcitabine and antifolates such as fluoropyrimidines like 5-fluorouracil and tegafur, raltitrexed, methotrexate, pemetrexed, cytosine arabinoside, and hydroxyurea); antibiotic (for example anthracyclines like adriamycin, bleomycin, doxorubicin, daunomycin, epirubicin, idarubicin, mitomycin-C, dactinomycin and mithramycin); antimitotic agent (for example vinca alkaloids like vincristine, vinblastine, vindesine and vinorelbine and taxoids like TAXOL® (paclitaxel) and taxotere and polokinase inhibitors); proteasome inhibitor, for example carfilzomib and bortezomib; interferon therapy; or topoisomerase inhibitor (for example epipodophyllotoxins like etoposide and teniposide, amsacrine, topotecan, mitoxantrone and camptothecin).

In embodiments, the additional pharmaceutically active agent is a cytostatic agent. In embodiments, cytostatic agent is antiestrogen (for example tamoxifen, fulvestrant, toremifene, raloxifene, droloxifene and iodoxyfene), antiandrogen (for example bicalutamide, flutamide, nilutamide and cyproterone acetate), LHRH antagonist or LHRH agonist (for example goserelin, leuprorelin and buserelin), progestogen (for example megestrol acetate), aromatase inhibitor (for example as anastrozole, letrozole, vorazole and exemestane) or inhibitor of 5α-reductase such as finasteride.

In embodiments, the additional pharmaceutically active agent is an anti-invasion agent. In embodiments, the anti-invasion agent is dasatinib and bosutinib (SKI-606), metalloproteinase inhibitor, or inhibitor of urokinase plasminogen activator receptor function or antibody to Heparanase.

In embodiments, the additional pharmaceutically active agent is an inhibitor of growth factor function. In embodiments, the inhibitor of growth factor function is growth factor antibody and growth factor receptor antibody, for example the anti-erbB2 antibody trastuzumab [Herceptin™], the anti-EGFR antibody panitumumab, the anti-erbB1 antibody cetuximab, tyrosine kinase inhibitor, for example inhibitors of the epidermal growth factor family (for example EGFR family tyrosine kinase inhibitor such as gefitinib, erlotinib and 6-acrylamido-N-(3-chloro-4-fluorophenyl)-7-(3-morpholinopropoxy)-quinazolin-4-amine (CI 1033), erbB2 tyrosine kinase inhibitor such as lapatinib); inhibitor of the hepatocyte growth factor family; inhibitor of the insulin growth factor family; modulator of protein regulators of cell apoptosis (for example Bcl-2 inhibitors); inhibitor of the platelet-derived growth factor family such as imatinib and/or nilotinib (AMN107); inhibitor of serine/threonine kinases (for example Ras/RAF signaling inhibitors such as farnesyl transferase inhibitor, for example sorafenib, tipifarnib and lonafarnib), inhibitor of cell signaling through MEK and/or AKT kinase, c-kit inhibitor, abl kinase inhibitor, PI3 kinase inhibitor, Plt3 kinase inhibitor, CSF-1R kinase inhibitor, IGF receptor, kinase inhibitor; aurora kinase inhibitor or cyclin dependent kinase inhibitor such as CDK2 and/or CDK4 inhibitor.

In embodiments, the additional pharmaceutically active agent is an antiangiogenic agent. In embodiments, the antiangiogenic agent inhibits the effects of vascular endothelial growth factor, for example the anti-vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib.

In embodiments, the additional pharmaceutically active agent is a cIn embodiments, the cytotoxic agent is fludaribine (fludara), cladribine, or pentostatin (Nipent™).

In embodiments, the additional pharmaceutically active agent is a steroid. In embodiments, the steroid is corticosteroid, including glucocorticoid and mineralocorticoid, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids as described herein.

In embodiments, the additional pharmaceutically active agent is a targeted therapy agent. In embodiments, the targeted therapy agent is a PI3Kd inhibitor, for example idelalisib and perifosine.

In embodiments, the additional pharmaceutically active agent is an immunotherapeutic agent. In embodiments, the immunotherapeutic agent is antibody therapy agent such as alemtuzumab, rituximab, ibritumomab tiuxetan (Zevalin®) and ofatumumab; interferon such as interferon α; interleukins such as IL-2 (aldesleukin); interleukin inhibitors for example IRAK4 inhibitors; cancer vaccine including prophylactic and treatment vaccines such as HPV vaccines, for example Gardasil, Cervarix, Oncophage and Sipuleucel-T (Provenge); toll-like receptor modulator for example TLR-7 or TLR-9 agonist; and PD-1 antagonist, PDL-1 antagonist, and IDO-1 antagonist.

In embodiments, the pharmaceutical composition may be used in combination with another therapy. In embodiments, the other therapy is gene therapy, including for example approaches to replace aberrant genes such as aberrant p53 or aberrant BRCA1 or BRCA2.

In embodiments, the other therapy is immunotherapy approaches, including for example antibody therapy such as alemtuzumab, rituximab, ibritumomab tiuxetan (Zevalin®) and ofatumumab; interferons such as interferon α; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); toll-like receptor modulators for example TLR-7 or TLR-9 agonists; and PD-1 antagonists, PDL-1 antagonists, and IDO-1 antagonists.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

Pharmaceutical compositions can be prepared as liposome and encapsulation therapeutic agents. For various methods of preparing liposomes and encapsulation of therapeutic agents: see, for example, U.S. Pat. Nos. 3,932,657, 4,311,712, 4,743,449, 4,452,747, 4,830,858, 4,921,757, and 5,013,556. Known methods include the reverse phase evaporation method as described in U.S. Pat. No. 4,235,871. Also, U.S. Pat. No. 4,744,989 covers use of, and methods of preparing, liposomes for improving the efficiency or delivery of therapeutic compounds, drugs and other agents.

Compounds of the invention can be passively or actively loaded into liposomes. Active loading is typically done using a pH (ion) gradient or using encapsulated metal ions, for example, pH gradient loading may be carried out according to methods described in U.S. Pat. Nos. 5,616,341, 5,736,155, 5,785,987, and 5,939,096. Also, liposome loading using metal ions may be carried out according to methods described in U.S. Pat. Nos. 7,238,367, and 7,744,921.

Inclusion of cholesterol in liposomal membranes has been shown to reduce release of drug and/or increase stability after intravenous administration (for example, see: U.S. Pat. Nos. 4,756,910, 5,077,056, and 5,225,212). Inclusion of low cholesterol liposomal membranes continuing charged lipids has been shown to provide cryostability as well as increase circulation after intravenous administration (see: U.S. Pat. No. 8,518,437).

Pharmaceutical compositions can comprise nanoparticles. The formation of nanoparticles has been achieved by various methods. Nanoparticles can be made by precipitating a molecule in a water-miscible solvent, and then drying and pulverizing the precipitate to form nanoparticles. (U.S. Pat. No. 4,726,955). Similar techniques for preparing nanoparticles for pharmaceutical preparations include wet grinding or milling. Other methods include mixing low concentrations of polymers dissolved in a water-miscible solution with an aqueous phase to alter the local charge of the solvent and form a precipitate through conventional mixing techniques. (U.S. Pat. No. 5,766,635). Other methods include the mixing of copolymers in organic solution with an aqueous phase containing a colloid protective agent or a surfactant for reducing surface tension. Other methods of incorporating additive therapeutic agents into nanoparticles for drug delivery require that nanoparticles be treated with a liposome or surfactant before drug administration (U.S. Pat. No. 6,117,454). Nanoparticles can also be made by flash nanoprecipitation (U.S. Pat. No. 8,137,699).

U.S. Pat. No. 7,850,990 covers methods of screening combinations of agents and encapsulating the combinations in delivery vehicles such as liposomes or nanoparticles.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient. The standard duration of treatment with compounds of the invention is expected to vary between one and seven days for most clinical indications. It may be necessary to extend the duration of treatment beyond seven days in instances of recurrent infections or infections associated with tissues or implanted materials to which there is poor blood supply including bones/joints, respiratory tract, endocardium, and dental tissues.

### Examples

As used herein the following terms have the meanings given: “Boc” refers to tert-butyloxycarbonyl; “Cbz” refers to carboxybenzyl; “dba” refers to dibenzylideneacetone; “DCM” refers to dichloromethane; “DIPEA” refers to N,N-diisopropylethylamine; “DMA” refers to dimethylacetamide; “DMF” refers to N,N-dimethylformamide; “DMSO” refers to dimethyl sulfoxide; “dppf” refers to 1,1′-bis(diphenylphosphino)ferrocene; “EtOAc” refers to ethyl acetate; “EtOH” refers to ethanol; “Et₂O” refers to diethyl ether; “IPA” refers to isopropyl alcohol; “LiHMDS” refers to lithium bis(trimethylsilyl)amide; “mCPBA” refers to meta-chloroperoxybenzoic acid; “MeCN” refers to acetonitrile; “MeOH” refers to methanol; “min” refers to minutes; “NMR” refers to nuclear magnetic resonance; “PhMe” refers to toluene; “pTsOH” refers to p-toluenesulfonic acid; “py” refers to pyridine; “r.t.” refers to room temperature; “SCX” refers to strong cation exchange; “T3P” refers to propylphosphonic anhydride; “Tf₂O” refers to trifluoromethanesulfonic anhydride; “THF” refers to tetrahydrofuran; “THP” refers to 2-tetrahydropyranyl; “(UP) LC-MS” refers to (ultra performance) liquid chromatography/mass spectrometry. Solvents, reagents and starting materials were purchased from commercial vendors and used as received unless otherwise described. All reactions were performed at room temperature unless otherwise stated.

In Examples 3, 6 and 7 compound identity and purity confirmations were performed by LC-MS UV using a Waters Acquity SQ Detector 2 (ACQ-SQD2 #LCA081). The diode array detector wavelength was 254 nM and the MS was in positive and negative electrospray mode (m/z: 150-800). A 2 μL aliquot was injected onto a guard column (0.2 μm×2 mm filters) and UPLC column (C18, 50×2.1 mm, <2 μm) in sequence maintained at 40° C. The samples were eluted at a flow rate of 0.6 mL/min with a mobile phase system composed of A (0.1% (v/v) formic acid in water) and B (0.1% (v/v) formic acid in MeCN) according to the gradients outlined below. Retention times RT are reported in minutes.

| | | |
|---|---|---|
| Time (min) | % A | % B |

Final purity

| | | |
|---|---|---|
| 0 | 95 | 5 |
| 1.1 | 95 | 5 |
| 6.1 | 5 | 95 |
| 7 | 5 | 95 |
| 7.5 | 95 | 5 |
| 8 | 95 | 5 |

Short acidic

| | | |
|---|---|---|
| 0 | 95 | 5 |
| 0.3 | 95 | 5 |
| 2 | 5 | 95 |
| 2.6 | 95 | 5 |
| 3 | 95 | 5 |

NMR was also used to characterise final compounds. NMR spectra were obtained on a Bruker AVIII 400 Nanobay with 5 mm BBFO probe. Optionally, compound Rf values on silica thin layer chromatography (TLC) plates were measured. Compound identity and purity confirmations for the remaining examples are described within the example.

Compound purification was performed by flash column chromatography on silica or by preparative LC-MS. LC-MS purification was performed using a Waters 3100 Mass detector in positive and negative electrospray mode (m/z: 150-800) with a Waters 2489 UV/Vis detector. Samples were eluted at a flow rate of 20 mL/min on a Xbridge™ prep C18 5M OBD 19×100 mm column with a mobile phase system composed of A (0.1% (v/v) formic acid in water) and B (0.1% (v/v) formic acid in MeCN) according to the gradient outlined below:

| Time (min) | % A | % B |
|---|---|---|
| 0 | 90 | 10 |
| 1.5 | 90 | 10 |
| 11.7 | 5 | 95 |
| 13.7 | 5 | 95 |
| 14 | 90 | 90 |
| 15 | 90 | 90 |

Chemical names in this document were generated using mol2nam—Structure to Name Conversion by OpenEye Scientific Software. Starting materials were purchased from commercial sources or synthesized according to literature procedures.

The disclosure now being generally described, it will be more readily understood by reference to the following examples which are included merely for purposes of illustration of certain aspects and embodiments of the present invention, and are not intended to limit the invention.

### Example 1. Optimization of Enantioselective Alkene Reduction

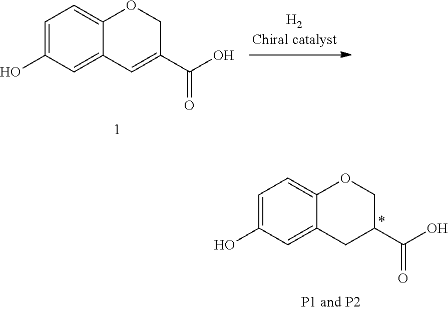 [SMILES: O=C(O)C1=Cc2cc(O)ccc2OC1 ; O=C(O)C1COc2ccc(O)cc2C1]

### General Procedure:

The pre-formed catalysts (4 μmol, substrate/catalyst 25/1) or metal pre-cursors (4 μmol of metal, S/C 25/1) and ligands (4.8 μmol, metal: ligand, 1:1.2) were weighed out into Endeavor vials. The substrate (19.2 mg, 0.1 mmol) was added to each vial as a solution in the specified solvent (2 mL, [S]=0.05 M). If used, triethylamine (14 μL, 0.1 mmol, 1 eq.) was added to the relevant vials. The vials were transferred to an Endeavor, the Endeavor was sealed and set to stir at 650 rpm, purged with nitrogen 5 times, hydrogen 5 times and heated to the specified temperature, at 30 bar H₂. After 16 hours, the Endeavor was vented and purged with nitrogen. About 0.1 mL sample of each reaction was diluted to about 1 mL with MeOH for supercritical fluid chromatography (SFC) analysis. The percentage of each reaction component is measured by integrating all SFC chromatogram peaks and reporting the percentage made up by each component as identified by comparison of retention times of reference samples. The percentage of total peak areas of remaining unidentified peaks are summed together as “Others”. The enantiomeric excess of the major product peak is determined by the peak area ratios of the product peaks in the SFC chromatograms.

### SFC Method

Column: Chiralpak IC-3, 4.6×250 mm, 3 μM
Mobile Phase: A: CO₂; B: 100% methanol
Injection volume: 3 μL
Total time: 10 minutes
Detector: 203 nm
Column temperature 40° C.
Sample diluent: methanol
Flow: 2.0 mL/min

| Time (min) | % A | % B |
|---|---|---|
| 0.00 | 95 | 5 |
| 5.00 | 80 | 20 |
| 7.50 | 50 | 20 |
| 10.00 | 95 | 5 |

### Gradient:

Retention time of starting material (S.M.)=5.6 min
Retention time of first eluting product (P2)=5.8 min
Retention time of second eluting product (P1)=6.1 min

### A. Catalyst Screen

Selected catalysts, which have literature precedence for enantioselective alkene reduction, were tested in typically used solvents: MeOH and THE, and with or without 1 equivalent of triethylamine, which has been shown to aid successful hydrogenation of other acid substrates in this type of reaction (Table 1).

TABLE 1
Catalyst Screen at 70° C.-S/C 25/1, [S] = 0.05M, 70° C., 30 bar H₂, 16 hours

| | | | | S.M. | P2 | P1 | Others | e.e. |
|---|---|---|---|---|---|---|---|---|
| Entry | Catalyst | Solvent | Additive | (%) | (%) | (%) | (%) | (%} |
| 1 | [(S)-BINAP- | MeOH | — | 39 | 49 | 2 | 10 | 91 |
| | RuCl(p-cym)]Cl | | | | | | | |
| 2 | (S)-Phanephos + | MeOH | — | 54 | 8 | 7 | 32 | 8 |
| | [RuCl₂(p-cym.)]₂ | | | | | | | |
| 3 | (R)-MeBoPhoz + | MeOH | — | 2 | 40 | 51 | 7 | 12 |
| | [Rh(COD)₂]OTf | | | | | | | |
| 4 | [(S)-Phanephos | MeOH | — | 0 | 64 | 32 | 5 | 34 |
| | Rh(COD)]BF₄ | | | | | | | |
| 5 | [(S)-BINAP- | MeOH | Net₃ | 0 | 81 | 19 | 0 | 62 |
| | RuCl(p-cym)]Cl | | (1 eq) | | | | | |
| 6 | (S)-Phanephos + | MeOH | Net₃ | 0 | 5 | 93 | 2 | 90 |
| | [RuCl₂(p-cym)]₂ | | (1 eq) | | | | | |
| 7 | (R)-MeBoPhoz + | MeOH | Net₃ | 0 | 28 | 67 | 4 | 41 |
| | [Rh(COD)₂]OTf | | (1 eq) | | | | | |
| 8 | [(S)-Phanephos | MeOH | Net₃ | 0 | 70 | 30 | 0 | 41 |
| | Rh(COD)]BF₄ | | (1 eq.) | | | | | |
| 9 | [(S)-BINAP- | THF | — | 67 | 11 | 22 | 0 | 33 |
| | RuCl(p-cym)]Cl | | | | | | | |
| 10 | (S)-Phanephos + | THF | — | 85 | 11 | 4 | 0 | 49 |
| | [RuCl₂(p-cym)]₂ | | | | | | | |
| 11 | (R)-MeBoPhoz + | THF | — | 15 | 29 | 51 | 6 | 27 |
| | [Rh(COD)₂]OTf | | | | | | | |
| 12 | [(S)-Phanephos | THF | — | 0 | 51 | 49 | 0 | 1 |
| | Rh(COD)]BF₄ | | | | | | | |
| 13 | [(S)-BINAP- | THF | Net₃ | 0 | 56 | 44 | 0 | 13 |
| | RuCl(p-cym)]Cl | | (1 eq.) | | | | | |
| 14 | (S)-Phanephos + | THF | Net₃ | 0 | 31 | 69 | 0 | 37 |
| | [RuCl₂(p-cym)]₂ | | (1 eq.) | | | | | |
| 15 | (R)-MeBoPhoz + | THF | Net₃ | 0 | 33 | 67 | 0 | 35 |
| | [Rh(COD)₂]OTf | | (1 eq.) | | | | | |
| 16 | [(S)-Phanephos | THF | Net₃ | 0 | 54 | 46 | 0 | 7 |
| | Rh(COD)]BF₄ | | (1 eq.) | | | | | |

Entries 1 and 6 in Table 1 resulted in ≥90% ee. In particular entry 6, with (S)-Phanephos and [RuCl₂(p-cym.)]₂, which forms in-situ chiral catalyst, in the presence of triethylamine and methanol solvent provided high conversion (93% P1, 5% P2; total conversion 98%) and high % ee (90%).

In both MeOH and THE, the effect of triethylamine was seen for all catalysts to encourage full conversion. However, in some cases it was also seen to decrease the % ee. The results in MeOH were generally better than in THF.

### B. Solvent and Temperature Screen

The effect of changing the solvent and temperature was tested for the catalyst system in the presence of 1 equiv triethylamine: (S)-Phanephos with [RuCl₂(p-cym)]₂, which was found to give an e.e. of 90% with 98% conversion of product in the initial catalyst screen (Table 1). A background reaction study was carried out with the ligand absent (Table 2, entry 1). This showed that a significant amount of hydrogenation occurred, 70% product, under the ligand-free condition but with very low enantioselectivity. This indicates that it is vital that the chiral ligand-metal complex is formed to achieve the high enantioselectivity. Using a slight excess of ligand (Table 1, entry 6), allowing for a pre-mix of ligand and metal precursor or using a preformed complex can ensure that the chiral ligand-metal complex is formed.

Solvents EtOH and IPA did not appear to give any advantage over MeOH since the results show decreasing % ee values in the order: MeOH, EtOH, IPA (Table 2, comparing entries 2-4 or 5-7).

Decreasing the temperature from 70 to 50° C., gave a slight improvement in the enantioselectivities, while maintaining full conversion. The best result was 93% e.e. obtained in MeOH at 50° C. (entry 5). Decreasing the temperature further to 30° C. showed no further improvement (entry 8).

TABLE 2
Solvent and Temperature Screen with 1 equiv Triethylamine-S/C 25/1,
[S] = 0.05M, 1 eq. NEt₃, 30 bar H₂, 16 hours

| | | | Temp. | S.M. | P2 | P1 | Others | e.e. |
|---|---|---|---|---|---|---|---|---|
| Entry | Catalyst | Solvent | (° C.) | (%) | (%) | (%) | (%) | (%) |
| 1 | [RuCl₂(p-cym)]₂ | MeOH | 70 | 24 | 37 | 33 | 7 | 6 |
| | (no ligand) | | | | | | | |
| 2 | (S)-Phanephos + | MeOH | 70 | 0 | 5 | 92 | 3 | 90 |
| | [RuCl₂(p-cym)]₂ | | | | | | | |
| 3 | (S)-Phanephos + | EtOH | 70 | 0 | 7 | 93 | 0 | 86 |
| | [RuCl₂(p-cym)]₂ | | | | | | | |
| 4 | (S)-Phanephos | IPA | 70 | 0 | 10 | 90 | 0 | 80 |
| | [RuCl₂(p-cym)]₂ | | | | | | | |
| 5 | (S)-Phanephos + | MeOH | 50 | 0 | 4 | 97 | 0 | 93 |
| | [RuCl₂(p-cym)]₂ | | | | | | | |
| 6 | (S)-Phanephos + | EtOH | 50 | 0 | 6 | 94 | 0 | 88 |
| | [RuCl₂(p-cym)]₂ | | | | | | | |
| 7 | (S)-Phanephos + | IPA | 50 | 0 | 8 | 92 | 0 | 84 |
| | [RuCl₂(p-cym)]₂ | | | | | | | |
| 8 | (S)-Phanephos + | MeOH | 30 | 0 | 4 | 96 | 0 | 92 |
| | [RuCl₂(p-cym)]₂ | | | | | | | |

### C. Pre-Formed Catalyst Screen

Two different pre-formed catalysts containing the Phanephos ligand were tested to see whether further improvements to enantioselectivity could be obtained when using a pre-formed catalyst instead of using the ligand and metal precursor in situ (Table 3). The Ru-BINAP pre-formed catalyst was also tested at higher substrate concentrations than previous testing in the initial catalyst screen, which used 0.05 M.

The pre-formed [(R)-Phanephos RuCl₂(p-cym)] catalyst gave a similar result as was obtained from the reaction performed in situ (Table 3, entry 1 can be compared to Table 1, entry 6: 90% e.e.). Thus, there is no apparent improvement with using the preformed version of this ligand-metal combination under these reaction conditions.

The alternative pre-formed catalyst, [(S)-Phanephos Ru(CO)Cl₂(dmf)], which has been found to give improvements to results for similar types of reaction; however, that was not the case with this reaction (entries 2 and 6).

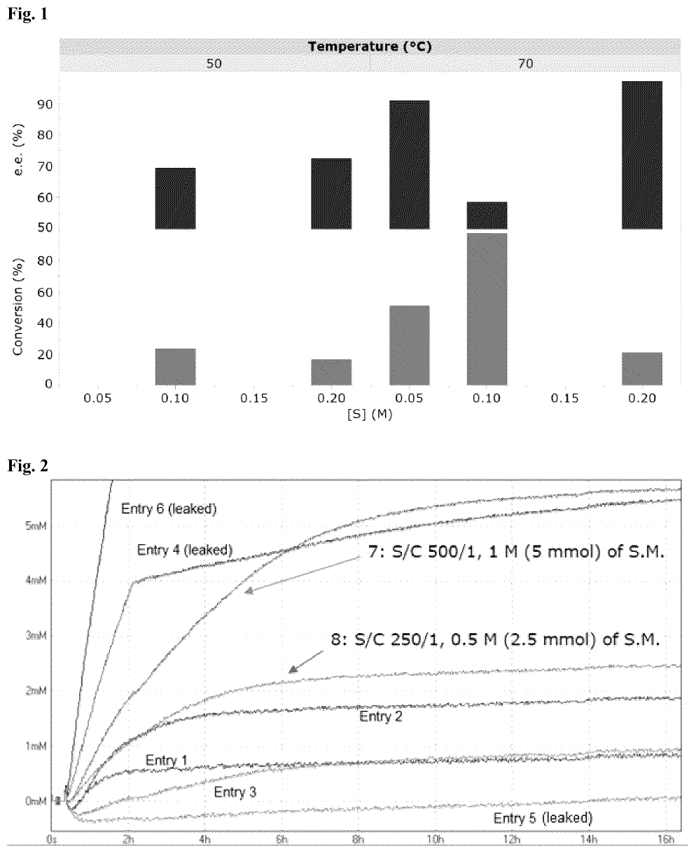

The results from the tests using [(S)-BINAP-RuCl(p-cym)]Cl show there is not a linear trend with regards to the substrate concentration and conversion and enantioselectivity, thus there appears to be a trade-off between achieving high conversion or high e.e. under these conditions (FIG. 1). For example, a very high e.e. of 97% was achieved however the conversion was low with 63% starting material remaining (entry 4). There is uncertainty over the accuracy of this e.e. value however due to an overlap with an impurity. Generally, 70° C. resulted in better conversion and higher e.e. than at 50° C. under these conditions.

TABLE 3
Testing preformed catalysts-S/C 25/1, [S] = 0.05-0.2M, MeOH, 30 bar H₂, 16 hours)

| | | | | Temp. | S.M. | P2 | P1 | Others | |
|---|---|---|---|---|---|---|---|---|---|
| Entry | Catalyst | Additive | [S] | (° C.) | (%) | (%) | (%) | (%) | e.e. |
| 1 | [(R)-Phanephos | Net₃ | 0.05 | 70 | 0 | 95 | 5 | 0 | 89 |
| | RuCl₂(p-cym)] | (1 eq) | | | | | | | |
| 2 | [(S)-Phanephos | Net₃ | 0.05 | 70 | 0 | 42 | 56 | 2 | 14 |
| | Ru(CO)Cl₂(dmf)] | (1 eq) | | | | | | | |
| 3 | [(S)-BINAP- | | 0.1 | 70 | 0 | 77 | 20 | 3 | 59 |
| | RuCl(p-cym)]Cl | | | | | | | | |
| 4 | [(S)-BINAP- | | 0.2 | 70 | 63 | 21 | 0 | 16 | 97 |
| | RuCl(p-cym)]Cl | | | | | | | | |
| 5 | [(R)-Phanephos | Net₃ | 0.05 | 50 | 0 | 93 | 7 | 0 | 86 |
| | RuCl₂p-cym)] | (1 eq.) | | | | | | | |
| 6 | [(S)-Phanephos | Net₃ | 0.05 | 50 | 0 | 39 | 59 | 2 | 20 |
| | Ru(CO)Cl₂(dmf)] | (1 eq.) | | | | | | | |
| 7 | [(S)-BINAP- | | 0.1 | 50 | 76 | 20 | 4 | 0 | 69 |
| | RuCl(p-cym)]Cl | | | | | | | | |
| 8 | [(S)-BINAP- | | 0.2 | 50 | 82 | 14 | 2 | 1 | 72 |
| | RuCl(p-cym)]Cl | | | | | | | | |

D. Ligand Screening with Ruthenium Catalyst

A selection of chiral ligands with varying steric and electronic properties were tested with [RuCl₂(p-cym)]₂ as the precursor, in a small-scale (Table 4A). The ligands (1 μmol) were weighed out into CAT-24 vials. A stock solution of [RuCl₂(p-cym)]₂ (0.83 μmol of metal, S/C 25/1), substrate (21 μmol) and triethylamine (21 μmol, 1 eq.) was made up and 0.25 mL was added to each vial ([S]=0.084 M). A stirrer bar was added to each vial. The CAT-24 was sealed and purged with nitrogen 5 times, hydrogen 5 times (with stirring between each cycle) and set to stir at 800 rpm and heated to 75° C. (internal temperature is estimated to be 5° C. cooler) at 20 bar H2. After 18 hours, the CAT-24 was vented and purged with nitrogen. About 0.1 mL sample of each reaction was diluted to about 1 mL with MeOH to be used for SFC analysis.

All the reactions showed near or complete conversion, thus the ligands can be easily compared. The ligand family which gave the greatest enantioselectivity was Phanephos (entries 5 and 7). The more electron rich variation, An-Phanephos, gave a slight improvement to the e.e. value (entry 7). The e.e. obtained previously using Phanephos and the same Ru precursor was higher (Tables 1 and 2); however, this screen was conducted on a different scale and a different substrate concentration. Another ligand that gave a similarly high e.e. to Phanephos was the Josiphos ligand, SL-J002-1 (entry 10).

TABLE 4A
Ligands Screen for [RuCl₂(p-cym)]₂-S/C 25/1, [S] = 0.08M,
MeOH, 1 eq NEt₃, 70° C., 20 bar H₂, 18 hours

| | Ligand | S.M. | P2 | P1 | Others | e.e. |
|---|---|---|---|---|---|---|
| Entry | (1.2 eq. to Ru) | (%) | (%) | (%) | (%) | (%) |
| 1 | (S)-BINAP | 0 | 66 | 33 | 1 | 33 |
| 2 | (R)-PPhos | 8 | 29 | 56 | 7 | 33 |
| 3 | (S)-Xyl-PPhos | 0 | 80 | 20 | 1 | 60 |
| 4 | (S)-DTBM-Segphos | 0 | 51 | 48 | 1 | 4 |
| 5 | (R)-Phanephos | 0 | 90 | 10 | 0 | 80 |
| 6 | (S)-Xyl-Phanephos | 0 | 20 | 76 | 5 | 58 |
| 7 | (S)-An-Phanephos | 0 | 8 | 88 | 4 | 84 |
| 8 | (R)-MeBoPhoz | 0 | 43 | 53 | 4 | 10 |
| 9 | (S)-H8Binol-BoPhoz | 2 | 46 | 36 | 16 | 12 |
| 10 | Josiphos SL-J002-1 | 0 | 10 | 80 | 10 | 77 |
| | (Ph/tBu) | | | | | |
| 11 | Josiphos SL-J001-1 | 0 | 34 | 62 | 4 | 29 |
| | (Ph/CY) | | | | | |
| 12 | Josiphos SL-J003-2 | 0 | 58 | 41 | 1 | 17 |
| | (Cy/Cy) | | | | | |
| 13 | Mandyphos SL-M002-2 | 0 | 47 | 51 | 2 | 3 |
| | (Cy) | | | | | |
| 14 | (S,S)-Me-DuPhos | 0 | 76 | 23 | 1 | 54 |
| 15 | (S,S)-iPr-DuPhos | 0 | 15 | 80 | 5 | 69 |
| 16 | (S,S)-BDPP | 0 | 29 | 65 | 6 | 39 |
| 17 | (R,R)-Ph-BPE | 0 | 49 | 49 | 2 | 0 |
| 18 | (R)-H8-BINAP | 0 | 13 | 82 | 5 | 73 |
| 19 | (5,5)-Norphos | 0 | 69 | 31 | 1 | 38 |
| 20 | (S)-Prophos | 0 | 34 | 63 | 4 | 30 |
| 21 | (S,S)-DIOP | 0 | 46 | 52 | 2 | 6 |
| 22 | (R,R)-BPPM | 0 | 43 | 55 | 2 | 12 |
| 23 | (S,S)-PPM | 1 | 33 | 61 | 4 | 30 |

In addition, two different pre-formed Ru-BINAP catalysts were tested in MeOH or 2,2,2-trifluoroethanol (TFE) and with the addition of an alternative, more sterically demanding, base than the previously tested—e.g., triethylamine (Table 4B). Appropriate amounts of catalyst (8 μmol, S/C 50/1) and substrate (76.8 mg, 0.4 mmol, 0.2 M) were weighed out into Endeavor vials. The solvent (2 mL) was added followed by N,N-diisopropylethylamine (69 μL, 0.4 mmol, 1 eq.) for appropriate vials. The vials were transferred to an Endeavor, the Endeavor was sealed and set to stir at 650 rpm, purged with nitrogen 5 times, hydrogen 5 times and heated to 70° C. at 30 bar H2. After 16 hours, the Endeavor was vented and purged with nitrogen. About 0.1 mL sample of each reaction was diluted to about 1 mL with MeOH for SFC analysis.

TFE gave significantly lower conversions and lower e.e. values than in MeOH (entries 5-6 compared with 1-2). The addition of N (iPr)₂Et (Hunig's base) gave an improvement in conversion with the [(S)-BINAP-RuCl(p-cym)]Cl catalyst however obtained a lower e.e. (entry 3 compared with 1). The same effect was previously observed when testing triethylamine as an additive (Table 1).

TABLE 4B
Screening of Pre-formed Ru-BINAP catalysts-S/C 50/1,
[S] = 0.2M, MeOH, 70° C., 30 bar H₂, 16 hours

| | | | Base | S.M. | P2 | P1 | Others | e.e. |
|---|---|---|---|---|---|---|---|---|
| Entry | Catalyst | Solvent | (1 eq) | (%) | (%) | (%) | (%) | (%) |
| 1 | [(S)-BINAP- | MeOH | — | 65 | 26 | 0 | 9 | 97 |
| | RuCl(p- [symbol] | | | | | | | |
| 2 | (R)-BINAP | MeOH | — | 0 | 18 | 76 | 6 | 62 |
| | Ru(OAc)₂ | | | | | | | |
| 3 | [(S)-BINAP- | MeOH | N(iPr)₂Et | 0 | 81 | 19 | 0 | 62 |
| | RuCl(p- [symbol] | | | | | | | |
| 4 | (R)-BINAP | MeOH | N(iPr)₂Et | 0 | 16 | 78 | 6 | 66 |
| | Ru(OAc)₂ | | | | | | | |
| 5 | [(S)-BINAP- | TFE | — | 85 | 14 | 2 | 0 | 77 |
| | RuCl(p- [symbol] | | | | | | | |
| 6 | (R)-BINAP | TFE | — | 46 | 20 | 34 | 0 | 26 |
| | Ru(OAc)₂ | | | | | | | |

[symbol]

indicates data missing or illegible when filed

E. Ligand Screening with Rhodium Catalyst

A selection of chiral ligands with varying steric and electronic properties were tested with [Rh(COD)₂]OTf as the precursor, in a small-scale as discussed for ligand screening with ruthenium catalyst (Table 5). Each ligand was tested in the absence and presence of 1 equivalent of triethylamine, with respect to substrate.

The majority of the reactions showed full consumption of the starting material, indicating that ligand to metal complexation had occurred. The reactions in the presence of triethylamine generally gave lower e.e. value than obtained in the absence of triethylamine. However, triethylamine also gave results with significantly lower amounts of side-product than the reactions without triethylamine. One unidentified side-product which appeared in large amounts in some reactions had a retention time of 6.4 minutes by SFC.

(R)-Phanephos and (S)-Xyl-Phanephos were found to give very high e.e. values in absence of triethylamine. However, the amount of the unknown side-product (at 6.4 min) was also very high in these reactions (entries 4-5). It also seems unlikely that opposite enantiomers of these ligands would form the same enantiomer of product preferentially, as it appears to have done in entries 4-5, thus the presence of side-products may be affecting the ratio of the observed peaks in the chromatograms.

TABLE 5
Screening Ligands with [Rh(COD)₂]OTf-S/C 25/1,
[S] = 0.08M, MeOH, 70° C., 20 bar H₂, 16 hours

| | Ligand | | S.M. | P2 | P1 | Others | e.e. |
|---|---|---|---|---|---|---|---|
| Entry | (1.2 eq. to Rh) | Additive | (%) | (%) | (%) | (%) | (%) |
| 1 | (S)-BINAP | — | 7 | 30 | 6 | 58 | 68 |
| 2 | (R)-PPhos | — | 0 | 53 | 4 | 43 | 88 |
| 3 | (S)-Xyl-PPhos | — | 0 | 41 | 2 | 57 | 91 |
| 4 | (R)-Phanephos | | 0 | 35 | 1 | 65 | ≤97* |
| 5 | (S)-Xyl-Phanephos | — | 0 | 58 | 0 | 42 | ≤99* |
| 6 | (R)-MeBoPhoz | — | 1 | 35 | 39 | 25 | 6 |
| 7 | (S)-H8Binol-BoPhoz | — | 33 | 5 | 2 | 60 | 47 |
| 8 | Josiphos SL-J002-1 | — | 0 | 30 | 32 | 38 | 2 |
| | (Ph/tBu) | | | | | | |
| 9 | (S,S)-Me-DuPhos | — | 0 | 42 | 27 | 30 | 22 |
| 10 | (S,S)-iPr-DuPhos | — | 0 | 45 | 21 | 33 | 36 |
| 11 | (S,S)-Norphos | — | 0 | 49 | 5 | 46 | 81 |
| 12 | (R,R)-BPPM | — | 0 | 36 | 6 | 59 | 73 |
| 13 | (S)-BINAP | Net₃ | 1 | 37 | 52 | 10 | 18 |
| | | (1 eq.) | | | | | |
| 14 | (R)-PPhos | Net₃ | 1 | 54 | 42 | 3 | 13 |
| | | (1 eq.) | | | | | |
| 15 | (S)-Xyl-PPhos | Net₃ | 2 | 44 | 50 | 4 | 6 |
| | | (1 eq.) | | | | | |
| 16 | (R)-Phanephos | Net₃ | 0 | 16 | 75 | 9 | 65 |
| | | (1 eq.) | | | | | |
| 17 | (S)-Xyl-Phanephos | Net₃ | 0 | 72 | 27 | 1 | 45 |
| | | (1 eq.) | | | | | |
| 18 | (R)-MeBoPhoz | Net₃ | 1 | 34 | 58 | 7 | 26 |
| | | (1 eq.) | | | | | |
| 19 | (S)-H8Binol-BoPhoz | Net₃ | 13 | 38 | 17 | 32 | 39 |
| | | (1 eq.) | | | | | |
| 20 | Josiphos SL-J002-1 | Net₃ | 0 | 46 | 51 | 3 | 5 |
| | (Ph/tBu) | (1 eq.) | | | | | |
| 21 | (S,S)-Me-DuPhos | Net₃ | 0 | 35 | 60 | 6 | 27 |
| | | (1 eq.) | | | | | |
| 22 | (S,S)-iPr-DuPhos | Net₃ | 0 | 43 | 54 | 3 | 12 |
| | | (1 eq.) | | | | | |
| 23 | (S,S)-Norphos | Net₃ | 0 | 53 | 45 | 2 | 7 |
| | | (1 eq.) | | | | | |
| 24 | (R,R)-BPPM | Net₃ | 1 | 30 | 63 | 6 | 35 |
| | | (1 eq.) | | | | | |

To assess whether the unknown side product (at 6.4 min) was derived from the substrate (compound 1) or the product (P1 and P2), stability of the substrate and the products were studied (Table 6). Compound 1 or racemic product (0.4 mmol) was weighed out into Endeavor vials. MeOH (2 mL) was added to each vial. The vials were transferred to an Endeavor, the Endeavor was sealed and set to stir at 650 rpm, purged with nitrogen 5 times, hydrogen 5 times and heated to 50 or 90° C. at 30 bar H2. After 16 or 56 hours, the Endeavor was vented and purged with nitrogen. About 0.1 mL sample of each reaction was diluted to about 1 mL with MeOH for SFC analysis

Heating the substrate at 90° C. for 16 hours did not cause any change in the SFC chromatogram (entries 1 and 3). Heating the racemic product sample, however, showed a reduction in the second eluting product peak (P1) and the significant increase in the side-product appearing at 6.4 minutes in the SFC chromatogram, increase from 2% to 16% (entries 2 and 4). Heating the product at 90° C. for a longer time showed a further increase in the amount of this side-product (entry 6). Heating at 50° C. gave a smaller amount of this side-product (entry 5). It therefore seems that higher temperature and the presence of acid encourages this side-product to form (lower temperature and presence of base can suppress it as found during previous reactions).

TABLE 6
Stability of Compound 1 and Racemic Product (P1/P2)-
[S] = 0.2M, MeOH, 50-90° C., 30 bar H₂, 16-56 hours

| | S.M. or | Temp. | Time | S.M. | P2 | P1 | Others | e.e. |
|---|---|---|---|---|---|---|---|---|
| Entry | Prod. | (° C.) | (h) | (%) | (%) | (%) | (%) | (%) |
| 1 | S.M. | — | — | 100 | — | — | 0 | — |
| 2 | Rac-Prod. | — | — | — | 47 | 50 | 3 | 3 |
| 3 | S.M. | 90 | 16 | 100 | — | — | 0 | — |
| 4 | Rac-Prod. | 90 | 16 | — | 47 | 37 | 17 | 12 |
| 5 | Rac-Prod. | 50 | 56 | — | 48 | 42 | 10 | 6 |
| 6 | Rac-Prod. | 90 | 56 | — | 48 | 28 | 24 | 26 |

Because the results of the ligand screen with [Rh(COD)₂]OTf showed Phanephos as giving 97% e.e., albeit with 65% of “others” in the SFC chromatogram (Table 5), two different preformed Rₕ-Phanephos catalysts were tested in different solvents and temperatures (Table 7). Appropriate amounts of catalyst (8 μmol, S/C 50/1) and substrate (76.8 mg, 0.4 mmol, 0.2 M) were weighed out into Endeavor vials. The solvent (2 mL) was added into each vial. The vials were transferred to an Endeavor, the Endeavor was sealed and set to stir at 650 rpm, purged with nitrogen 5 times, hydrogen 5 times and heated to 50 or 70° C. at 30 bar H2. After 16 hours, the Endeavor was vented and purged with nitrogen. About 0.1 mL sample of each reaction was diluted to about 1 mL with MeOH for SFC analysis.

The results show that the amount of “others” seems to depend mostly on the temperature and also on the catalyst used. The least amount of “others” was obtained with [(S)-Phanephos Rh(COD)]BF₄ catalyst compared to [(S)-Phanephos Rh(COD)]OTf under all the conditions tested. The e.e. values obtained (Table 7) were lower than those obtained in the smaller scale ligand screen (Table 5). Because the major product appeared to be the first eluting peak (P2) in both cases, when opposite ligand enantiomers were used, this indicates that there may be a side-product which co-elutes with the first eluting product peak (5.8 min) which is therefore interfering with the calculated e.e. values. Thus, the results in Table 7 are likely to have lower e.e. values than have been calculated by using the relative integration of the peaks at 5.8 min (P2) and 6.1 min (P1). The reactions in ethanol are more likely to have a more accurate e.e. values as the side-products have better separation from the product peaks. The side-products from the reactions in ethanol appear at slightly different retention times than the reactions in methanol (see Tables 8A and 8B). NMR analysis suggests that the side-products are the methyl esters or ethyl esters (of both enantiomers of product) for the reactions in methanol or ethanol respectively.

TABLE 7
Screening of Rh-Phanephos catalysts under different conditions-S/C 50/1,
[S] = 0.2M, MeOH, 50-70° C., 30 bar H₂, 16 hours

| | | | Temp. | S.M. | P2 | P1 | Others | e.e. |
|---|---|---|---|---|---|---|---|---|
| Entry | Catalyst | Solvent | (° C.) | (%) | (%) | (%) | (%) | (%) |
| 1 | [(S)-Phanephos | MeOH | 50 | 0 | 67 | 21 | 12 | 52 |
| | Rh(COD)]BF₄ | | | | | | | |
| 2 | [(S)-Phanephos | MeOH | 50 | 0 | 47 | 24 | 29 | 31 |
| | Rh(COD)]OTf | | | | | | | |
| 3 | [(S)-Phanephos | EtOH | 50 | 0 | 65 | 25 | 9 | 44 |
| | Rh(COD)]BF₄ | | | | | | | |
| 4 | [(S)-Phanephos | EtOH | 50 | 0 | 26 | 23 | 50 | 6 |
| | Rh(COD)]OTf | | | | | | | |
| 5 | [(S)-Phanephos | EtOH | 70 | 0 | 58 | 21 | 21 | 46 |
| | Rh(COD)]BF₄ | | | | | | | |
| 6 | [(S)-Phanephos | EtOH | 70 | 0 | 6 | 5 | 89 | 17 |
| | Rh(COD)]OTf | | | | | | | |

TABLE 8A
SFC Readout of Table 7, Entry 2 (MeOH)

| | Peak Name | RT | Area | % Area | Height |
|---|---|---|---|---|---|
| 1 | | 5.453 | 74133 | 2.52 | 17977 |
| 2 | SM | 5.600 | | | |
| 3 | | 5.734 | 95521 | 3.25 | 25732 |
| 4 | P2 | 5.842 | 1373483 | 46.76 | 268748 |
| 5 | P1 | 6.151 | 716744 | 24.40 | 110218 |
| 6 | | 6.398 | 677709 | 23.07 | 186998 |

TABLE 8B
SFC Readout of Table 7, Entry 6 (EtOH)

| | Peak Name | RT | Area | % Area | Height |
|---|---|---|---|---|---|
| 1 | | 5.341 | 81971 | 2.15 | 27880 |
| 2 | SM | 5.600 | | | |
| 3 | | 5.729 | 1589281 | 41.76 | 526310 |
| 4 | P2 | 5.860 | 241850 | 6.35 | 40341 |
| 5 | P1 | 6.164 | 172907 | 4.54 | 35584 |
| 6 | | 6.294 | 1720143 | 45.19 | 410417 |

### F. Catalyst Loading Screening

(S)-Phanephos and [RuCl₂(p-cym)]₂ combination was tested at lower catalyst loadings and higher substrate concentrations (Table 9). For entries 1-8: Appropriate amounts of substrate (19.2 mg, 0.1 mmol for 0.05 M, 38.4 mg, 0.2 mmol, 0.1 M or 76.8 mg, 0.4 mmol, 0.2 M) were weighed out into Endeavor vials. A stock solution of (S)-Phanephos and [RuCl₂(p-cym)]₂ (1.2:1 eq.) was made in MeOH and appropriate volumes were added to each vial. More MeOH was added to each vial to make the total volume of MeOH equal to 2 mL. Triethylamine (1 eq.) was added to each vial. The vials were transferred to an Endeavor, the Endeavor was sealed and set to stir at 650 rpm, purged with nitrogen 5 times, hydrogen 5 times and heated to 50° C. at 30 bar H₂. After 16 hours, the Endeavor was vented and purged with nitrogen. About 0.1 mL sample of each reaction was diluted to about 1 mL with MeOH for SFC analysis. For entries 9-11: Same procedure as above but with larger amounts reagents: (S)-Phanephos and [RuCl₂(p-cym)]₂ (1.2:1 eq., 2.9 mg, 1.2 mg), substrate (192 mg, 1 mmol), NEt₃ (140 μL, 1 mmol, 1 eq.) and 5 mL MeOH.

All the reactions (entries 1-8) gave full conversion and 91-92% e.e. values. This shows that there was no impact on the reactions by decreasing the catalyst loading to S/C 200/1 (0.5 mol %) and by increasing the substrate concentration to 0.2 M.

A few reactions were carried out on a slightly larger scale (still in the Endeavor), to verify these good results at S/C 200/1. Two repeats gave the same result, full conversion with 90% e.e. (entries 9-10). The background reaction of the metal precursor and substrate was tested, at 200/1 metal/substrate loading. The conversion of hydrogenated product was significantly lower than when previously tested using 25/1 loading which gave 70% product compared to the 17% obtained in this case (entry 11). This demonstrates that there is ligand accelerated catalysis when Phanephos has bonded to the metal to make the chiral complex. It also suggests that lower loadings may help to eliminate the possibility of non-selective hydrogenation carried out by any unreacted metal precursor complex.

TABLE 9
Catalyst Loading and Substrate Concentration
Screening-S/C 50/1-200/1, [S] = 0.05-0.2M,
MeOH, 1 eq. NEt₃, 50° C., 20 bar H₂, 16 hours

| | Catalyst | | | | | | |
|---|---|---|---|---|---|---|---|
| | Loading | [S] | S.M. | P2 | P1 | Others | e.e. |
| Entry | (S/C) | (M) | (%) | (%) | (%) | (%) | (%) |
| 1 | 50/1 | 0.05 | 0 | 5 | 96 | 0 | 91 |
| 2 | 50/1 | 0.10 | 0 | 4 | 96 | 0 | 92 |
| 3 | 100/1 | 0.05 | 0 | 4 | 96 | 0 | 91 |
| 4 | 100/1 | 0.10 | 0 | 4 | 96 | 0 | 92 |
| 5 | 100/1 | 0.20 | 0 | 4 | 96 | 0 | 92 |
| 6 | 200/1 | 0.05 | 0 | 5 | 96 | 0 | 91 |
| 7 | 200/1 | 0.10 | 0 | 5 | 96 | 0 | 91 |
| 8 | 200/1 | 0.20 | 0 | 4 | 96 | 0 | 91 |

1 mmol substrate scale reactions (5 mL MeOH)

| | | | | | | | |
|---|---|---|---|---|---|---|---|
| 9 | 200/1 | 0.20 | 0 | 5 | 95 | 0 | 90 |
| 10 | 200/1 | 0.20 | 0 | 5 | 95 | 0 | 90 |
| 11 | 200/1 | 0.20 | 83 | 10 | 7 | 0 | 20 |
| | (no ligand) | | | | | | |

*Entry 4 had 2 eq. of NEt₃.

In summary, the screening experiments found MeOH to give the best results in terms of conversion and enantioselectivity. The addition of 1 equivalent of triethylamine was found to improve results with certain catalyst systems, such as making it possible to achieve ≥90% e.e. with ≥98% product. This was obtained with (S)-Phanephos and [RuCl₂(pcym)]₂.

The ligand screen with Ru identified (S)-Phanephos and (S)-An-Phanephos to give the best results. Some tests with preformed Ru-Phanephos catalysts gave no improvement to the results obtained using the ligand and metal precursor in situ. The loading of (S)-Phanephos and [RuCl₂(p-cym)]₂ catalyst system was decreased to S/C 200/1 and was shown to still give full conversion and 90% e.e. of product. Increasing the concentration to 0.2 M was also demonstrated to have no effect on the outcome of the results.

Reactions using rhodium-based catalysts were generally found to give very high amounts of side-product. The major side-product was decreased in the presence of triethylamine. However, low e.e. values were also obtained under those conditions. The major side-product from these reactions has been tentatively assigned, by NMR analysis, as the methyl ester of the saturated product when the reaction is carried out in methanol or the ethyl ester for a reaction in ethanol.

Also, decreasing the temperature from 70° C. to 50° C. encouraged a slight improvement on e.e. from 90 to 93%. Decreasing to 30° C. gave no further improvement.

### Example 2. Further Optimization of Enantioselective Alkene Reduction

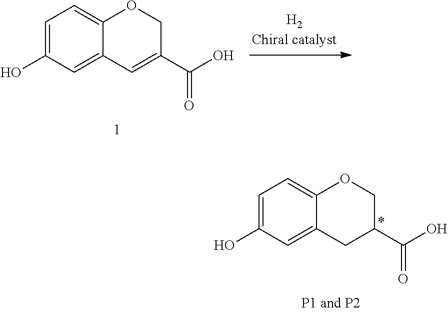 [SMILES: O=C(O)C1=Cc2cc(O)ccc2OC1 ; O=C(O)C1COc2ccc(O)cc2C1]

Material and Methods: SFC method described in Example 1 was used.

Example 1 identified Phanephos and [RuCl₂(p-cym)]₂ catalyst system as being one of the best in obtaining high conversion and high % ee of the product. This study was undertaken to further optimize the reaction conditions for Phanephos and [RuCl₂(p-cym)]₂ catalyst system.

### A. Catalyst Loading and Substrate Concentration

In Example 1 it was found that the catalyst loading can be reduced from S/C 25/1 to S/C 200/1 and the substrate concentration can be increased from 0.05 M to 0.2 M. Across those ranges tested in Example 1, there was no decrease in conversion or enantioselectivity, with full conversion and ≥90% e.e. obtained at S/C 200/1 and 0.2 M substrate concentration.

Further catalyst loading and substrate concentration study was performed. A stock solution of (R)-Phanephos and [RuCl₂(p-cym)]₂ (1.2:1 eq.) was made in DCM for the reactions using S/C 1,000/1 or 10,000/1 and appropriate volumes of the solution was added to those vials before the DCM was blown off with N₂. (R)-Phanephos and [RuCl₂(p-cym)]₂ (1.2:1 eq.) was weighed out into the vials for catalyst loadings 200/1 to 500/1. Appropriate amounts of substrate (i.e. 192 mg, 1 mmol) was weighed out into Endeavor vials. Methanol (2 mL for entries 1-6 and 5 mL for 7-8; Table 10) was added into each vial followed by triethylamine (1 eq.). The vials were transferred to an Endeavor, the Endeavor was sealed and set to stir at 650 rpm, purged with nitrogen 5 times, hydrogen 5 times and heated to 50° C. at 30 bar H2. After 16 hours, the Endeavor was vented and purged with nitrogen. About 0.1 mL sample of each reaction was diluted to about 1 mL with MeOH for SFC analysis (Table 10). The hydrogen uptake time is approximated from the data recorded by the Endeavor which shows at what time the uptake has stopped, therefore the reaction is assumed to be ≥90% complete at this point. There was a leak in the Endeavor for entries 4-6 so the uptake was not recorded accurately.

Decreasing the catalyst loading further showed S/C 1,000/1 to give full conversion (entry 3), whereas S/C 10,000/1 gave only ≤15% of hydrogenation product, after a 16-hour reaction (entries 5-6). Lower catalyst loadings were also found to give slightly lower e.e. values. However, increasing the substrate concentration was shown to have a larger effect on decreasing the enantioselectivities (entries 1-2).

By looking at the hydrogen uptakes recorded from the Endeavor software, an approximate time at which the reaction is likely to be ≥90% complete was deduced (FIG. 2). Thus, the increase in substrate concentration from 0.5 M to 1 M is shown to significantly affect the reaction rate such that at S/C 200/1, a reaction with 0.5 M concentration took approximately 2 hours for the H2 consumption to stop while 1 M took approximately 5 hours (FIG. 2, compare entries 1 and 2, which corresponds to entries 1 and 2 of Table 10). As expected, decreasing the catalyst loading also decreased the reaction rate, thus S/C 1,000/1 reached completion in approximately 10 hours (FIG. 2, entry 3).

TABLE 10
Catalyst Loading Screen for (R)-Phanephos and [RuCl₂(p-cym)]₂
and Substrate Concentration Study-S/C 200/1-10,000/1,
[S] = 0.5-1.0M, MeOH, 1 eq. NEt₃, 50° C., 30 bar H₂, 16 hours

| | Catalyst | | H₂ | | | | | |
|---|---|---|---|---|---|---|---|---|
| | Loading | [S] | Uptake Time | S.M. | P2 | P1 | Others | e.e. |
| Entry | (S/C) | (M) | (h) | (%) | (%) | (%) | (%) | (%) |
| 1 | 200/1 | 0.5 | 2 | 0 | 94 | 6 | 0 | 88 |
| 2 | 200/1 | 1 | 5 | 0 | 90 | 10 | 0 | 80 |
| 3 | 1,000/1 | 0.5 | 10 | 1 | 91 | 8 | 0 | 84 |
| 4 | 1,000/1 | 1 | n.d. | 9 | 81 | 9 | 1 | 80 |
| 5 | 10,000/1 | 0.5 | n.d. | 85 | 11 | 4 | 0 | n.d |
| 6 | 10,000/1 | 1 | n.d. | 91 | 8 | 1 | 0 | n.d |
| 7 | 500/1 | 1 | 14 | 0 | 91 | 9 | 0 | 82 |
| 8 | 250/1 | 0.5 | 8 | 0 | 92 | 8 | 0 | 84 |

### B. Kinetic Analysis Hydrogenation Reaction

In order to investigate the reasons behind any difficulty in being able to minimize the catalyst loading, some kinetic analysis was carried out. The hydrogen uptake data recorded by the Endeavor was able to be transformed into consumption rates of the starting material. Kinetic analysis of reactions using the same catalyst concentration, but different initial starting material concentrations was performed. This followed the method used to distinguish whether there is any product inhibition or catalyst deactivation, termed Variable Time Normalisation Analysis (VTNA) in Nielsen, et. al. Chem. Sci., 2019, 10, 348.

(R)-Phanephos and [RuCl₂(p-cym)]₂ (1.2:1 eq, 7 mg and 3.1 mg respectively) was weighed out into Endeavor vials. Different amounts of substrate (i.e. 480 mg, 2.5 mmol) were weighed out into Endeavor vials to make the required substrate concentrations. Methanol (5 mL) was added into each vial followed by triethylamine (1 eq.). The vials were transferred to an Endeavor, the Endeavor was sealed and set to stir at 650 rpm, purged with nitrogen 5 times, hydrogen 5 times and heated to 50° C. at 30 bar H2. After 16 hours, the Endeavor was vented and purged with nitrogen. About 0.1 mL sample of each reaction was diluted to about 1 mL with MeOH for SFC analysis. The hydrogen uptake time is approximated from the data recorded by the Endeavor which shows at what time the uptake has stopped, therefore the reaction is assumed to be >90% complete at this point.

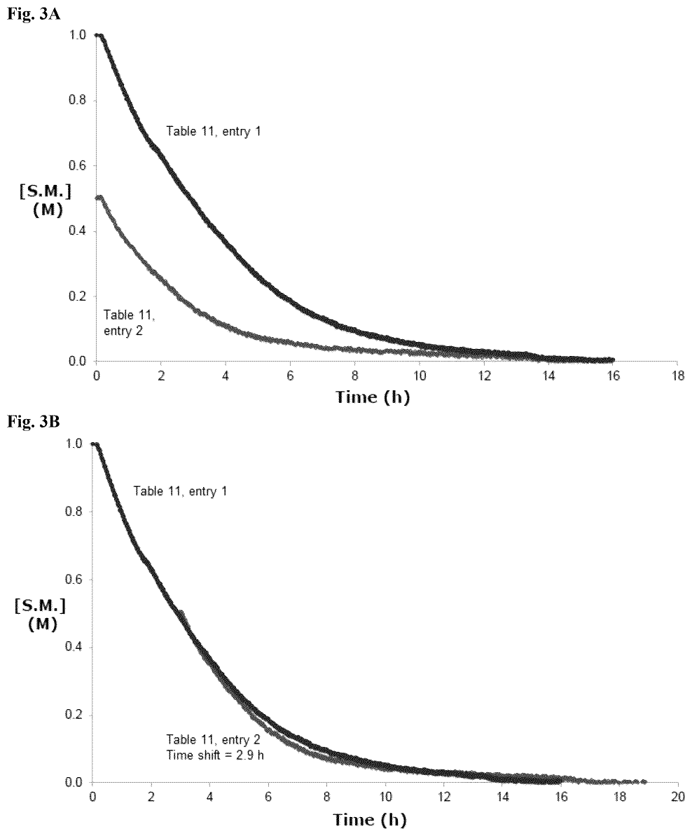

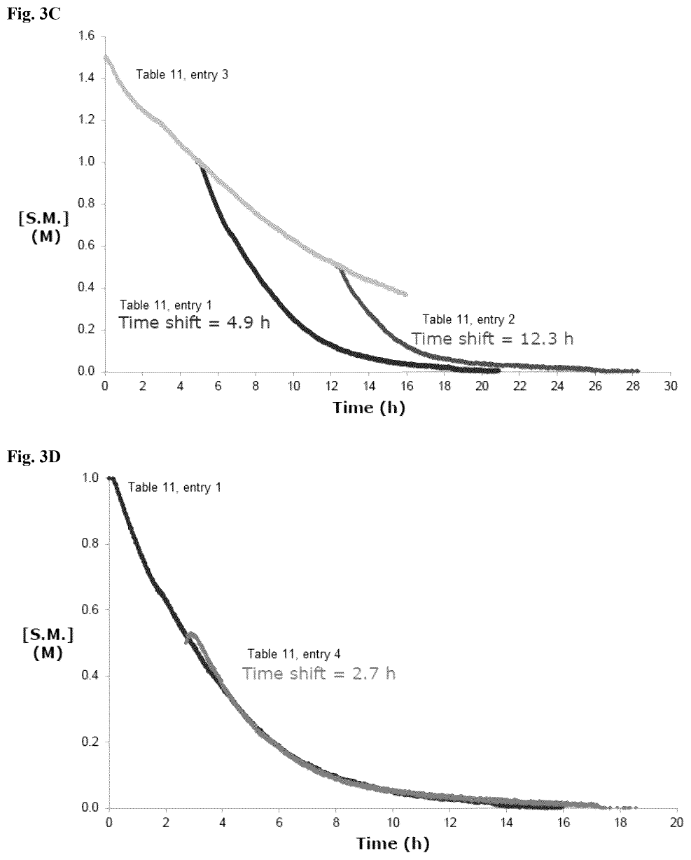

The reaction curves of the first two reactions, with 1.0 or 0.5 M substrate concentration (Table 11, entries 1-2), were overlaid on the same graph (FIG. 3A). The reaction with the lower starting concentration of substrate (entry 2) was then shifted in time (to the right) so that the first data point lined up with the higher substrate concentration reaction (FIG. 3B). The reaction curves appear to be very similar once they are overlaid by shifting the lower concentration reaction in time by 2.9 hours (FIG. 3B). This is suggestive of a lack of product inhibition or catalyst deactivation, as per the logic of VTNA.

A third experiment was then carried out using an even higher substrate concentration (Table 11, entry 3). It is worth noting that this reaction did not reach completion within the 16-hour reaction timeframe. The reaction curves for these three reactions were overlaid on the same graph by shifting the reactions with the lower concentrations onto this higher concentration reaction (FIG. 3C). As shown in FIG. 3C, the reaction curves did not overlap. Thus, this suggests some differences arise at this increased concentration (Table 11, entry 3) which effect the catalysis.

To distinguish between whether catalyst deactivation or product inhibition was the most likely cause of the effects with increased substrate concentration and catalyst loading, a final experiment was carried out where 0.5 M of racemic product was added into the starting mixture (Table 11, entry 4). The presence of the overlap of the curves in FIG. 3D (Table 11 entries 1 and 4) suggests that any difference in rate between the reactions at different substrate concentrations may be due to some product inhibition and not catalyst deactivation. It is worth noting that in these reactions with different substrate concentrations, although the amount of triethylamine is kept as 1 molar equivalent with respect to substrate, the pH will be different in each reaction, which may be affecting the catalysis and thus this analysis of the reaction kinetics. However, this is unlikely to influence the main finding of this analysis: up to a substrate concentration of 1.0 M, any product inhibition or catalyst deactivation should be insignificant. This means that it should be possible to use low catalyst loadings and obtain good results.

TABLE 11
Kinetic Analysis Study-S/C 250/1-750/1, [S] = 0.5-1.5M,
MeOH, 1 eq. NEt3, 50° C., 30 bar H₂, 16 hours

| | | | H₂ | | | | | |
|---|---|---|---|---|---|---|---|---|
| | Catalyst | | Uptake | | | | | |
| | Loading | [S] | Time | S.M. | P2 | P1 | Others | e.e. |
| Entry | (S/C) | (M) | (h) | (%) | (%) | (%) | (%) | (%) |
| 1 | 500/1 | 1.0 | 14 | 0 | 91 | 9 | 0 | 82 |
| 2 | 250/1 | 0.5 | 8 | 0 | 92 | 8 | 0 | 84 |
| 3 | 750/1 | 1.5 | >16 | 20 | 72 | 3 | 6 | 92 |
| 4 | 250/1 | 0.5 + | 10 | <1 | 62 | 37 | 0 | 26* |
| | | 0.5 rac- | | | | | | |

*Racemic product was added in this experiment therefore a high e.e. was not expected.

### C. Further Optimization of Catalyst Loading and Substrate Concentration

Further investigation into the effect of substrate concentration at catalyst loadings of S/C 500/1 and 1,000/1 was performed (Table 12). A stock solution of (R)-Phanephos and [RuCl₂(p-cym)]₂ (1.2:1 eq.) was made in DCM and appropriate volumes of the solution was added to each Endeavor vial before the DCM was blown off with N₂. The substrate (192 mg, 1 mmol) was weighed out into the Endeavor vials. Methanol (2 mL, 4 mL or 5 mL, to make desired [S]) was added to each vial followed by triethylamine (1 eq.). The vials were transferred to an Endeavor, the Endeavor was sealed and set to stir at 650 rpm, purged with nitrogen 5 times, hydrogen 5 times and heated to 50° C. at 30 bar H2. After 16 hours, the Endeavor was vented and purged with nitrogen. About 0.1 mL sample of each reaction was diluted to about 1 mL with MeOH for SFC analysis.

These experiments confirmed that, under the conditions tested, increasing the substrate concentration beyond 0.2 M decreased the e.e. values. Similar results were obtained at the two loadings tested, except for the experiment using the lowest loading and highest substrate concentration (entry 4) in which there was still a small amount of substrate remaining and the product e.e. was considerably lower than the other results.

TABLE 12
Lower Catalyst Loading Screen for (R)-Phanephos and
[RuCl₂(p-cym)]₂ and Screen for Substrate
Concentration-S/C 500/1-1,000/1, [S] = 0.2-0.5M,
MeOH, 1 eq. NEt₃, 50° C., 30 bar H₂, 16 hours

| | Catalyst | | | | | | |
|---|---|---|---|---|---|---|---|
| | Loading | [S] | S.M. | P2 | P1 | Others | e.e. |
| Entry | (S/C) | (M) | (%) | (%) | (%) | (%) | (%) |
| 1 | 500/1 | 0.5 | 0 | 93 | 7 | 0 | 87 |
| 2 | 500/1 | 0.25 | 0 | 94 | 6 | 0 | 88 |
| 3 | 500/1 | 0.2 | 0 | 95 | 5 | 0 | 89 |
| 4 | 1,000/1 | 0.5 | 4 | 87 | 9 | 1 | 82 |
| 5 | 1,000/1 | 0.25 | 0 | 94 | 6 | 0 | 88 |
| 6 | 1,000/1 | 0.2 | 0 | 94 | 6 | 0 | 89 |

### D. Screening of Shorter Reaction Time

Up until this point the reaction length was been kept at 16 hours, therefore a 3-hour reaction length was used to explore whether there is any difference on the e.e. values obtained if the reaction is stopped earlier. Different amounts of triethylamine (1 or 2 equivalents with respect to the substrate) were also tested at different substrate concentrations (Table 13). A stock solution of (R)-Phanephos and [RuCl₂(p-cym)]₂ (1.2:1 eq.) was made in DCM and appropriate volumes of the solution was added to each Endeavor vial before the DCM was blown off with N₂. The substrate (192 mg, 1 mmol) was weighed out into the Endeavor vials. Methanol (2 mL or 5 mL, to make desired [S]) was added to each vial followed by triethylamine (1 or 2 eq., 140 or 280 μL). The vials were transferred to an Endeavor, the Endeavor was sealed and set to stir at 650 rpm, purged with nitrogen 5 times, hydrogen 5 times and heated to 50° C. at 30 bar H2. After 3 hours, the Endeavor was vented and purged with nitrogen. About 0.1 mL sample of each reaction was diluted to about 1 mL with MeOH for SFC analysis.

The reactions at the higher catalyst loading, S/C 500/1, were ≥95% complete after the 3-hour reaction time, when 1 equivalent of triethylamine was used. 2 equivalents of triethylamine were shown to slow down the hydrogenation reaction compared to when 1 equivalent was used. The increased amount of triethylamine did not improve the e.e. values.

There was more evidence for improved results at lower substrate concentrations with regards to a higher e.e. and a higher conversion obtained under all conditions tested. By comparison of these results (Table 13) to the previous results in Table 12 using a 16-hour reaction time, there is a slight improvement in the e.e. values (increase up to 2%) obtained with a 3-hour reaction time. However, the reactions are not fully complete in this shorter time and so a comparison of the e.e. values at the time at which the reaction reaches completion and an extended reaction time cannot be extracted from these results.

TABLE 13
Screening of Reaction at 3 hours-S/C 500/1-1,000/1,
[S] = 0.2-0.5M, MeOH, 1-2 eq. NEt₃, 50° C., 30 bar H₂, 3 hours

| | Catalyst | | NEt₃ | | | | | |
|---|---|---|---|---|---|---|---|---|
| | Loading | [S] | no. of | S.M. | P2 | P1 | Others | e.e. |
| Entry | (S/C) | (M) | eq. | (%) | (%) | (%) | (%) | (%) |
| 1 | 500/1 | 0.5 | 1 | 5 | 89 | 6 | 1 | 88 |
| 2 | 500/1 | 0.2 | 1 | 3 | 93 | 4 | 0 | 91 |
| 3 | 500/1 | 0.5 | 2 | 45 | 51 | 4 | 0 | 87 |
| 4 | 500/1 | 0.2 | 2 | 26 | 70 | 4 | 0 | 89 |
| 5 | 1,000/1 | 0.5 | 1 | 31 | 65 | 4 | 0 | 89 |
| 6 | 1,000/1 | 0.2 | 1 | 9 | 87 | 4 | 0 | 91 |
| 7 | 1,000/1 | 0.5 | 2 | 41 | 55 | 4 | 0 | 86 |
| 8 | 1,000/1 | 0.2 | 2 | 26 | 71 | 3 | 0 | 91 |

### E. Screening of Temperature and NEts Amount

Lower triethylamine equivalents (0.5 eq) using a catalyst loading of S/C 1000/1 was tested at two substrate concentrations and at three temperature settings (Table 14). A stock solution of (R)-Phanephos and [RuCl₂(p-cym)]₂ (1.2:1 eq.) was made in DCM and appropriate volumes of the solution was added to those vials before the DCM was blown off with N₂. Substrate (192 mg, 1 mmol) was weighed out into Endeavor vials. Methanol (2 or 5 mL for 0.5 or 0.2 M substrate concentration respectively) was added into each vial followed by triethylamine (1 or 0.5 eq., 140 or 70 μL). The vials were transferred to an Endeavor, the Endeavor was sealed and set to stir at 650 rpm, purged with nitrogen 5 times, hydrogen 5 times and heated to 40-60° C. at 30 bar H2. After 16 hours, the Endeavor was vented and purged with nitrogen. About 0.1 mL sample of each reaction was diluted to about 1 mL with MeOH for SFC analysis.

Using 0.5 eq. of NEts instead of 1 for the conditions tested at 50° C. showed that for both substrate concentrations an improvement in the e.e., as well as slight improvement on conversion for the higher substrate concentration, was obtained (Table 14, entries 3-6). The effect of temperature is less obvious, however the best e.e. values for each substrate concentration are obtained at 40° C. (entries 1-2).

TABLE 14
Temperature and NEt₃ Equivalent Screen-S/C 1,000/1,
[S] = 0.2-0.5M, MeOH 0.5-1 eq. NEt₃,
40-60° C., 30 bar H₂, 16 hours

| | | NEt₃ | | | | | | |
|---|---|---|---|---|---|---|---|---|
| | [S] | no. of | Temp. | S.M. | P2 | P1 | Others | e.e. |
| Entry | (M) | eq. | (° C.) | (%) | (%) | (%) | (%) | (%) |
| 1 | 0.2 | 1 | 40 | 0 | 96 | 4 | 0 | 93 |
| 2 | 0.5 | 1 | 40 | 7 | 87 | 6 | 1 | 88 |
| 3 | 0.2 | 1 | 50 | 0 | 94 | 6 | 0 | 89 |
| 4 | 0.5 | 1 | 50 | 4 | 87 | 9 | 1 | 82 |
| 5 | 0.2 | 0.5 | 50 | 0 | 95 | 5 | 0 | 90 |
| 6 | 0.5 | 0.5 | 50 | <1 | 93 | 7 | 0 | 86 |
| 7 | 0.2 | 1 | 60 | 0 | 94 | 6 | 0 | 88 |
| 8 | 0.5 | 1 | 60 | 0 | 91 | 9 | 0 | 82 |

### F. Screening of Pressure for Hydrogenation

Up to this point, 30 bar has been maintained as the pressure used. Thus, the effect of using lower pressure on the results was investigated (Table 15). A stock solution of (R)-Phanephos and [RuCl₂(p-cym)]₂ (1.2:1 eq.) was made in DCM and appropriate volumes of the solution was added to those vials before the DCM was blown off with N₂. Substrate (192 mg, 1 mmol) was weighed out into Endeavor vials. Methanol (2 or 5 mL for 0.5 or 0.2 M substrate concentration respectively) was added into each vial followed by triethylamine (0.5 eq., 70 μL). The vials were transferred to an Endeavor, the Endeavor was sealed and set to stir at 650 rpm, purged with nitrogen 5 times, hydrogen 5 times and heated to 40-50° C. at 5-30 bar H2. After 16 hours, the Endeavor was vented and purged with nitrogen. About 0.1 mL sample of each reaction was diluted to about 1 mL with MeOH for SFC analysis. The hydrogen uptake time is approximated from the data recorded by the Endeavor which shows at what time the uptake has stopped, therefore the reaction is assumed to be >90% complete at this point. No data for H2 uptake time for entries 1-2 were obtained because the Endeavor hydrogen uptake curves indicated there were leaks.

Very encouragingly the pressure could be decreased to 5 bar and full conversion was still obtained at S/C 1,000/1. The high e.e. was also maintained at this pressure and loading (Table 15, entry 6). Decreasing the pressure was seen to cause a decreased reaction rate, for example requiring 7 hours instead of 3 h to reach full conversion with S/C 1,000/1 at 5 bar instead of 10 bar (compare entries 3 and 6). Using higher catalyst loading decreased the required reaction time (compare entries 6-8).

TABLE 15
Screening for Different Pressure Conditions-S/C 200/1-1,000/1,
[S] = 0.2-0.5M, MeOH, 0.5 eq. NEt₃, 40-50° C., 5-30 bar H₂, 16 hours

| | | | | | H₂ | | | | | |
|---|---|---|---|---|---|---|---|---|---|---|
| | | Cat. | | | Uptake | | | | | |
| | Pressure | Loading | [S] | Temp. | Time | S.M. | P2 | P1 | Others | e.e. |
| Entry | (bar) | (S/C) | (M) | (° C.) | (h) | (%) | (%) | (%) | (%) | (%) |
| 1 | 30 | 1,000/1 | 0.5 | 40 | n.d. | 0 | 93 | 7 | 0 | 86 |
| 2 | 30 | 1,000/1 | 0.2 | 40 | n.d. | 0 | 95 | 5 | 0 | 91 |
| 3 | 10 | 1,000/1 | 0.2 | 50 | 3 | 0 | 95 | 5 | 0 | 91 |
| 4 | 10 | 500/1 | 0.2 | 50 | 2 | 0 | 96 | 5 | 0 | 91 |
| 5 | 10 | 200/1 | 0.2 | 50 | 1 | 0 | 96 | 4 | 0 | 91 |
| 6 | 5 | 1,000/1 | 0.2 | 50 | 7 | 0 | 96 | 4 | 0 | 92 |
| 7 | 5 | 500/1 | 0.2 | 50 | 5 | 0 | 96 | 4 | 0 | 92 |
| 8 | 5 | 200/1 | 0.2 | 50 | 2 | 0 | 96 | 4 | 0 | 92 |

### G. Design of Experiments (DoE)

Up to now, the results showed that reactions were successful at 5 bar and with a catalyst loading of S/C 1,000/1. These conditions were used to further explore the effects of factors: substrate concentration, amount of triethylamine and temperature. A Design of Experiments (DoE) approach was used in order to extract the trends caused by each of these factors and attempt to find conditions which optimize the conversion and selectivity. The experiments generated by the DoE model were carried out on a 1 mmol substrate scale. The experimental results are shown in Table 16. A stock solution of (R)-Phanephos and [RuCl₂(p-cym)]₂ (1.2:1 eq.) was made in DCM and appropriate volumes of the solution was added to those vials before the DCM was blown off with N2. Substrate (192 mg, 1 mmol) was weighed out into Endeavor vials. Methanol (1, 1.7 or 5 mL for 1.0, 0.6 or 0.2 M substrate concentration respectively) was added into each vial followed by triethylamine (42, 91 or 140 μL for 0.3, 0.65 or 1 eq. respectively). The vials were transferred to an Endeavor, the Endeavor was sealed and set to stir at 650 rpm, purged with nitrogen 5 times, hydrogen 5 times and heated to 40-50° C. at 5 bar H2. After 16 hours, the Endeavor was vented and purged with nitrogen. About 0.1 mL sample of each reaction was diluted to about 1 mL with MeOH for SFC analysis. The hydrogen uptake time is approximated from the data recorded by the Endeavor which shows at what time the uptake has stopped, therefore the reaction is assumed to be ≥90% complete at this point. No data for H2 uptake time for entry 3 was obtained because of a leak.

TABLE 16
DoE Investigation of Variables-S/C 1,000/1, [S] = 0.2-1.0M,
MeOH, 0.3-1.0 eq. NEt₃, 40-50° C., 5 bar H₂, 16 hours

| | | NEt₃ | | H₂ | | | | | |
|---|---|---|---|---|---|---|---|---|---|
| | | no. | | Uptake | | | | | |
| | [S] | of | Temp. | Time | S.M. | P2 | P1 | Others | e.e. |
| Entry | (M) | eq. | (° C.) | (h) | (%) | (%) | (%) | (%) | (%) |
| 1 | 0.2 | 1.0 | 40 | 8 | <1 | 96 | 3 | 0 | 94 |
| 2 | 0.2 | 0.3 | 50 | 6 | 0 | 95 | 4 | 0 | 92 |
| 3 | 0.2 | 0.3 | 40 | n.d. | 0 | 96 | 3 | 0 | 93 |
| 4 | 1.0 | 0.3 | 50 | 8 | 1 | 86 | 11 | 2 | 77 |
| 5 | 1.0 | 0.3 | 40 | >16 | 9 | 84 | 6 | 2 | 87 |
| 6 | 1.0 | 1.0 | 40 | >16 | 37 | 60 | 3 | 0 | 89 |
| 7 | 0.6 | 0.65 | 45 | 10 | 1 | 93 | 6 | 1 | 88 |
| 8 | 0.2 | 1.0 | 50 | 10 | 1 | 92 | 7 | 1 | 86 |
| 9 | 1.0 | 1.0 | 40 | >16 | 29 | 68 | 4 | 0 | 90 |
| 10 | 0.2 | 1.0 | 50 | 7 | 0 | 95 | 5 | 0 | 91 |
| 11 | 0.6 | 0.65 | 45 | 15 | 0 | 93 | 6 | 1 | 88 |
| 12 | 1.0 | 1.0 | 50 | >16 | 21 | 71 | 2 | 6 | 96* |
| 13 | 1.0 | 0.3 | 40 | >16 | 30 | 63 | 5 | 2 | 86 |
| 14 | 1.0 | 0.3 | 50 | 15 | 1 | 90 | 8 | 1 | 83 |
| 15 | 0.2 | 0.3 | 50 | 8 | 0 | 94 | 5 | 1 | 90 |
| 16 | 0.2 | 1.0 | 40 | 15 | 1 | 95 | 4 | 1 | 93 |

*The true e.e. value is likely to be lower because there is some methyl ester impurity overlapping with the peak for P2.

The results (Table 16) were entered into the DoE software, JMP. The model shows that substrate concentration has the largest effect out of the factors (as seen in the effect summary table by the very low PValue) with the other factors having a significantly lower effect on results (Table 17). The prediction profiler, predicted that as the substrate concentration is increased across the 0.2 to 1.0 M range, the “desirability” (i.e. maximizing conversion and e.e. simultaneously) has a steep decline. By the prediction profiler model, the amount of triethylamine and temperature have much less of an effect on the desirability.

The DoE software predicted that the best results will be obtained at the lowest concentration with the lowest amount of triethylamine and lowest temperature from the ranges tested: 0.2 M, 0.3 eq. of NEt₃ and 40° C. This is reflected by the best result obtained experimentally: >99% conversion and 93% e.e. (Table 16, entry 3).

TABLE 17
DoE Prediction Profile-Effect Summary of Variables

| Source | LogWorth | PValue |
|---|---|---|
| [S](0.2, 1) | 3.045 | 0.00090 |
| [S]*eq. of NEt₃ | 1.766 | 0.01714 |
| eq. of NEt₃(0.3, 1) | 1.217 | 0.06062 \hat{ } |
| Temp.(40, 50) | 0.892 | 0.12810 |
| [S]*Temp. | 0.852 | 0.14049 |
| eq. of NEt3*Temp | 0.685 | 0.20672 |

(‘\hat{ }’ denotes effects with | containing effects above them)

The prediction profiler can also be used to calculate which conditions will give the best results at a desired substrate concentration. These generated results are shown in Table 18. These results suggest that it is unlikely to be able to achieve a conversion >99% and high e.e. using a concentration greater than 0.2 M with these sets of conditions. However, it must be noted that it can be seen from the hydrogen uptakes that the reactions at higher concentration are slower and thus have not reached completion within the 16-hour timeframe tested in these

TABLE 18
DoE Optimization Results for Different Substrate Concentration

| | No of eq. | Temp | Conversion | e.e. | |
|---|---|---|---|---|---|
| [S] | of Net₃ | (° C.) | (%) | (%) | Desirability* |
| 1.0 | 0.5 | 50 | 90.8 | 85.1 | 0.4 |
| 0.5 | 0.3 | 43 | 95.1 | 89.9 | 0.6 |
| 0.4 | 0.3 | 40 | 95.0 | 93.0 | 0.7 |
| 0.3 | 0.3 | 40 | 97.2 | 94.4 | 0.8 |
| 0.2 | 0.3 | 40 | 99.5 | 95.8 | 0.9 |

*Desirability values are between 0 and 1. The desirability is set to maximize both conversion and e.e. value with equal importance and with high, middle and low values set at 100, 90 and 80 for both responses.

### H. Screening for Reaction Time

The results from the DoE study found that when using conditions within the ranges explored (S/C 1,000/1, [S]=0.2-1.0 M, MeOH, 0.3-1.0 eq. NEt₃, 40-50° C., 5 bar H2, 16 hours) it would not be possible to obtain simultaneous high conversion (>95%) and enantioselectivity (>90%) at substrate concentrations greater than 0.5 M. It was therefore tested whether a longer reaction time would allow for greater conversion at 0.6-1.0 M substrate concentration (Table 19). A stock solution of (R)-Phanephos and [RuCl₂(p-cym)]₂ (1.2:1 eq.) was made in DCM and appropriate volumes of the solution was added to those vials before the DCM was blown off with N₂. Substrate (192 mg, 1 mmol) was weighed out into Endeavor vials. Methanol (1, 1.3 or 1.7 mL for 1.0, 0.8 or 0.6 M substrate concentration respectively) was added into each vial followed by triethylamine (91, 112 or 140 μL for 0.65, 0.8 or 1 eq. respectively). The vials were transferred to an Endeavor, the Endeavor was sealed and set to stir at 650 rpm, purged with nitrogen 5 times, hydrogen 5 times and heated to 45-50° C. at 5 bar H2. After 16 or 24 hours, the Endeavor as vented and purged with nitrogen. About 0.1 mL sample of each reaction was diluted to about 1 mL with MeOH for SFC analysis. No data for H2 uptake time for entry 1 was obtained because of a leak.

The reactions using 0.8 M or 1.0 M substrate concentration were not complete within 24 hours (entries 1-2).

TABLE 19
Reactions Stopped After 24 Hours-S/C 1,000/1, [S] = 0.6-1.0M,
MeOH, 0.65-1.0 eq. NEt₃, 45-50° C., 5 bar H₂, 24 hours

| | | NEt₃ | | H₂ | | | | | |
|---|---|---|---|---|---|---|---|---|---|
| | | no. | | Uptake | | | | | |
| | [S] | of | Temp. | Time | S.M. | P2 | P1 | Others | e.e. |
| Entry | (M) | eq. | (° C.) | (h) | (%) | (%) | (%) | (%) | (%) |
| 1 | 1.0 | 1.0 | 50 | n.d. | 15 | 81 | 4 | 0 | 90 |
| 2 | 0.8 | 1.0 | 50 | >24 | 5 | 89 | 5 | 1 | 89 |
| 3 | 0.6 | 1.0 | 50 | >24 | 1 | 92 | 7 | 1 | 87 |
| 4 | 0.6 | 0.8 | 50 | 10 | 0 | 93 | 7 | 0 | 86 |

### I. Screening for Types and Amounts of Base

A couple of other bases were tested to see if they would provide any benefit (Table 20). Same procedure was followed for the temperature screen (section H), except for the addition of triethylamine or base was adjusted as shown in Table 20, and the reaction was stopped at 16 hours. No data for H2 uptake time for entries 1 and 5 were obtained because of a leak.

NaOMe and Na₂CO₃ both gave similar results to NEt₃, when using 0.3 equivalents of base to substrate (entries 1-3, 5). Using 0.6 equivalents of NaOMe or Na₂CO₃ gave slightly lower conversions than when 0.3 equivalents were used (entries 3-6). Therefore, there was no advantage seen for using NaOMe/Na₂CO₃ instead of NEt₃. Two different substrate batches were tested under the same conditions and found to give similar results (entries 1-2). The substrate batches had similar purity as determined by 1H NMR (96%, 95% for 1st and 2nd batch). It must be noted however that SFC analysis of substrate batch 2 shows the appearance of a late-eluting peak (8.6 minutes) with <1% integration, which was not seen in the first batch. The 1% “others” for reactions using this substrate batch thus mainly relates to the presence of this peak on the SFC chromatogram.

TABLE 20
Screening for Base-S/C 1,000/1, [S] = 0.4M, MeOH,
0.3-0.6 eq. base, 40° C., 5 bar H₂, 16 hours

| | | | No. | | | | | | |
|---|---|---|---|---|---|---|---|---|---|
| | | | of | H₂ | | | | | |
| | | | Eq. | Uptake | | | | | |
| | S.M. | | of | Time | S.M. | P2 | P1 | Others | e.e. |
| Entry | Batch | Base | Base | (h) | (%) | (%) | (%) | (%) | (%) |
| 1 | 1 | Net₃ | 0.3 | n.d. | 0 | 96 | 3 | 0 | 93 |
| 2 | 2 | Net₃ | 0.3 | 9 | 0 | 96 | 4 | 1 | 92 |
| 3 | 2 | NaOMe | 0.3 | 14 | 0 | 95 | 4 | 1 | 91 |
| 4 | 2 | NaOMe | 0.6 | 16 | <1 | 95 | 4 | 1 | 91 |
| 5 | 2 | Na₂CO₃ | 0.3 | n.d. | 0 | 95 | 5 | 0 | 91 |
| 6 | 2 | Na₂CO₃ | 0.6 | 7 | 2 | 94 | 4 | 1 | 92 |

Because the previous reactions were successful with 0.4 M substrate concentration, additional conditions were tested using 0.6 M. This included testing lower amounts of NaOMe and Na₂CO₃ as well as testing different Ru precursors (Table 21). A=[RuCl₂(p-cym)]₂, B=Ru(COD)(Me-allyl)₂, C=Ru(COD)(TFA)₂. No data for H₂ uptake time for entry 7 was obtained because of a leak.

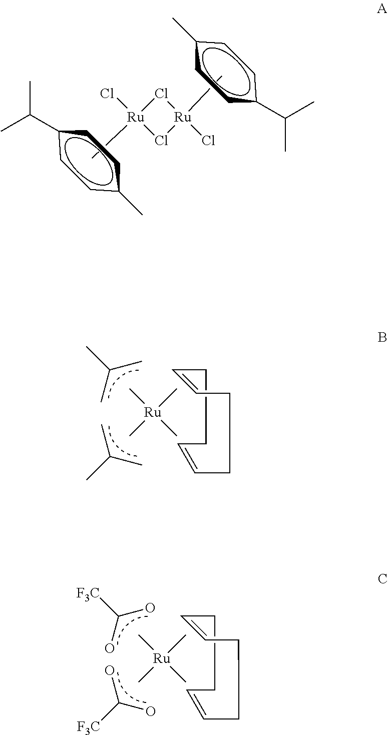

The reactions were found to be successful (i.e. complete conversion and ≥90% e.e.) at this higher substrate concentration of 0.6 M. It therefore shows the requirement to obtain these results is to use lower amounts of base (0.1-0.3 eq.) and lower temperature (40° C.). The alternative bases, NaOMe and Na₂CO₃, were again showed to give similar results to NEt₃ and the amounts could be decreased to 0.1 equivalent (entries 1-6).

The different Ru precursors, B and C, gave very similar results to [RuCl₂(p-cym)]₂ (A) with an e.e. difference of +1%. Thus, this is reassurance that it is not the Cl ligands present in the active complex which are influencing the maximum e.e. able to be obtained for this reaction.

TABLE 21
Base and Catalyst Precursor Screen at 0.6M Substrate-S/C 1,000/1,
[S] = 0.6M, MeOH, 0.1-0.3 eq. base, 40° C., 5 bar H₂, 16 hours

| | | | | H₂ | | | | | |
|---|---|---|---|---|---|---|---|---|---|
| | | No. of | | Uptake | | | | | |
| | | Eq. of | Ru | Time | S.M. | P2 | P1 | Others | e.e. |
| Entry | Base | Base | precursor | (h) | (%) | (%) | (%) | (%) | (%) |
| 1 | Net₃ | 0.3 | A | 7 | 0 | 94 | 5 | 1 | 90 |
| 2 | Net₃ | 0.1 | A | 7 | 0 | 94 | 5 | 1 | 90 |
| 3 | NaOMe | 0.3 | A | 7 | 0 | 93 | 5 | 2 | 89 |
| 4 | NaOMe | 0.1 | A | 7 | 0 | 94 | 5 | 1 | 91 |
| 5 | Na₂CO₃ | 0.3 | A | 8 | 0 | 94 | 5 | 1 | 91 |
| 6 | Na₂CO₃ | 0.1 | A | 8 | 0 | 94 | 5 | 1 | 90 |
| 7 | Net₃ | 0.3 | B | n.d. | 0 | 95 | 5 | 1 | 91 |
| 8 | Net₃ | 0.1 | A | 6 | 0 | 96 | 3 | 0 | 93 |
| 9 | Net₃ | 0.1 | B | 7 | 0 | 96 | 45 | 1 | 92 |
| 10 | Net₃ | 0.1 | C | 6 | 0 | 96 | 3 | 1 | 94 |

### J. Reaction Screening in Parr Vessels (25 mL)

From the previous results, 0.6 M was found to give full conversion with a 90-93% e.e. value. These conditions were used for a scale-up into a 25 mL Parr vessel using 1.6 g of substrate and 14 mL MeOH (Table 22). (R)-Phanephos and [RuCl₂(p-cym)]₂ (1.2:1 eq., 5.8 mg, 2.6 mg respectively) were weighed into a 25 mL Parr vessel followed by the substrate (1.614 g, 8.4 mmol). Methanol (14 mL, 0.6 M substrate concentration) was added to the vessel followed by triethylamine (118 μL, 0.84 mmol, 0.1 eq.). The vessel was sealed and purged with nitrogen 5 times (at ˜2 bar) and 5 times with stirring (˜500 rpm). The vessel was then purged with hydrogen 5 times (at ˜10 bar) and 5 times with stirring (˜500 rpm). The vessel was then pressurized to 5 bar hydrogen pressure and heated to 40° C. (with stirring set as 500 rpm). The pressure was kept constant but with venting and refilling to 5 bar after sampling. Reaction was sampled at 0.5, 1.5, 2.5, 3.5, 4.5, 5.5, and 70 hours. After 70 hours, the vessel was allowed to cool, vented and purged with nitrogen. Each ˜0.1 mL sample was diluted to ˜1 mL with MeOH used for SFC analysis.

Comparing the rate of reaction for the reaction carried out in the Parr vessel with the reaction in the Endeavor showed a slower reaction for the larger scale reaction (FIG. 4). This difference could arise from the difference in the mixing efficiency of the Endeavor vs. Parr. The reaction was performed using a low stirring speed (500 rpm) and with an extended reaction time in order to test for robustness of the catalyst system and the process on scale-up. This showed a slower rate and a lower e.e. value than was obtained in the Endeavor. There is scope to increase the stirring speed in the Parr vessel.

No reaction sampling was done between 5.5-70 hours thus it is unknown whether there was e.e. degradation from heating beyond the time at which full conversion is reached. By extrapolating the rate curve beyond the first 6 hours, it appears that the reaction would have been likely to have been complete in about 15-20 hours.

TABLE 22
Hydrogenation in Parr Vessel-S/C 1,000/1, [S] = 0.6M, 114 g/L,
MeOH, 0.1 eq. of NEt3, 40° C., 5 bar H₂, 70 hours, 500 rpm

| | Time | S.M. | P2 | P1 | Others | e.e. |
|---|---|---|---|---|---|---|
| Entry | (h) | (%) | (%) | (%) | (%) | (%) |
| 1 | 0.5* | 97 | 3 | 0 | 0 | — |
| 2 | 1.5 | 92 | 6 | 1 | 1 | — |
| 3 | 2.5 | 84 | 14 | 1 | 1 | 82 |
| 4 | 3.5 | 76 | 22 | 2 | 0 | 86 |
| 5 | 4.5 | 69 | 29 | 2 | 0 | 85 |
| 6 | 5.5 | 60 | 37 | 3 | 0 | 84 |
| 7 | 70.0 | 0 | 93 | 7 | 1 | 87 |

*This sample was taken at the point at which the internal temperature of vessel had reached 40° C.

Next, the speed of the stirring in the Parr was increased to the maximum speed (>1500 rpm) in order to see whether this would achieve more similar results to the Endeavor (Table 23). This Parr reaction, using maximum stirring speed, shows a faster rate compared with the slower stirring speed reaction, with the reaction appearing to be complete (as assessed by hydrogen uptake) at around 10 hours instead of approximately 18 hours (500 rpm).

The higher stirring speed did not make all the difference to the results between Parr and Endeavor as the Endeavor reaction was complete faster, in about 7 hours. Notably, the enantioselectivity did not been improve by the increased stirring speed. The same result of 87% e.e. has been obtained at the end of the reaction for both Parr reactions (Tables 22 and 23), compared to the 90-93% e.e. obtained using the same set of conditions in the Endeavor.

TABLE 23
Hydrogenation in 25 mL Parr Vessel (1.6 g S.M.)-S/C
1,000/1, [S] = 0.6M, 114 g/L, MeOH (14 mL), 0.1
eq. of NEt3, 40° C., 5 bar H₂, 20.5 hours, >1500 rpm

| | Time | S.M. | P2 | P1 | Others | e.e. |
|---|---|---|---|---|---|---|
| Entry | (h) | (%) | (%) | (%) | (%) | (%) |
| 1 | 1.0 | 90 | 9 | 1 | 0 | 82 |
| 2 | 2.0 | 83 | 15 | 2 | 1 | 82 |
| 3 | 17.5 | 0 | 92 | 7 | 1 | 86 |
| 4 | 20.5 | 0 | 93 | 7 | 1 | 87 |
| 5 | After | 0 | 92 | 7 | 1 | 85 |
| | work-up* | | | | | |

*Work-up procedure: MeOH removed by concentrating under vacuum, followed by addition of EtOAc (10 mL) and 1M HCl (10 mL). The layers were mixed before separating. The EtOAc layer was washed with a further portion of 1M HCl (4 mL) before removing the aqueous layer to leave the EtOAc organic phase. The aqueous layer was then washed with a further portion of EtOAc (4 mL) and the organic layers were combined. EtOAc was then removed under vacuum to leave behind the product as a greyish solid.

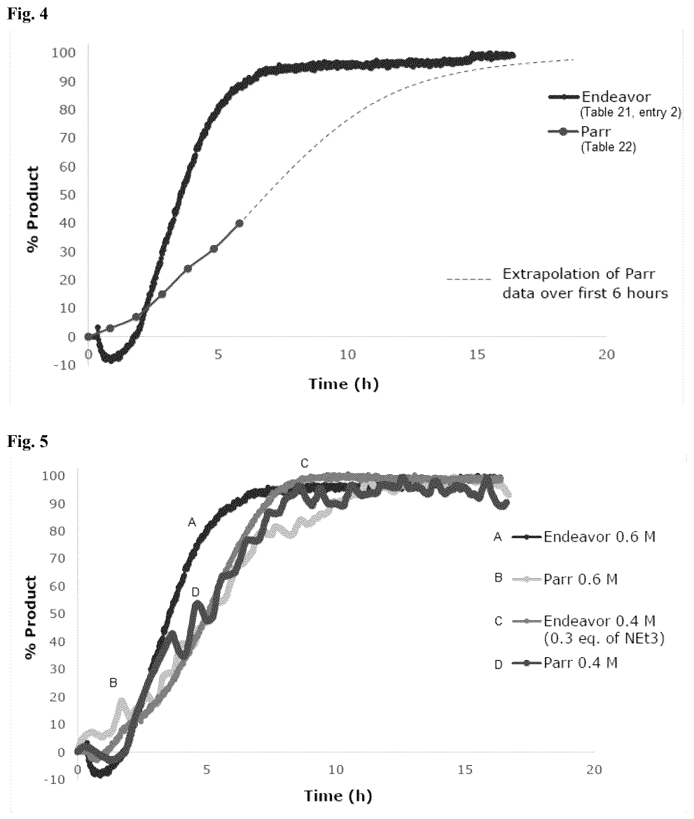

The reaction set-up shown in Table 23 was repeated in the 25 mL Parr with a lower substrate concentration, to probe whether this could achieve greater enantioselectivity as was seen during the small-scale screening of substrate concentrations (in the Endeavor). This reaction was carried out at 0.4 M and sampling was only carried out at the end of the reaction; however, the hydrogen uptake can be used to give information on the rate of reaction (Table 24, FIG. 5).

TABLE 24
Hydrogenation in 25 mL Parr Vessel (1.1 g S.M.)-S/C
1,000/1, [S] = 0.4M, 77 g/L, MeOH (14 mL),
0.1 eq. of NEt3, 40° C., 5 bar H₂, 20.5 hours, >1500 rpm

| | Time | S.M. | P2 | P1 | Others | e.e. |
|---|---|---|---|---|---|---|
| Entry | (h) | (%) | (%) | (%) | (%) | (%) |
| 1 | 17 | 0 | 93 | 7 | 1 | 87 |
| 2 | 20 | 0 | 93 | 6 | 1 | 87 |
| 3 | After work-up* | 0 | 92 | 7 | 1 | 86 |

*Same work-up procedure as Table 23.

The results showed that higher enantioselectivity was not obtained by this decrease in substrate concentration, with 87% e.e. obtained at both concentrations. From the hydrogen uptakes recorded, the lower concentration reaction appears to have a faster initial rate and reach completion in a shorter time, ˜9 hours, compared to the higher concentration reaction which appears complete in ˜11 hours (FIG. 5). This is more similar to the reaction times of the reactions carried out in the Endeavor (with 0.3 eq. NEt₃). In the Endeavor, however, reaction using 0.1 equivalent of triethylamine at 0.4 M has not been carried out (higher amounts of triethylamine is known to slow down the reaction).

A difference between the procedures used to set up reactions in the Endeavor and the Parr vessel is that for the Endeavor reactions, due to the small scale, a stock solution of metal precursor and ligand was made up in DCM and small volumes were added to vials to give the correct catalyst loading (before the DCM was evaporated), whereas in the Parr the precursor and ligand were both weighed directly into the vessel as solids. Thus, the Parr reactions can be described as undergoing ‘in situ’ formation of the metal-ligand complex with the substrate present, whereas for the Endeavor reactions the metal and ligand would have pre-complexed before the substrate was added. Therefore, to investigate the difference this was causing, procedure variations were tested in the Endeavor (Table 25). All masses of [RuCl₂(p-cym)]₂ and (R)-Phanephos were weighed out to give S/C 1,000/1 and a 1.2 molar eq. of the ligand. For the ‘in situ’ procedure a stock solution of [RuCl₂(pcym)]₂ in DCM was added to one side of an Endeavor vial before the DCM was blown off with N₂ and a stock solution of (R)-Phanephos in DCM was added to the opposite side of the vial before DCM was removed (thus the metal and ligand do not have contact before the other reagents are added). For the pre-mix procedure a stock solution of (R)-Phanephos and [RuCl₂(p-cym)]₂ (1.2:1 eq.) was made in DCM or MeOH and appropriate volumes of the solution was added to the vials before the solvent was blown off with N₂. Substrate (192 mg, 1 mmol) was weighed out into the Endeavor vials. Methanol (1.7 mL, 0.6 M substrate concentration) was added into each vial followed by triethylamine (14 μL, 0.1 eq.). The vials were transferred to an Endeavor, the Endeavor was sealed and set to stir at 650 rpm, purged with nitrogen 5 times, hydrogen 5 times and heated to 40° C. at 5 bar H2. After 16 hours, the Endeavor was purged with nitrogen. A ˜0.1 mL sample of each reaction was diluted to ˜1 mL with MeOH for SFC analysis

The results were all very similar with 91-92% e.e. obtained in all cases. This suggests that the lower e.e. obtained in the Parr vessel is not due to the absence of a pre-mix of metal precursor and ligand. This leaves the following as potential causes for lower e.e. values: contamination in the Parr vessel leading to a racemic background reaction, hydrogen starvation due to a less than optimal headspace in the reactor, difference in accuracy of internal temperature meaning that the Endeavor reactions were actually at less than 40° C.

Significantly, the ‘in situ’ reactions which were vented at 10 or 16 hours gave the same result thus there is no e.e. degradation over this 6-hour period after the reaction has been complete.

TABLE 25
Comparison of different procedures for the addition of
metal precursor and ligand-S/C 1,000/1, [S] = 0.6M,
MeOH, 0.1 eq. NEt3, 40° C., 5 bar H₂, 16 hours

| | Catalyst | Time | S.M. | P2 | P1 | Others | e.e. |
|---|---|---|---|---|---|---|---|
| Entry | Procedure | (h) | (%) | (%) | (%) | (%) | (%) |
| 1 | ‘In situ’- | 10* | 0 | 95 | 4 | 1 | 92 |
| | Ru + Ligand | | | | | | |
| 2 | ‘In situ’- | 16 | 0 | 95 | 4 | 1 | 92 |
| | Ru + Ligand | | | | | | |
| 3 | Pre-mix Ru + | 16 | 0 | 95 | 4 | 0 | 91 |
| | Ligand in DCM | | | | | | |
| 4 | Pre-mix Ru + | 16 | 0 | 95 | 4 | 1 | 91 |
| | Ligand in MeOH | | | | | | |

*This vessel was set to vent after 10 hours and stop heating (measured temperature was 30° C. from 10-16 hours).

### K. Investigation of Background Reactions

Three runs (testing two stirring speeds and two substrate concentrations) using a 25 mL Parr vessel, at S/C 1,000/1, have been found to give lower results than expected based on the Endeavor results. Thus, it was tested whether there was a background reaction present in the vessel which was causing the lower enantioselectivity. The conditions were therefore kept the same apart from no addition of ligand or metal precursor and the pressure was kept constant but with venting and refilling to the desired pressure after sampling. After 5 hours at 20 bar, the pressure was decreased to 5 bar (Table 26).

By initially using 20 bar as the hydrogen pressure, there was 11% of low e.e. product measured from sampling after 5 hours (Table 26, entry 2). After 5 hours the pressure was decreased to 5 bar. After a further 15.5 hours of heating and maintaining 5 bar pressure, there was a further 3% of product made (Table 26, entry 3).

The rate of the background reaction is thus lower at lower pressure and will have less of an impact on the e.e. obtained in a reaction (Table 27). This experiment is evidence for the presence of a background reaction and explains the lower e.e. obtained in the previous experiments using this specific Parr vessel.

TABLE 26
Test of background reaction in 25 mL Parr vessel-[S] = 0.6M, MeOH,
0.1 eq. of NEt₃, 40° C., 5-20 bar H₂, >1500 rpm, 23 hours

| | Pressure | Time | S.M. | P2 | P1 | Others | e.e. |
|---|---|---|---|---|---|---|---|
| Entry | (bar) | (h) | (%) | (%) | (%) | (%) | (%) |
| 1 | 20 | 3.5 | 90 | 8 | 3 | 0 | 43 |
| 2 | 20 | 5 | 89 | 8 | 3 | 0 | 44 |
| 3 | 5 (from | 20.5 | 86 | 9 | 5 | 0 | 28 |
| | 5-20.5 h) | | | | | | |
| 4 | 5 | 23 | 85 | 10 | 5 | 1 | 35 |

TABLE 27
Analysis of background reaction rates for
specific Parr vessel and impact on e.e.

| | | | | | | | e.e. |
|---|---|---|---|---|---|---|---|
| | | | | | | | predicted |
| | | | | | | | for |
| | | | | | | | reaction |
| | | | | | Rate | | due to |
| | Cat. | Pressure | Time | Prod | Prod %/ | | racemic* |
| Entry | (S/C) | (bar) | (h) | % | hour | e.e. | background^{a} |
| 1 | 1,000/1 | 5 | 10 | 100 | 10 | 87 | — |
| 2 | none | 5 | 15.5 | 3 | 0.2 | low | 90 |
| 3 | none | 20 | 5 | 11 | 2.2 | low | 72 |

^{a}Calculated from the rate of product from background reaction under either 5 or 20 bar conditions and using 10 hours as the reaction completion time and 93% e.e. as the maximum e.e. of the enantioselective hydrogenation product.
*In this case the background reaction has been found to give a low level of enantioselectivity for the desired product enantiomer (P2).

To verify the background reaction arises from the vessel and not from a contaminant in the substrate, further background reaction studies were carried out in the Endeavor—where the previously ≥91% e.e. results had been obtained. A study had already been performed to check if there was any background reaction earlier in this project (Example 1), however at that stage 0.2 M was used as the concentration and with a different substrate batch. Thus, the two different substrate batches were tested in parallel and the conditions now found to be optimal for the enantioselective hydrogenation reaction were tested with no catalyst present (Table 28). Same reaction setup as for Table 25 except as noted in Table 28.

Both substrate batches, and a few different conditions, were found to give <1% of product, at 50° C. (entries 2-5). This indicates that the background reaction observed in the Parr vessel is likely to be due to a contaminant found in the vessel rather than in the substrate. The vials containing substrate, triethylamine and methanol were re-subjected to the Endeavor but with an increased temperature of 90° C. In this case, there was a small amount of product seen after 16 hours (entries 6-8). This is likely to be from a trace of a contaminant in the Endeavor which required these harsher conditions to react with the substrate.

TABLE 28
Background reaction in the Endeavor-[S] = 0.2-0.6M, MeOH,
0.1 eq. NEt₃, 50-90° C., 5-30 bar H₂, 250 rpm, 16 hours

| | | | | | | | | |
|---|---|---|---|---|---|---|---|---|
| | | | Gas Type | | | | | |
| | S.M. | Temp | and Pressure | S.M. | P2 | P1 | Others | e.e. |
| Entry | Batch | (C.) | (bar) | (%) | (%) | (%) | (%) | (%) |

Previous test, using 0.2M substrate conc. and no Net₃:

| | | | | | | | | |
|---|---|---|---|---|---|---|---|---|
| 1 | 1 | 90 | H₂, 30 | 100 | 0 | 0 | 0 | — |

Using 0.6M substrate conc. and 0.1 eq. Net₃:

| | | | | | | | | |
|---|---|---|---|---|---|---|---|---|
| 2 | 1 | 50 | H₂, 30 | 100 | 0 | 0 | 0 | — |
| 3 | 2 | 50 | H₂, 30 | >99 | 0 | 0 | <1 | — |
| 4 | 2 | 50 | H₂, 5 | 99 | 0 | <1 | <1 | — |
| 5 | 2 | 50 | N₂, 5 | >99 | 0 | 0 | <1 | — |
| 6 | 1 | 90 | H₂, 30 | 92 | 5 | 2 | <1 | low |
| 7 | 2 | 90 | H₂, 30 | 93 | 5 | 1 | <1 | low |
| 8 | 2 | 90 | H₂, 5 | 91 | 6 | 2 | 1 | low |
| 9 | 2 | 90 | N₂, 5 | >99 | 0 | 0 | <1 | — |

To demonstrate that in the absence of a background reaction similar results to the Endeavor could be obtained at larger scale in a Parr vessel, a glass liner was used with a PTFE stirrer bar and PTFE tape covering the thermocouple (Table 29). The reaction setup was otherwise same as Table 22, but with a substrate amount of (1.845 g, 9.6 mmol) and different reaction time as noted. For entry 1, there was an error with the hotplate used for heating this reaction overnight where the temperature fell from 40 to 22° C., but at 16 hours the reaction was heated to 40° C. again

91% e.e. was obtained at full conversion using this set-up thus showing that a contaminant in the previously used stainless steel vessel was causing the lower e.e. and thus in the absence of any background reaction, high e.e. can be obtained at the catalyst loading of S/C 1,000/1. 1H NMR spectra of the reaction product after the methanol has been removed and after the work-up has been performed showed the work-up to be successful at removing all the triethylamine. There was a 1% loss of e.e. measured post work-up however this may be an artefact of the error in integration of the SFC analysis.

TABLE 29
Parr vessel reaction with PTFE stirrer bar and PTFE tape
on thermocouple-S/C 1,000/1, [S] = 0.6M, 114 g/L, MeOH,
0.1 eq. of NEt₃, 40° C., 5 bar H₂, 1500 rpm, 20.5 hours

| | Time | S.M. | P2 | P1 | Others | e.e. |
|---|---|---|---|---|---|---|
| Entry | (h) | (%) | (%) | (%) | (%) | (%) |
| 1 | 16 (temp. error) | 28 | 69 | 3 | 0 | 91 |
| 2 | 20.5 | <1 | 95 | 4 | 1 | 91 |
| 3 | 22.5 | 0 | 95 | 5 | 1 | 91 |
| 4 | After work-up* | 0 | 94 | 5 | 1 | 90 |

*Same work-up procedure as Table 23

### L. Scale Up to 300 mL Parr Vessel

Once it was established that there was a contaminant in the 25 mL Parr vessel which caused <90% e.e. to be obtained, the first scale-up in a 300 mL Parr vessel was carried out using S/C 200/1 in case there was also a background reaction caused by this vessel (Table 30). It was predicted that the fast reaction rate caused by the high loading would be able to provide a >90% e.e., by minimizing the impact from any background reaction which would have a much slower rate. (R)-Phanephos and [RuCl2(p-cym)]₂ (1.2:1 eq., 322 mg, 142 mg respectively) were weighed into a 300 mL Parr vessel followed by the substrate (17.87 g, 93 mmol). Methanol (155 mL, 0.6 M substrate concentration) was added to the vessel followed by triethylamine (1.3 mL, 9.3 mmol, 0.1 eq.). The vessel was sealed and purged with nitrogen 5 times (at ˜2 bar) and 5 times with stirring (˜500 rpm). The vessel was then purged with hydrogen 5 times (at ˜10 bar) and 5 times with stirring (˜500 rpm). The vessel was then pressurized to 5 bar hydrogen pressure and heated to 30° C. initially, then increased to 35° C. (with maximum stirring, >1500 rpm). The pressure was kept constant but with venting and refilling to 5 bar after sampling. After 5 hours, the vessel was allowed to cool. After 6 hours, the vessel was vented and purged with nitrogen. Each ˜0.1 mL sample was diluted to ˜1 mL with MeOH for SFC analysis.

The reaction was complete in 4-6 hours, with 91% e.e. of product. For the first 1.7 hours the temperature was ≤30° C., during which time consumption of hydrogen was recorded thus indicating the reaction can occur at <30° C. However, the temperature was increased and above 30° C. the reaction rate increased considerably, thus the temperature was increased to 35° C. and maintained until the reaction was complete. A high yield, with high purity (by 1H NMR), of the product was obtained after performing a work-up.

TABLE 30
300 mL Parr Vessel Scale Up-S/C 200/1,
[S] = 0.6M, 114 g/L, MeOH ,0.1 eq. of
NEt₃, 30-35° C., 5 bar H₂, >1500 rpm, 6 hours

| | Time | S.M. | P2 | P1 | Others | e.e. |
|---|---|---|---|---|---|---|
| Entry | (h) | (%) | (%) | (%) | (%) | (%) |
| 1 | 4 | 0.2 | 95 | 4 | <1 | 92 |
| 2 | 6 | <0.1 | 95 | 4 | <1 | 92 |
| 3 | After MeOH | <0.1 | 95 | 4 | <1 | 91 |
| | removal | | | | | |
| 4 | After work-up* | 0 | 95 | 4 | <1 | 91 |

*Work-up procedure: The contents of the Parr vessel were transferred into a round bottom flask using MeOH (10 mL) to wash the vessel and transfer the washings to the flask. MeOH was removed by concentrating under vacuum, followed by addition of EtOAc (40 mL) and 1M HCl (40 mL). Further portions of EtOAc (2 × 10 mL) and 1M HCl (10 mL) were used to wash the round bottom flask and transfer to the separating funnel. The funnel was shaken vigorously to mix the layers before allowing the layers to separate. The EtOAc organic layer was washed with further portions of 1M HCl (2 × 20 mL) and the aqueous layer was washed with further portions of EtOAc (2 × 20 mL) before the organic layers were combined. EtOAc was then removed under vacuum to leave behind the product as a greyish solid (17.5 g, 97% yield).

The second scale-up reaction carried out in the 300 mL Parr was carried out at S/C 1,000/1 (Table 31). At this point it was not known whether there was any contaminant in the vessel which would cause a lower e.e. value. The experiment was setup on the same substrate scale as the previous 300 mL reaction, except for catalyst loading ((R)-Phanephos and [RuCl₂(p-cym)]₂ (1.2:1 eq., 64 mg, 28 mg respectively)).

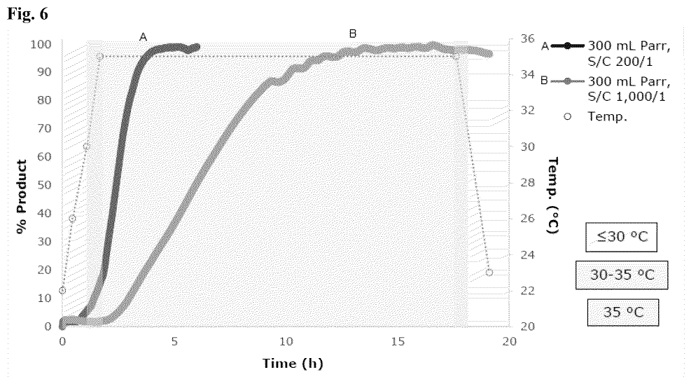

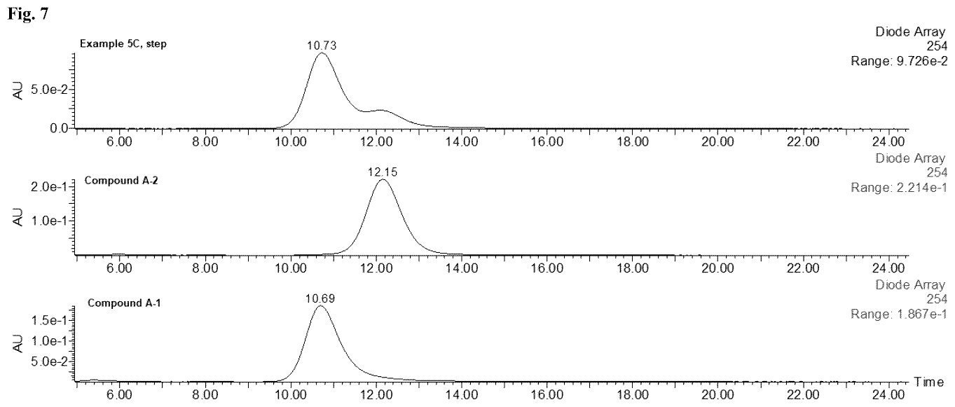

The results showed a significant amount of a background reaction as evidenced by the <90% e.e. value. From the hydrogen uptake, the reaction was signaled to be complete in ˜14 hours at S/C 1,000/1 instead of 4-6 hours as was seen when using S/C 200/1 (FIG. 6). This difference in reaction rate has meant that the background reaction has been allowed to have more impact on the e.e. value and therefore indicates the importance of evaluating each specific vessel with respect to the catalyst loading choice and desired e.e. outcome.

TABLE 31
300 mL Parr Vessel Scale Up-S/C 1,000/1,
[S] = 0.6M, 114 g/L, MeOH, 0.1 eq. of NEt₃,
30-35° C., 5 bar H₂, >1500 rpm, 19 hours

| | Time | S.M. | P2 | P1 | Others | e.e. |
|---|---|---|---|---|---|---|
| Entry | (h) | (%) | (%) | (%) | (%) | (%) |
| 1 | 17.5 | 0 | 91 | 8 | 1 | 83 |
| 2 | 19 | 0 | 89 | 10 | 1 | 80 |
| 3 | After MeOH | 0 | 92 | 8 | <1 | 84 |
| | removal | | | | | |
| 4 | After work-up* | 0 | 92 | 8 | <1 | 85 |

*Work-up procedure is the same as Table 30.

### M. Summary of Optimization

A key finding from this example, as shown in Table 32, was that the presence and quantity of a metal deposit contaminant in the reaction vessel caused an impact on decreasing the e.e. away from the maximum e.e. able to be obtained under the same conditions in a totally inert vessel. Increasing the catalyst loading for vessels in which a background reaction was observed was shown to be a way to overcome this effect on e.e. (entries 4-5).

TABLE 32
Summary of Best Conditions in Different Vessels-(R)-Phanephos + [RuCl₂(p-cym)]₂
(1.2:1 eq. of metal), [S] = 0.6M, MeOH, 0.1 eq. NEt₃, 5 bar H₂, 30-40° C.

| | Substrate | | | Vessel Type & | S.M. | P2 | P1 | Others | e.e. |
|---|---|---|---|---|---|---|---|---|---|
| Entry | scale | | s/c | Amount of MeOH | (%) | (%) | (%) | (%) | (%) |
| 1 | 192 | mg | 1,000/1 | Endeavor | 0 | 96 | 3 | 0 | 93 |
| | | | | (1.7 mL) | | | | | |
| 2 | 1.6 | g | 1,000/1 | Stainless steel | 0 | 92 | 7 | 1 | 85 |
| | | | | Parr (14 mL) | | | | | |
| 3 | 1.8 | g | 1,000/1 | Glass-lined | 0 | 94 | 5 | 1 | 90 |
| | | | | Parr (16 mL) | | | | | |
| 4 | 17.9 | g | 200/1 | Stainless steel | 0 | 95 | 4 | 1 | 91 |
| | | | | Parr (155 mL) | | | | | |
| 5 | 17.9 | g | 1,000/1 | Stainless steel | 0 | 92 | 8 | 1 | 85 |
| | | | | Parr (155 mL) | | | | | |

This example focused on optimizing the conditions for using S/C 1,000/1 of (R)-Phanephos+[RuCl₂(p-cym)]₂ to give >90% of P2 (desired product enantiomer). Encouragingly, the reaction conditions were found to be successful at 5 bar H2 pressure. Thus, the optimization was carried out using S/C 1,000/1 and 5 bar pressure. This included a DoE study to investigate the effect of parameters: substrate concentration, amount of triethylamine and temperature.

Increasing the substrate concentration had the biggest effect on decreasing the conversion and e.e. values obtained. Reducing the amount of triethylamine used to 0.1 eq. (w.r.t. substrate) was found to be successful in allowing full conversion with >90% e.e. for 0.6 M substrate concentration. Using temperatures of 30-40° C. were also found to help with achieving maximum e.e. values.

The optimized conditions found on small scale were then transferred to standalone Parr vessels, to demonstrate the hydrogenation reaction on larger scale. Four different vessels have been used (Endeavor, 25 mL stainless steel Parr, 50 mL glass-lined Parr and 300 mL stainless steel Parr) in this work and it has been found that there can be variation in the e.e. value obtained in different vessels caused by the presence or absence of a non-enantioselective background reaction. To overcome this issue of achieving <90% e.e., it has been shown that S/C 200/1 is a sufficient loading to compensate for the presence of any background reaction. Alternatively, an inert vessel (i.e. glass-lined) demonstrated >90% e.e. can be achieved using S/C 1,000/1.

### Example 3. Chiral Synthesis of Compounds A-1 and A-2

### A. Synthesis of P2

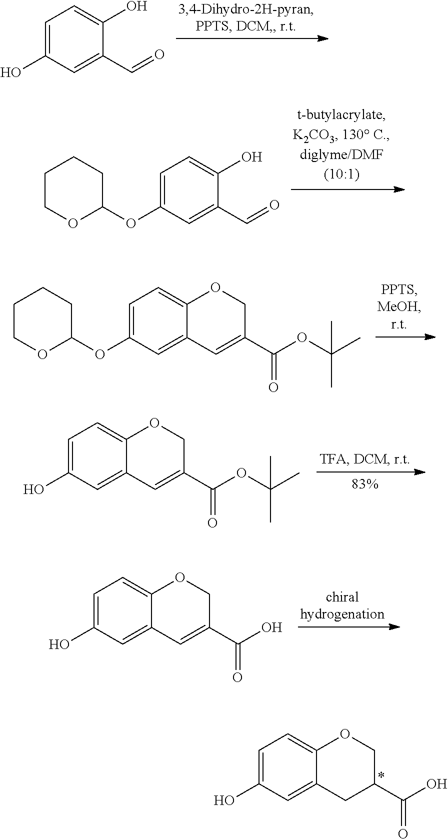 [SMILES: O=Cc1cc(O)ccc1O ; O=Cc1cc(OC2CCCCO2)ccc1O ; CC(C)(C)OC(=O)C1=Cc2cc(OC3CCCCO3)ccc2OC1 ; CC(C)(C)OC(=O)C1=Cc2cc(O)ccc2OC1 ; O=C(O)C1=Cc2cc(O)ccc2OC1 ; O=C(O)C1COc2ccc(O)cc2C1]

Step 1: To a solution of 2,5-dihydroxybenzaldehyde (200 g, 1448 mmol) and pyridinium p-toluenesulfonate (18.2 g, 72.4 mmol) in DCM (3.75 L) was added 3,4-dihydro-2H-pyran (165 mL, 1810 mmol) dropwise over 10 minutes and the reaction temperature warmed to 30° C. The reaction was stirred for 2 hours and checked by UPLC-MS which indicated the reaction was 92% complete (˜5% starting material and ˜3% later running unknown). The reaction was stopped. The reaction was washed with water (1.5 L) and the DCM solution was passed through a 750 g silica pad and followed through by DCM (2.5 L). The DCM solution was reduced in-vacuo and the crude product was then slowly diluted with Pet. Ether to ˜1 L total volume, stirred and cooled to ˜10° C. to afford a thick yellow slurry. The product was filtered and washed with Pet. Ether (2×150 mL) and pulled dry for 3 hours to afford 2-hydroxy-5-tetrahydropyran-2-yloxy-benzaldehyde (265 g, 1192 mmol, 82% yield) as a bright yellow solid. 1H NMR (400 MHz, DMSO-d₆) δ/ppm: 10.35 (s, 1H), 10.23 (s, 1H), 7.32-7.19 (m, 2H), 6.94 (d, J=8.9 Hz, 1H), 5.36 (t, J=3.3 Hz, 1H), 3.77 (ddd, J=11.2, 8.8, 3.6 Hz, 1H), 3.59-3.49 (m, 1H), 1.94-1.45 (m, 6H). UPLC-MS (ES+, Short acidic): 1.64 min, m/z 223.0 [M+H]⁺ (100%).

Step 2:2-hydroxy-5-tetrahydropyran-2-yloxy-benzaldehyde (107 g, 481 mmol) was dissolved in diglyme (750 mL) and K₂CO₃ (133 g, 963 mmol) was added on one portion with stirring to afford a bright yellow suspension. The reaction was then heated to 140° C. and tert-butyl acrylate (155 mL, 1059 mmol) in DMF (75 mL) was added over 10 minutes starting at ˜110° C. and up to 130° C. Maintained this temperature for a further 1 hour. UPLC-MS indicated that the reaction had progressed 75%. After a further hour this showed clean conversion to 85% product and little or no side-products. After another 3 hours UPLC-MS showed 88% product (previous reactions had showed that further heating did not afford more conversion). The dark brown reaction was cooled to room temperature overnight and filtered to remove inorganics. The reaction was suspended in EtOAc (2.5 L) and water (2.5 L) and the phases separated. The aqueous was re-extracted with EtOAc (2.5 L) and the combined organics were washed with brine (2×1.5 L) and the organics were reduced in-vacuo. The crude product was then purified on silica (2 Kg) loading in a minimum volume of DCM. A gradient of EtOAc in Pet. Ether (10-25%) was run and clean product fractions combined and reduced in-vacuo to afford tert-butyl 6-tetrahydropyran-2-yloxy-2H-chromene-3-carboxylate (93.5 g, 281 mmol, 58% yield) as a yellow solid. 1H NMR (400 MHz, DMSO-d₆) δ/ppm: 7.37 (q, J=1.2 Hz, 1H), 7.05 (d, J=2.9 Hz, 1H), 6.94 (dd, J=8.8, 2.9 Hz, 1H), 6.79 (dd, J=8.7, 0.7 Hz, 1H), 5.35 (t, J=3.3 Hz, 1H), 4.82 (d, J=1.4 Hz, 2H), 3.77 (ddt, J=13.3, 8.3, 4.2 Hz, 1H), 3.59-3.48 (m, 1H), 1.93-1.49 (m, 6H), 1.49 (s, 9H). UPLC-MS (ES+, Short acidic): 2.18 min, m/z ([M+H]⁺) not detected (100%).

Step 3: tert-butyl 6-tetrahydropyran-2-yloxy-2H-chromene-3-carboxylate (215 g, 647 mmol) was suspended in MeOH (1.6 L) at room temperature (did not dissolve immediately) and pyridinium p-toluenesulfonate (16.3 g, 64.7 mmol) added. The reaction was warmed to 40° C. with a hot water bath and checked by UPLC-MS for progress after 1 hour which indicated the reaction was complete and was a clear orange solution. The reaction was reduced in-vacuo and the crude product dissolved in DCM (2 L) and washed with water (1 L). The organic layer was dried (MgSO₄), filtered and reduced in-vacuo to afford the crude product as a yellow solid. This was suspended in Pet. Ether and stirred in an ice bath before filtering, to afford a bright yellow solid. This was dried under high vac at 50° C. for 2 hours to afford tert-butyl 6-hydroxy-2H-chromene-3-carboxylate (144.4 g, 582 mmol, 90% yield). ¹H NMR (400 MHz, DMSO-d₆) δ/ppm: 9.17 (s, 1H), 7.33 (s, 1H), 6.76-6.64 (m, 3H), 4.77 (d, J=1.4 Hz, 2H), 1.49 (s, 9H). UPLC-MS (ES+, Short acidic): 1.71 min, m/z 247.2 [M−H]− (100%).

Step 4: tert-Butyl 6-hydroxy-2H-chromene-3-carboxylate (84.g, 338.34 mmol) was dissolved in DCM (500 mL) and trifluoroacetic acid (177.72 mL, 2320.9 mmol) added at room temperature and the reaction stirred to give a brown solution. Initially gas evolution was noted and the reaction was stirred over several days at room temperature. DCM and TFA were removed in-vacuo and finally azeotroped with 200 ml of toluene before slurrying with diethyl ether and filtering to give the crude product 6-hydroxy-2H-chromene-3-carboxylic acid (53.15 g, 276.58 mmol, 81.745% yield) as a cream solid. 1H NMR (400 MHz, DMSO-d₆) δ/ppm: 12.77 (s, 1H), 9.14 (s, 1H), 7.37 (t, J=1.4 Hz, 1H), 6.72 (dd, J=2.4, 0.9 Hz, 1H), 6.70-6.64 (m, 2H), 4.78 (d, J=1.4 Hz, 2H).

Step 5: (R)-Phanephos and [RuCl₂(p-cym)]₂ (1.2:1 eq., 6.6 mg, 3.0 mg respectively) were weighed into a 50 mL glass lined Parr vessel followed by the substrate (1.845 g, 9.6 mmol). Methanol (16 mL, 0.6 M substrate concentration) was added to the vessel followed by triethylamine (135 μL, 0.96 mmol, 0.1 eq.). A PTFE stirrer bar was added and the thermocouple was covered with PTFE tape. The vessel was sealed and purged with nitrogen 5 times (at ˜2 bar) and 5 times with stirring (˜500 rpm). The vessel was then purged with hydrogen 5 times (at ˜10 bar) and 5 times with stirring (˜500 rpm). The vessel was then pressurised to 5 bar hydrogen pressure and heated to 40° C. (with 1500 rpm stirring speed). The pressure was kept constant but with venting and refilling to 5 bar after sampling. After 21.5 hours, the vessel was allowed to cool. After 22.5 hours, the vessel was vented and purged with nitrogen. Each ˜0.1 mL sample was diluted to ˜1 mL with MeOH for SFC analysis. Work-up procedure: MeOH removed by concentrating under vacuum, followed by addition of EtOAc (10 mL) and 1 M HCl (10 mL). The layers were mixed before separating. The EtOAc layer was washed with a further portion of 1 M HCl (4 mL) before removing the aqueous layer to leave the EtOAc organic phase. The aqueous layer was then washed with a further portion of EtOAc (4 mL) and the organic layers were combined. EtOAc was then removed under vacuum to leave behind the product as a greyish solid (See Table 29). P2 is the first eluting product with a retention time of 5.8 min and P1 is the second eluting product with a retention time of 6.1 min using the SFC method as described in Example 1.

### B. Synthesis of 5-fluoro-3,4-dihydro-1,8-naphthyridin-2 (1H)-one

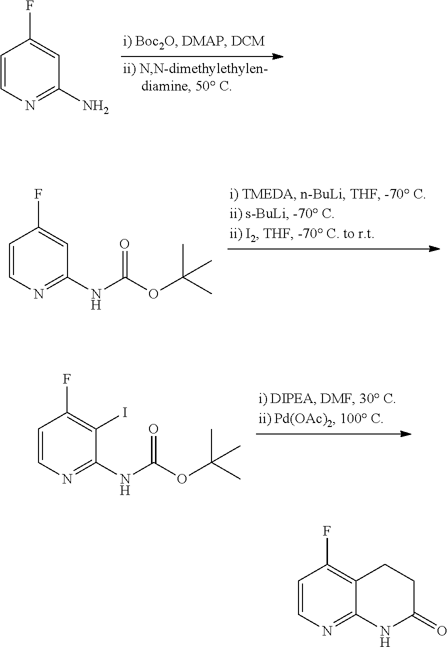 [SMILES: Nc1cc(F)ccn1 ; CC(C)(C)OC(=O)Nc1cc(F)ccn1 ; CC(C)(C)OC(=O)Nc1nccc(F)c1I ; O=C1CCc2c(F)ccnc2N1]

Step 1:2-Amino-4-fluoropyridine (400 g, 3568 mmol) was charged into a 10 L fixed reactor vessel and then taken up in DCM (4 L) as a slurry under nitrogen atmosphere. To this was added DMAP (43.6 g, 357 mmol) and cooled to 10° C. Di-tert-butyldicarbonate (934 g, 4282 mmol) was added, as a solution in DCM (1 L), over the space of 1.5 hours. The reaction was stirred at room temperature for 2 hours after which time the complete consumption of the starting material was evident by NMR. To the reaction was added N,N-dimethylethylenediamine (390 mL, 3568 mmol) and the reaction warmed to 40° C. overnight (converting any di-BOC material back to the mono-BOC desired product). Allowed to cool to room temperature and then diluted with further DCM (2 L) and washed with water (2 L). Extracted with further DCM (2 L), washed with water (1 L), brine (1.2 L) and dried (MgSO₄) before filtering. The solvents were removed in-vacuo and the resultant product was slurried in DCM/Pet. Ether (1:1)(500 mL). Filtered, washed with further Pet. Ether and pulled dry to afford tert-butyl N-(4-fluoro-2-pyridyl) carbamate (505 g, 2380 mmol, 67% yield) as a cream solid product. A second crop of material was isolated from the mother liquors after passing through a short pad of silica followed by trituration with DCM/Pet. Ether (1:1)(˜200 mL) to afford tert-butyl N-(4-fluoro-2-pyridyl) carbamate (46.7 g, 220 mmol, 6% yield). 1H NMR (400 MHz, DMSO-d₆) δ/ppm: 10.13 (d, J=1.7 Hz, 1H), 8.26 (dd, J=9.4, 5.7 Hz, 1H), 7.60 (dd, J=12.3, 2.4 Hz, 1H), 6.94 (ddd, J=8.2, 5.7, 2.4 Hz, 1H), 1.47 (s, 9H). UPLC-MS (ES+, Short acidic): 1.64 min, m/z 213.1 [M+H]+ (98%).

Step 2: tert-butyl-N-(4-fluoro-2-pyridyl) carbamate (126 g, 594 mmol) and TMEDA (223 mL, 1484 mmol) were taken up in dry THF (1.7 L) and then cooled to −78° C. under nitrogen atmosphere. To this solution was added n-butyllithium solution (2.5M solution in hexanes)(285 mL, 713 mmol) and then allowed to stir for a further 10 minutes. sec-Butyllithium solution (1.2M in cyclohexane)(509 mL, 713 mmol) was added keeping the reaction temperature below −70° C. whilst stirred for 1 hour. After this time, Iodine (226 g, 891 mmol) in THF (300 mL) was added slowly and dropwise over 30 minutes to keep the temp below −65° C. Stirred at −70° C. for another 10 minutes and then quenched by the addition of sat. aq. NH₄Cl solution (400 mL) and then a solution of sodium thiosulphate (134 g, 848 mmol) dissolved in water (600 mL). This addition raised the temperature to ˜−25° C. The reaction was warmed to room temperature then transferred to the 5 L separator and extracted with EtOAc (2×1.5 L) and then washed with brine (500 mL), dried (MgSO₄) and then evaporated in vacuo to afford crude material (˜200 g). This was taken up in hot DCM (500 mL)(slurry added to the silica pad) and then passed through a 2 Kg silica pad. Washed through with DCM (10×1 L fractions) and then the product was eluted from the column with EtOAc in Pet. Ether (10% to 100%), (1 L at each 10% increase, with 1 L fractions). This gave 2 mixed fractions and clean product containing fractions, which were combined and evaporated in vacuo to afford tert-butyl N-(4-fluoro-3-iodo-2-pyridyl) carbamate (113.4 g, 335.4 mmol, 57% yield) as a white solid. Clean by UPLC-MS and NMR. The mixed fractions were combined with previous crude material to afford 190 g in total of a cream solid that was composed of ˜50% of the desired product. This was re-columned as above to afford a combined second crop from all 4 batches as a cream solid tert-butyl N-(4-fluoro-3-iodo-2-pyridyl) carbamate (107.5 g, 318 mmol, 54% yield). 1H NMR (400 MHz, DMSO-d₆) δ/ppm: 9.47 (s, 1H), 8.33 (dd, J=8.7, 5.5 Hz, 1H), 7.19 (dd, J=7.3, 5.5 Hz, 1H), 1.46 (s, 9H). UPLC-MS (ES+, Short acidic): 1.60 min, m/z 339.1 [M+H]+ (100%).

Step 3: tert-butyl N-(4-fluoro-3-iodo-2-pyridyl) carbamate (300 g, 887 mmol), 3,3-dimethoxyprop-1-ene (137 mL, 1153 mmol) and DIPEA (325 mL, 1863 mmol) were suspended in DMF (2 L) and water (440 mL) to give a yellow slurry. This was degassed for 20 minutes at 30° C. To this mixture was then added Palladium (II) acetate (19.92 g, 89 mmol) in one portion and degassed again for a further 15 mins. The reaction was slowly and carefully heated to 100° C. Gas evolution at around 85° C. (large volumes of off gassing, presumably due to the loss of Boc group as CO₂ and isobutylene). The reaction became darker once off gassing finished and full solubility achieved. The reaction was then heated at 100° C. for 3 hours and checked by UPLC-MS (70% desired product, 18% un-cyclised intermediate and 7% des-iodo BOC). The reaction was heated for a further 2 hours and this showed 81% desired product, 12% un-cyclised intermediate and 8% des-iodo BOC. After 7 hours the reaction showed 89% desired product, 4% un-cyclised intermediate and 7% des-iodo BOC. The reaction was heated overnight. The reaction solution was cooled and filtered through celite and evaporated in-vacuo to a thick dark orange slurry which was then suspended in water (1 L) and acidified to pH˜1-2 with aq. HCl (4N) solution. This was then basified to pH˜9 with sat. aq. NaHCO₃ solution. Extracted with DCM (2×2 L) and washed with brine and dried (MgSO₄). EtOAc (2 L) was added to the solution and then the organics were passed through a 500 g silica plug. This was then followed by DCM/EtOAc (1:1)(2 L) and finally EtOAc (2 L)(the final wash through contained only baseline). The product containing fractions were combined and reduced in-vacuo to give an orange slurry and then suspended in hot diethyl ether (300 mL), cooled back to ˜10° C. in an ice bath with stirring before being filtered and washed with 150 mL of ice cold diethyl ether. Pulled dry to afford 5-fluoro-3,4-dihydro-1H-1,8-naphthyridin-2-one (58.4 g, 351.5 mmol, 39.6% yield) as a cream fluffy solid. 1H NMR (400 MHz, DMSO-d₆) δ/ppm: 10.69 (s, 1H), 8.29-7.90 (m, 1H), 6.92 (dd, J=8.8, 5.7 Hz, 1H), 2.88 (dd, J=8.3, 7.1 Hz, 2H), 2.57-2.47 (m, 2H). UPLC-MS (ES+, Short acidic): 1.04 min, m/z 167.0 [M+H]+ (100%).

### C. Synthesis of Compounds A-1 and A-2

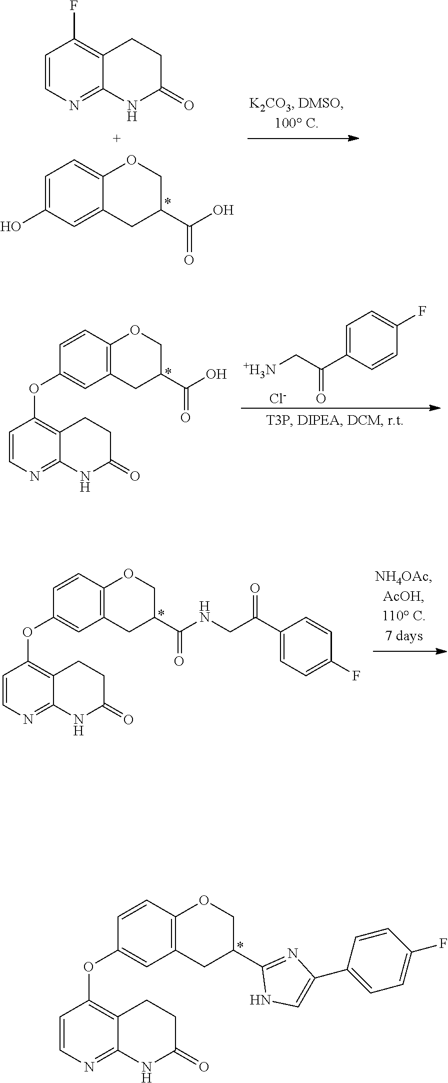

Step 1: Potassium carbonate (832 mg, 6.02 mmol) was added to a stirred solution of 5-fluoro-3,4-dihydro-1H-1,8-naphthyridin-2-one (250 mg, 1.5 mmol), P2 (see step A, 292 mg, 1.5 mmol; 85% ee) and DMSO (2 mL) at room temperature. The reaction was degassed and flushed with nitrogen 3 times before being stirred under a nitrogen atmosphere for 18 hours at 100° C. The reaction mixture was cooled to room temperature and diluted with water (20 mL) and the resulting mixture extracted with EtOAc (20 mL). A solution of citric acid (1156.3 mg, 6.02 mmol) in water (10 mL) was then added to the aqueous layer resulting in a solid precipitate which was filtered and dried in vacuo to give (S)- or (R)-6-[(7-oxo-6,8-dihydro-5H-1,8-naphthyridin-4-yl)oxy]chromane-3-carboxylic acid (345 mg, 1.01 mmol, 67% yield) as a white solid. UPLC-MS (ES+, Short acidic): 1.29 min, m/z 341.1 [M+H]+. 1H NMR (400 MHz, DMSO-d₆) δ/ppm: 12.71 (1H, br s), 10.47 (1H, s), 7.95 (1H, d, J=6.0 Hz), 6.97 (1H, d, J=2.4 Hz), 6.89 (1H, dd, J=8.4 Hz, 2.4 Hz), 6.83 (1H, d, J=8.4 Hz), 6.24 (1H, d, J=6.0 Hz), 4.33 (1H, dd, J=11.2 Hz, 3.2 Hz), 4.15 (1H, dd, J=11.2 Hz, 7.2 Hz), 3.05-2.89 (5H, m), 2.53 (2H, t, J=7.6 Hz).

Step 2: Propylphosphonic anhydride (0.91 mL, 1.52 mmol) was added to a stirred solution of (S)-6-[(7-oxo-6,8-dihydro-5H-1,8-naphthyridin-4-yl)oxy]chromane-3-carboxylic acid (345 mg, 1.01 mmol), 2-amino-1-(4-fluorophenyl) ethanone hydrochloride (288 mg, 1.52 mmol), N,N-diisopropylethylamine (0.88 mL, 5.07 mmol) and DCM (10 mL) at room temperature. After stirring for 2 hours the reaction was complete by LCMS. Water (50 mL) and DCM (50 mL) were added and the organic layer separated and washed with sat. aq. NaHCO₃ (50 mL). The organic layer was dried over sodium sulfate and solvent removed in vacuo. The residue was purified by column chromatography using an eluent of 0-5% MeOH in DCM to give (S)- or (R)—N-[2-(4-fluorophenyl)-2-oxo-ethyl]-6-[(7-oxo-6,8-dihydro-5H-1,8-naphthyridin-4-yl)oxy]chromane-3-carboxamide (300 mg, 0.63 mmol, 62% yield) as a yellow solid. UPLC-MS (ES+, Short acidic): 1.52 min, m/z 476.4 [M+H]+. ¹H NMR (400 MHz, DMSO-d₆) δ/ppm: 10.47 (1H, s), 8.60-8.54 (1H, m), 8.08 (1H, dd, J=8.8 Hz, 5.6 Hz), 7.95 (1H, d, J=5.6 Hz), 7.41-7.37 (2H, m), 7.01-6.97 (1H, m), 6.90 (1H, dd, J=8.8 Hz, 3.2 Hz), 6.86 (1H, d, J=8.8 Hz), 6.25 (1H, d, J=5.6 Hz), 4.65 (2H, d, J=6.0 Hz), 4.42-4.35 (1H, m), 3.96 (1H, t, J=9.6 Hz), 3.03-2.87 (5H, m), 2.55-2.52 (2H, m), 1 exchangeable proton not seen.

Step 3: (S)- or (R)—N-[2-(4-fluorophenyl)-2-oxo-ethyl]-6-[(7-oxo-6,8-dihydro-5H-1,8-naphthyridin-4-yl)oxy]chromane-3-carboxamide (300 mg, 0.63 mmol), ammonium acetate (1216 mg, 15.77 mmol) and acetic acid (5 mL) were combined in a sealable vial, the vial sealed and the reaction stirred and heated to 130° C. for 18 hours after which time the reaction was complete by LCMS. The reaction was cooled to room temperature and AcOH removed in vacuo. DCM (50 mL) was added to the residue and sat. aq. NaHCO₃ (50 mL) added. The organic layer was separated and washed with brine, dried over sodium sulfate and solvent removed in vacuo. The residue was purified by column chromatography using an eluent of 0-10% MeOH in DCM to give (R)- or (S)-5-[3-[4-(4-fluorophenyl)-1H-imidazol-2-yl]chroman-6-yl]oxy-3,4-dihydro-1H-1,8-naphthyridin-2-one (141 mg, 0.31 mmol, 49% yield) as a yellow solid.

Chiral LCMS of the product, together with chiral LCMS's of Compounds A-1 and A-2 showed that this product is predominantly Compounds A-1 (FIG. 7), with a similar ee to that of the starting acid (85% ee), however accurate analysis cannot be done due to overlap of the peaks. UPLC-MS (ES+, Short acidic): 1.36 min, m/z 457.2 [M+H]+. ¹H NMR (400 MHz, DMSO-d₆) δ/ppm: 12.31 (0.2H, s), 12.10 (0.8H, s), 10.47 (1H, s), 7.96 (1H, d, J=6.0 Hz), 7.80-7.75 (1.8H, m), 7.69-7.65 (0.2H, m), 7.59-7.78 (0.8H, m), 7.29-7.23 (0.4H, m), 7.19-7.13 (1.8H, m), 7.03-7.00 (1H, m), 6.92 (1H, dd, J=8.8 Hz, 2.8 Hz), 6.89 (1H, d, J=8.8 Hz), 6.27 (1H, d, J=6.0 Hz), 4.55-4.48 (1H, m), 4.16-4.09 (1H, m), 3.44-3.36 (1H, m), 3.30-3.21 (1H, m), 3.16-3.09 (1H, m), 2.94 (2H, t, J=7.2 Hz), 2.54 (2H, t, J=7.2 Hz).

### Chiral LCMS:

Chiracel OZ-RH
150 mm×4.6 mm, 5 μm
Mobile phase A: 20 mM ammonium bicarbonate
Mobile phase B: acetonitrile
Isocratic 1.2 ml/min
50% A; 50% B
Samples diluted in methanol (1 mg/ml)

Synthesis to prepare predominantly Compound A-2 can be carried out using P1 instead of P2 (see Step A).

Enantiomers of the product can be separated using the following conditions:
Instrument: Thar 200 preparative SFC (SFC-7)
Column: ChiralPak AS, 300×50 mm I.D., 10 μm
Mobile phase: A for CO₂ and B for Ethanol
Gradient: B 50%
Flow rate: 200 mL/min
Back pressure: 100 bar
Column temperature: 38° C.
Wavelength: 220 nm
Cycle time: ˜ 5 min

### Example 4. Large Scale Chiral Synthesis of Compounds A-1 and A-2

Liquid chromatography-mass spectrometry: Unless otherwise noted, following ultra-performance LCMS method and parameters were used to characterize products of each step described in this example.
Instrument: Waters H Class UPLC with QDA detector
Column: Acquity UPLC BEH C18 2.1×100 mm, 1.7 μm column, PN: 186002352
Wavelength: UV 210 nm
Column temperature: 30° C.
Sampler temperature: 20° C.
Flow rate: 0.3 mL/min
Injection volume: 1 μL
Mobile phase: A: 10 mM NH₄OAc in water
B: ACN:MeOH=8:2 (v/v)
Gradient program:

| time | | |
|---|---|---|
| (min) | A % | B % |
| 0.00 | 95 | 5 |
| 3.00 | 95 | 5 |
| 8.00 | 65 | 35 |
| 15.00 | 55 | 45 |
| 18.00 | 5 | 95 |
| 21.00 | 5 | 95 |
| 21.10 | 95 | 5 |
| 25.00 | 95 | 5 |

Run time: 25.0 min

| General: | Ion source | QDA | |
|---|---|---|---|
| Signal | Mode | MS2 Scan | |
| setting: | lon Range | m/z = 30~m/z = 800 | |
| | Polarity | Positive and Negative | |
| | Probe Temperature | 600° | C. |
| | Capillary Voltage | 800 | V |

### A. Synthesis of P2

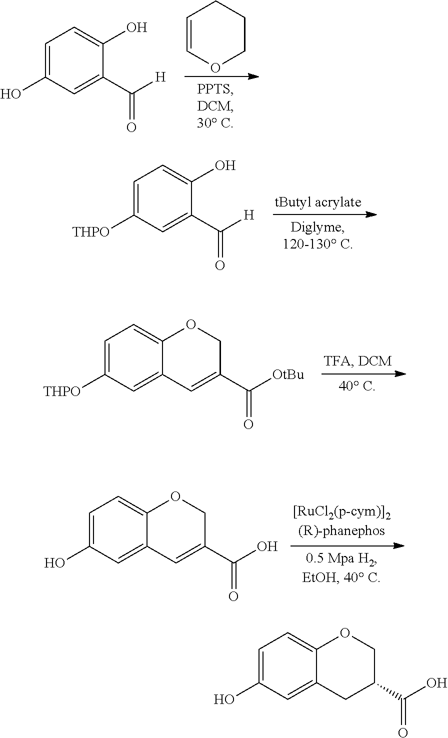

Step 1: 2,5-Dihydroxybenzaldehyde (13.6 kg, 98.18 mol) was dried using 2× azeotropic concentrations with 2×125-130 kg of THF at up to 35° C., concentrating under vacuum to 27-41 kg each time. The THF was then removed using 4×azeotropic concentrations with 4×179-187 kg of DCM at up to 35° C., concentrating under vacuum to 27-41 kg each time. The concentrate was diluted with DCM (284 kg) and pyridine p-toluenesulfonate (PPTS; 1.25 kg, 4.97 mol) was added. 3,4-dihydro-2H-pyran (10.4 kg, 123.63 mol) was added slowly at between 25-35° C. and the reaction was stirred at 30° C. for 90 minutes. The mixture was added to a solution of Na₂CO₃ (7.1 kg) in water (138 kg) at −15° C. and allowed to warm to 25° C. and then stirred for 6 h. The mixture was filtered through Celite® (33 kg), washing with DCM (92.5 kg). The filtrate was allowed to stand for 1 h and then the organic phase was separated and concentrated to 27-41 kg. The DCM was then removed using 3×azeotropic concentrations with 3×105 kg n-heptane at up to 35° C., concentrating under vacuum to 27-41 kg each time. The concentrate was diluted with n-heptane (210 kg) and the heated to 30-40° C. and stirred for 6 h. The solution was then cooled to −5 to ˜15° C. over 4 h, stirred for 9 h and filtered, washing the filter cake with n-heptane (39.5 kg). The wet cake was dried at 30-40° C. for 24 h in vacuo to give 2-hydroxy-5-(oxan-2-yloxy)benzaldehyde (9.38 kg, 40.6%). Additional product (8.00 kg, 34.3%) was recovered by dissolving solid attached to the walls of the reaction vessel with 42 kg DCM and concentrating the resultant solution in vacuo to give a further 8.00 kg (34.3% yield) of product to give a total yield of 74.9% (17.38 kg). LCMS (ES−): 15.18 min, m/z 221.12 [M−H]−.

Step 2: To a stirring solution of 2-hydroxy-5-(oxan-2-yloxy)benzaldehyde (16.95 kg, 76.27 mol) in diglyme (113.4 kg) was added K₂CO₃ (21.4 kg, 154.83 mol) and the mixture was heated to between 80-90° C. Tert-butyl prop-2-enoate (20.0 kg, 156.04 mol) was added, and the mixture was heated to between 120-130° C. and stirred for 18 hr. The mixture was cooled and filtered, and the filter cake washed with EtOAc (80.0 kg). The filtrate was diluted with EtOAc (238.0 kg) and water (338.0 kg) and stirred for 1 hr at 20-30° C., then stood for 2 hr. The mixture was filtered through Celite® (40.0 kg), and the filter cake washed with EtOAc (84.0 kg). The filtrate was left to stand for 2 hr and the aqueous layer was extracted with EtOAc (312.0 kg), stirring for 1 hr at 0-30° C. and standing for 2 hr. The organic layers were combined and washed with 2×345 kg water, stirring at between 20-30° C. for 1 hr and standing for 2 hr for each wash. The combined organics were then concentrated to 182.4 kg maintaining the temperature below 50° C. under vacuum. This gave the product tert-butyl 6-(oxan-2-yloxy)-2H-chromene-3-carboxylate as a 9.3% solution in diglyme/EtOAc (66.9% yield) and was used in the next stage without further isolation. LCMS (ES−): 20.26 min, m/z 247.12 [M−THP]−.

Step 3: Tert-butyl 6-(oxan-2-yloxy)-2H-chromene-3-carboxylate (16.9 kg, 50.84 mol) as a 181.8 kg solution in diglyme/EtOAc was concentrated to 68 kg under vacuum at 50° C. TFA (110.3 kg, 1002.46 mol) was added and the reaction was warmed to 40° C. under nitrogen flow and then stirred for 8 hrs. The mixture was then diluted with DCM (222.0 kg) and cooled to between −5 and −15° C., and then stirred for 7 hrs. The solid was filtered and the filter cake washed with DCM (67.0 kg). The wet cake was dried for 24 hr under vacuum at between 30-40° C. to give 6-hydroxy-2H-chromene-3-carboxylic acid (8.75 kg, 78.5% yield). LCMS (ES−): 0.85 min, m/z 191.11 [M−H]−.

Step 4: To a stirring solution of 6-hydroxy-2H-chromene-3-carboxylic acid (7.19 kg, 37.4 mol) in N₂-degassed EtOH (60 kg) was added (R)-Phanephos (131 g, 0.227 mol), [RuCl₂(p-cym)]₂ (70 g, 0.114 mol), and Et₃N (5.6 kg, 55.3 mol). The reaction atmosphere was replaced with 3×N₂ and then 3×H2, adjusting the H2 pressure to between 0.5-0.6 MPa, and then stirred for 18 hrs at 40° C. The atmosphere was then replaced with 3×N₂ and then 3×H2, adjusting the H2 pressure to between 0.5-0.6 MPa again and the mixture was stirred for a further 18 hrs.

The mixture was concentrated in vacuo to ca. 30 kg at no more than 40° C. The reaction was diluted with MTBE (53 kg) and cooled to between 15-25° C. 5% Na₂CO₃ (80 kg) was added dropwise, and the mixture was stirred for 2 hrs and stood for 2 hrs at between 15-25° C. The aqueous layer was collected and 5% Na₂CO₃ (48 kg) was added to the organic layer, then stirred for 2 hrs at 15-25° C. and filtered through Celite® (10.0 kg). The wet cake was washed with water (20 kg) and the combined aqueous filtrate and aqueous layer were diluted with IPAc (129.0 kg). The pH of the mixture was adjusted to 1-3 with dropwise addition of 6 N HCl (29 kg) at 15-25° C. and stirred for 2 hrs. The mixture was filtered through Celite® (10 kg), washing the filter cake with IPAc (34 kg) and the filtrate was left to stand for 2 hrs at 15-25° C. The aqueous layer was then extracted with IPAc (34 kg) and the combined organic layers were concentrated to ca. 35 kg under vacuum at no more than 40° C. Me-cyclohexane (21 kg) was added dropwise at 15-25° C. and concentrated to ca. 35 kg under vacuum at no more than 40° C. Further Me-cyclohexane (20 kg) was added dropwise at 15-25° C. and stirred for 3 hrs. The mixture was then stirred at 40-50° C. for 4 hrs and cooled to 15-25° C. over 3 hrs and then stirred for a further 2 hrs.

The mixture was then filtered, washing the filter cake with 16.4 kg of IPAc/Me-cyclohexane (1/4, v/v). The wet cake was dried for 24 hrs at 35-45° C. under vacuum to give (3R)-6-hydroxy-3,4-dihydro-2H-1-benzopyran-3-carboxylic acid (5.2 kg, 68.6% yield, chiral purity 95.5%). Further product was isolated by rinsing solid from the reaction vessel wall with EtOH (42 kg) and concentrating to dryness. The resulting solid was suspended in IPAc (875 mL) and Me-cyclohexane (2625 mL) and stirred for 5 h at 40° C. and then cooled to 20° C. over 2 h and stirred for 16 h and filtered. The filter cake was then split into 2 equal batches and each batch suspended in IPAc (912 mL) and Me-cyclohexane (2737 mL). The resulting mixtures were stirred at 45° C. for 18 h and then filtered and the filter cake dried at 45° C. to give (3R)-6-hydroxy-3,4-dihydro-2H-1-benzopyran-3-carboxylic acid (1.27 kg, 17% yield, chiral purity 96.2%). LCMS (ES−): 1.74 min, m/z 193.03 [M−H]−.

### Chiral Resolution to Improve Chiral Purity:

(3R)-6-Hydroxy-3,4-dihydro-2H-1-benzopyran-3-carboxylic acid (P2; 5.94 kg, 30.59 mol) (chiral purity=95.5%) was dissolved in IPAc (138.2 kg) and stirred for 2 hrs at 20-30° C. The solution obtained was filtered through Celite® (12 kg), washing through with IPAc (25 kg). In a separate vessel, (S)-(+)-2-phenylglycinol (4.4 kg, 32.07 mol) was dissolved in IPAc (56 kg), stirring for 1 hr at 40-50° C. The filtrate was added to this solution over 4 hrs at 40-50° C., and stirred for 1 hr. The mixture was then stirred for 1 hr at 15-25° C., and concentrated to ca. 120 kg under vacuum at no more than 40° C. The concentrate was stirred for 3 hrs at 15-25° C. and filtered, washing through with IPAc (12 kg). (chiral purity=96.2%).

The wet cake was redissolved in EtOH (29 kg), heated to 40-50° C. and diluted with IPAc (64 kg). 30 g of dry product was added and stirred for 30 min at 15-25° C. The mixture was concentrated to ca. 42 kg under vacuum at no more than 40° C., and rediluted with IPAc (64 kg). This step was repeated two additional times, then stirred at 40-50° C. for 8 hrs. The mixture was filtered, washing through with IPAc (13 kg) (chiral purity=97.7%). This recrystallisation process was repeated two further times, for a total of 3 recrystallisation rounds to give material with 98.9% chiral purity.

The wet cake (10.7 kg) was then dissolved in 1N HCl (45.4 kg) and stirred for 1 hr at 20-30° C. The mixture was filtered through Celite® (11.5 kg), washing through with IPAc (28 kg). The aqueous layer was extracted with IPAc (28.8 kg) and the combined organic layers were washed with water (30 kg), then concentrated to ca. 24 kg at 40° C. under vacuum. Me-cyclohexane (19 kg) was added at 20° C. and the mixture was concentrated to ca. 24 kg at 40° C. under vacuum. This step was repeated twice more. The concentrate was diluted with Me-cyclohexane (29 kg) and stirred for 1 hr at 15-25° C. The mixture was filtered, and the wet cake was rinsed with Me-Cyclohexane (59 kg). The wet cake was dried under vacuum at 35-45° C. for 16 hrs to give (3R)-6-hydroxy-3,4-dihydro-2H-1-benzopyran-3-carboxylic acid (3.02 kg, 50.2% yield).

Chiral purity for Compound P2 was determined by supercritical fluid chromatography (SFC):
Instrument: Waters Acquity UPCC with PDA detector
Column: Daicel IC 4.6×250 mm, 5.0 μm column, PN: 83325
Wavelength: 300 nm
Column Temperature: 30° C.
Sampler Temperature: 20° C.
Flow Rate: 1.5 mL/min
Injector Volume: 5 μL
Strong Wash Solvent: MeOH
Weak Wash Solvent: MeOH:IPA=1:1 (v/v)
Seal Wash: MeOH
ABPR Pressure: 2000 psi
Mobile Phase A: CO₂
Mobile Phase B: 0.1% DEA in EtOH (v/v)
Gradient program:

| Time | | |
|---|---|---|
| (min) | A % | B % |
| Initial | 80 | 20 |
| 4.00 | 75 | 25 |
| 6.00 | 60 | 40 |
| 9.00 | 60 | 40 |
| 9.10 | 80 | 20 |
| 14.00 | 80 | 20 |

Run Time: 14.0 min
Components: RT (RRT)
Compound P2 (R): 3.9 min (1.00)
Compound P1: 4.5 min (1.15)

### B. Synthesis of 5-fluoro-3,4-dihydro-1,8-naphthyridin-2 (1H)-one

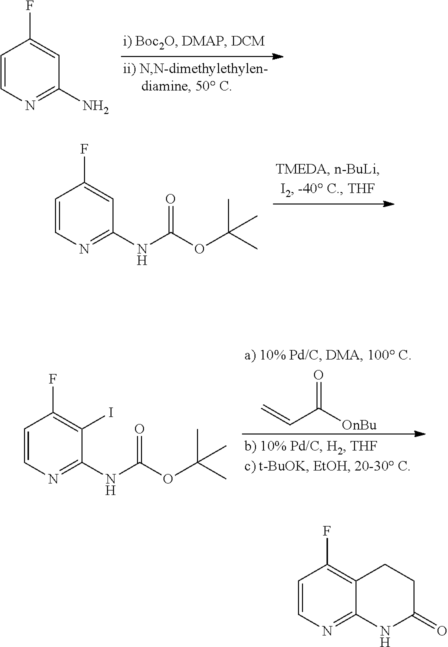

Step 1: To a stirred solution of 4-fluoro-2-pyridinamine (10.6 kg, 94.55 mol) in THF (104.0 kg) was added DMAP (0.59 kg, 4.82 mol), maintaining the temperature between 8-12° C. In a separate reaction vessel, Boc₂O (24.9 kg, 114.09 mol) was dissolved in THF (19 kg) with stirring, maintaining the temperature between 20-30° C. and stirred for 30 minutes. This solution was then slowly transferred into the vessel containing the 4-fluoro-2-pyridinamine at 10° C. and the mixture was stirred for 7 hours.

N′,N′-dimethylethane-1,2-diamine (10.05 kg, 114.01 mol) was then added to the reaction mixture slowly at 10° C. and the resulting mixture was stirred, maintaining the temperature between 38-42° C. for 22 hours. Water (42 kg) was then added over 2 hours at 25° C. the mixture was stirred at between 20-30° C. for 2 hours. Water (202 kg) was then added over 6 h maintaining the temperature at 25° C. and the mixture was stirred at between 20-30° C. for 1 hour. The vessel was then cooled to 10° C. over 2 hours and stirred for 5 hours. The mixture was filtered at 10° C. and the wet cake was washed with 38.6 kg of water/THF 1/3 (v/v). The wet cake was dried at 45-55° C. for 23 hours to give (4-fluoro-pyridin-2-yl)-carbamic acid tert-butyl ester (15.98 kg, 78.4% yield). LCMS (ES+): 16.59 min, m/z 156.97 [M-tBu]+.

Step 2: Solutions of (4-fluoro-pyridin-2-yl)-carbamic acid tert-butyl ester (12.6 kg, 59.36 mol) and TMEDA (17.78 kg, 153.0 mol) in THF (130 kg, 12 vol.) at 111.4 mL min-1 and n-BuLi (1.6 M in n-hexane)(45.25 kg, 168.8 mol) at 40 mL min-1 were each fed into a flow reactor at-40° C. Residency time in this flow reactor was 14 min before the solution entered another flow reactor at-55 to −40° C. Simultaneously, I₂ (26.7 kg, 95.3 mol) in THF (105.3 kg) was fed into this flow reactor at 70 mL min⁻¹. Residency time for the iodination was 14 min at −55 to −40° C. before being adjusted to 0-10° C. and being quenched with a feed of 5.0 eq. AcOH in water, for 10 min before being transferred to a separation vessel.

The organic layer was separated and treated with 2.0 eq. of Na₂S₂O₃ (16.7% in water), and the organic layer was separated and diluted with EtOAc (88.2 L) and water (37.8 L). The organics were collected and washed with water (3×38.2 kg) and concentrated in vacuo below 30° C. to 50 L. IPAc (58 kg) was added and the resulting mixture concentrated in vacuo to around 4 vol. This process was repeated to remove residual THF to below 1% and the resulting mixture was stirred at 10 to 25° C. for 3 h, filtered and the filter cake was washed with IPAc (37 kg). The wet cake was dried at 30-40° C. in vacuo to give the product (4-fluoro-3-iodo-pyridin-2-yl)-carbamic acid tert-butyl ester (15.1 kg, 75.2% yield). LCMS (Method A, ES+): 14.49 min, m/z 282.73 [M-tBu]+.

Step 3a: N,N-Dimethylacetamide (132 kg) was mechanically stirred and N2 bubbled through the reaction vessel for 12 hours. Et3N (10.8 kg, 106.73 mol), butyl prop-2-enoate (10.4 kg, 81.149 mol), (4-fluoro-3-iodo-pyridin-2-yl)-carbamic acid tert-butyl ester (14.4 kg, 42.59 mol), and 10% wet Pd/C (1.45 kg) were added and the reaction vessel atmosphere was evacuated and replaced with N₂ three times. Under N2, the mixture was heated to between 95-105° C. and stirred for 16 h. The mixture was then cooled and filtered through Celite® (19.95 kg), washing through with EtOAc (63.6 kg).

The filtrate was diluted with EtOAc (33 kg) and water (106 kg) and the mixture was stirred for 2 h, stood for 2 h and then the layers separated. The aqueous layer was extracted with 3×65 kg of EtOAc, with 1 hour of stirring and 2 hours of standing at 20-30° C. for each extraction. The combined organics were washed with 3×71 kg of water at 20-30° C., with 1 hour of stirring and 2 hours of standing at 20-30° C. for each wash. The organic layer was concentrated to 30-45 kg, diluted with THF (75 kg) and then THF (80 kg) added and the solution concentrated to around one-sixth volume. This was repeated 3 further times to reduce the EtOAc content to around 1%. This gave butyl (2E)-3-(2-amino-4-fluoropyridin-3-yl) prop-2-enoate as a solution in THF (50.4 kg total, 8.52 kg, 84% yield of product). LCMS (ES+): 17.69 min, m/z 239.08 [M+H]+.

Step 3b: Two identical reactions were performed. To a stirring solution of butyl (2E)-3-(2-amino-4-fluoropyridin-3-yl) prop-2-enoate (4.19 kg, 17.58 mol) in THF (20.61 kg) was added 10% wet Pd/C (0.80 kg). The reaction atmosphere was evacuated and replaced with Argon three times, and then evacuated and replaced with H₂ three times. The H2 pressure was adjusted to between 30-40 psi and the reaction was heated to between 35-45° C., stirring for 18 h. The mixture was filtered though Celite® (8.2 kg) washing through with THF (21 kg) to give butyl 3-(2-amino-4-fluoropyridin-3-yl) propanoate as a solution in THF.

Step 3c: The two butyl 3-(2-amino-4-fluoropyridin-3-yl) propanoate solutions in THF were combined and concentrated to around one-fifth volume. EtOH (51 Kg) was added and the resulting solution concentrated to around one-fifth volume. This process was repeated a further 4 times to reduce residual THF to around 0.5%. EtOH (11 kg) and t-BuOK (0.20 kg, 1.8 mol) were added, before stirring at 35° C. for 8 h. The mixture was neutralised with 1M HCl (1.6 kg) at 25° C. and diluted with water (42 kg). The mixture was cooled to between 5-15° C. and stirred for 3 h. The precipitate was filtered, and the filter cake washed with 2×27 kg of 1/3 (v/v) EtOH/water. The wet cake was dried in vacuo for 24 h at 40-50° C. to give 5-fluoro-1,2,3,4-tetrahydro-1,8-naphthyridin-2-one (4.9 kg, 79% yield over 2 steps). LCMS (ES+): 7.83 min, m/z 166.99 [M+H]+.

### C. Synthesis of Compounds A-1

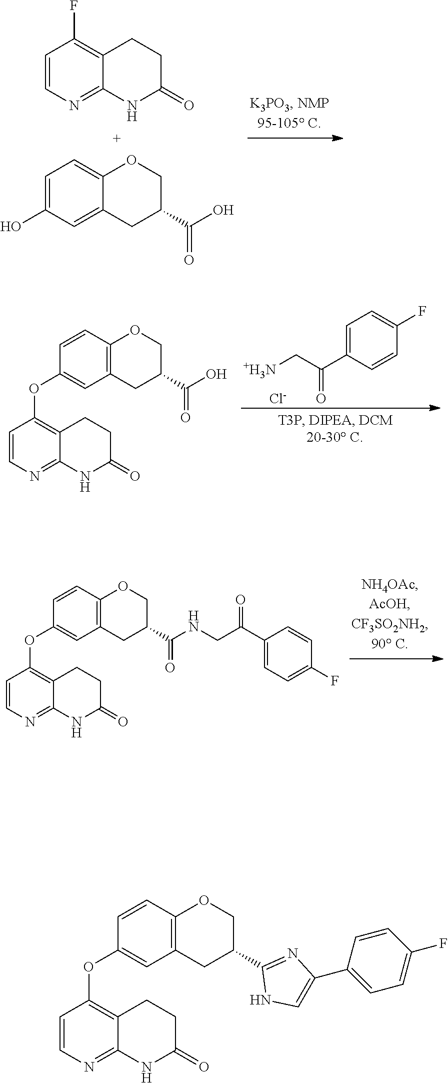

Step 1: To a stirred suspension of (3R)-6-hydroxy-3,4-dihydro-2H-1-benzopyran-3-carboxylic acid (1.73 kg, 8.91 mol, 98.9% chiral purity) in N₂-degassed NMP (54 kg) was added 5-fluoro-1,2,3,4-tetrahydro-1,8-naphthyridin-2-one (1.54 kg, 9.27 mol) and K₃PO₄ (7.7 kg, 36.27 mol) and the reaction mixture was stirred at 95-105° C. for 24 hrs.

The reaction was then cooled to 20-30° C. and diluted with THF (15.8 kg) and then stirred for 4 hrs at −15 to −5° C. The mixture was filtered and the filter cake washed with THF (19.8 kg). The wet cake was stirred in water (79 kg) for 2 hrs at 15-25° C., then taken to pH1 by drop-wise addition of 2 N HCl (40 kg). The resultant suspension was stirred for 3 hrs at 15-25° C. and filtered and the filter cake washed with water (44 kg). The wet cake was dried at 50-60° C. under vacuum for 36 hrs, then at 55-65° C. for a further 30 hrs, to give (3R)-6-[(7-oxo-5,6,7,8-tetrahydro-1,8-naphthyridin-4-yl)oxy]-3,4-dihydro-2H-1-benzopyran-3-carboxylic acid (2.80 kg, 87.5% yield, 99.2% chiral purity). LCMS (ES+): 8.79 min, m/z 341.08 [M+H]+.

Chiral purity for (3R)-6-[(7-oxo-5,6,7,8-tetrahydro-1,8-naphthyridin-4-yl)oxy]-3,4-dihydro-2H-1-benzopyran-3-carboxylic acid was determined by SFC:
Column: Daicel OD-3R 4.6×150 mm, 3.0 μm column, PN: 14824
Wavelength: 220 nm
Column Temperature: 40° C.
Sampler Temperature: 20° C.
Flow Rate: 1.5 mL/min
Injector Volume: 5 μL
Strong Wash Solvent: MeOH
Weak Wash Solvent: MeOH:IPA=1:1 (v/v)
Seal Wash: MeOH
ABPR Pressure: 2000 psi
Mobile Phase A: CO₂
Mobile Phase B: 0.1% TFA in MeOH (v/v)
Gradient program:

| Time | | |
|---|---|---|
| (min) | A % | B % |
| Initial | 80 | 20 |
| 4.00 | 65 | 35 |
| 7.00 | 60 | 40 |
| 9.00 | 60 | 40 |
| 9.10 | 80 | 20 |
| 12.00 | 80 | 20 |

Run Time: 12.0 min

Step 2: To a stirring mixture of (3R)-6-[(7-oxo-5,6,7,8-tetrahydro-1,8-naphthyridin-4-yl)oxy]-3,4-dihydro-2H-1-benzopyran-3-carboxylic acid (2.758 kg, 8.10 mol, 99.2% chiral purity) in N₂-degassed DCM (73 kg) was added 2-(4-fluorophenyl)-2-oxoethan-1-aminium chloride (2.32 kg, 12.24 mol) and T3P (8.50 kg, 13.36 mol), rinsing into the reaction mixture with DCM (10 kg). DIPEA (5.80 kg, 44.88 mol) was added dropwise across 3 hours and the reaction was stirred for 8 hrs at 20-30° C.

The reaction was then diluted with MTBE (42 kg) and concentrated to 38 L under vacuum at no more than 40° C. The concentrate was diluted with MTBE (16 kg) and DCM (7.5 kg) and then reconcentrated to 41 L under vacuum at no more than 40° C. The concentrate was stirred for 1.5 hrs at 15-25° C. and filtered, washing the wet cake with 12 kg of MTBE/DCM (2/1, v/v). The wet cake was resuspended in 38 kg of MTBE/DCM (2/1, v/v) and stirred for 7 hrs at 15-25° C. The mixture was then filtered and the filter cake washed with 13 kg of MTBE/DCM (2/1, v/v). The wet cake was then dried under vacuum at 55-65° C. for 24 hrs to give (3R)—N-[2-(4-fluorophenyl)-2-oxoethyl]-6-[(7-oxo-5,6,7,8-tetrahydro-1,8-naphthyridin-4-yl)oxy]-3,4-dihydro-2H-1-benzopyran-3-carboxamide (3.40 kg, 87.1%, 99.1% chiral purity). LCMS (ES+): 15.01 min, m/z 476.01 [M+H]+.

Chiral purity for (3R)—N-[2-(4-fluorophenyl)-2-oxoethyl]-6-[(7-oxo-5,6,7,8-tetrahydro-1,8-naphthyridin-4-yl)oxy]-3,4-dihydro-2H-1-benzopyran-3-carboxamide was determined by SFC:
Instrument: Waters Acquity UPCC with PDA detector
Column: Daicel IH-3 4.6×150 mm, 3.0 μm column, PN: 89524
Wavelength: 220 nm
Reference wavelength: Off (This parameter is only applicable to Agilent and
Thermo instruments)
Column Temperature: 40° C.
Sampler Temperature: 20° C.
Flow Rate: 1.5 mL/min
Injector Volume: 5 μL
Strong Wash Solvent: MeOH
   MeOH:IPA=1:1 (v/v)
Weak Wash Solvent: For example, accurately transfer 500 mL IPA to 500 mL MeOH, mix well and degas by ultrasonic.
Seal Wash: MeOH
ABPR Pressure: 2000 psi
Mobile Phase A: CO₂
Mobile Phase B: MeOH
Gradient program:

| Time | | |
|---|---|---|
| (min) | A % | B % |
| Initial | 90 | 10 |
| 12.00 | 50 | 50 |
| 18.50 | 50 | 50 |
| 18.60 | 90 | 10 |
| 22.00 | 90 | 10 |

Components: RT
Desired enantiomer (R) 15.1 min (1.00)
Opposite enantiomer 16.1 min (1.07)

Step 3: CF₃SO₂NH₂ (1570 g, 25 eq.) was added to a solution of AcOH (1900 g, 9.5 vol.) at 40° C. over 30 minutes under a nitrogen atmosphere. NH₄OAc (811 g, 25 eq.) was then added to the reaction vessel at 35-40° C. over 1 hour under a nitrogen atmosphere. P2O₅ (106 g, 1.78 eq.) was then added to the reaction vessel at 35-40° C. over 30 minutes under a nitrogen atmosphere followed by further AcOH (150 g, 0.75 vol.). The mixture was then stirred for 2 hours at 35-40° C.

P2O₅ (13.5 g, 0.23 eq.) was then added to the mixture under a nitrogen atmosphere followed by AcOH (50 g, 0.25 vol.) under a nitrogen atmosphere. The mixture was then stirred for 18 hours at 35-40° C.

(3R)—N-[2-(4-fluorophenyl)-2-oxoethyl]-6-[(7-oxo-5,6,7,8-tetrahydro-1,8-naphthyridin-4-yl)oxy]-3,4-dihydro-2H-1-benzopyran-3-carboxamide (200.05 g, 1 eq.) was then added to the reaction mixture at 35-40° C. over 30 minutes under a nitrogen atmosphere. The reaction temperature was increased to 90-95° C. and stirred for 24 hours under a nitrogen atmosphere before the temperature was reduced to 40-50° C. NH₄OAc (486.5 g, 15 eq.) was added to the reaction mixture under a nitrogen atmosphere and the reaction temperature was increased to 90-95° C. and stirred for 24 hours.

The temperature was again reduced to 40-50° C. NH₄OAc (486.5 g, 15 eq.) was added to the reaction mixture under a nitrogen atmosphere and the reaction temperature was increased to 90-95° C. and stirred for 24 hours. After this time the temperature was again reduced to 40-50° C. NH₄₀Ac (486.5 g, 15 eq.) was added to the reaction mixture under a nitrogen atmosphere and the reaction temperature was increased to 90-95° C. and stirred for 24 hours.

The reaction temperature was then taken to 20-30° C. and aq. NaOH (50 vol, 5 wt. %) was charged to a separate reaction vessel and 0.7 g of 5-{[(3S)-3-[4-(4-fluorophenyl)-1H-imidazol-2-yl]-3,4-dihydro-2H-1-benzopyran-6-yl]oxy}-1,2,3,4-tetrahydro-1,8-naphthyridin-2-one was added as a seed to the cooled reaction mixture. The reaction mixture was then slowly transferred to the vessel containing the NaOH solution and the resulting mixture stirred at 20-30° C. for 12 hours. The reaction mixture was then filtered and the filter cake washed with water (20 vol.).

The filter cake was then dissolved in TFA (0.25 vol.), water (12.5 vol.), MeCN (7.5 vol.) and THF (2.5 vol.) and the resulting solution purified by prep-HPLC using the following conditions:
Column: YMC Triart 250×50 mm, 7 μm
Mobile phase: A for H₂O (0.1% TFA) and B for MeCN
Flow rate: 80 mL/min
Column temperature: room temperature
Wavelength: 220 nm, 254 nm
Cycle time: ˜ 31 min
Injection: 40 mL per injection

NH₃·H₂O was added to the combined fractions, causing a solid to crash out. The resulting mixture was filtered and the filtrate concentrated in vacuo to give 5-{[(3S)-3-[4-(4-fluorophenyl)-1H-imidazol-2-yl]-3,4-dihydro-2H-1-benzopyran-6-yl]oxy}-1,2,3,4-tetrahydro-1,8-naphthyridin-2-one (146.4 g, 75% yield, 98.6% chiral purity) as an off-white solid. LCMS (ES+): 23.00 min, m/z 457.40 [M+H]+.

Chiral purity for 5-{[(3S)-3-[4-(4-fluorophenyl)-1H-imidazol-2-yl]-3,4-dihydro-2H-1-benzopyran-6-yl]oxy}-1,2,3,4-tetrahydro-1,8-naphthyridin-2-one was determined by SFC:
Instrument: Waters Acquity UPCC with PDA detector or equivalent
Column: Daicel Chiralpak AS-3, 4.6×150 mm, 3.0 μm column, PN: 20524
Wavelength: 220 nm
Reference wavelength: instruments)
Off (This parameter is only applicable to Agilent and Thermo
Data mode: Absorbance-Compensated
Sampling Rate: 5 points/see
Column Temperature: 40° C.
Sampler Temperature: 20° C.
Flow Rate: 1.5 mL/min
Injector Volume: 5 μL
Strong Wash Solvent: MeOH
Weak Wash Solvent: MeOH:IPA=1:1 (v/v)
Seal Wash: MeOH
ABPR Pressure: 2000 psi
Mobile Phase A: CO₂
Mobile Phase B: 0.2% DEA in EtOH, v/v
Gradient program:

| Time | | |
|---|---|---|
| (min) | A % | B % |
| 0.00 | 55 | 45 |
| 10.00 | 55 | 45 |
| 10.10 | 50 | 50 |
| 20.00 | 50 | 50 |
| 20.10 | 55 | 45 |
| 23.00 | 55 | 45 |

Components: RT
Desired (S) enantiomer 6.7 min (1.00)
(R) enantiomer 8.2 min (1.22)

LCMS method and parameters for 5-{[(3S)-3-[4-(4-fluorophenyl)-1H-imidazol-2-yl]-3,4-dihydro-2H-1-benzopyran-6-yl]oxy}-1,2,3,4-tetrahydro-1,8-naphthyridin-2-one:
Instrument: Agilent 1260 HPLC with MS detector
Column: Waters Xbridge C18 4.6×150 mm, 3.5 μm, PN: 186003034
Wavelength: 210 nm
Column Temperature: 50° C.
Sampler Temperature: 20° C.
Flow Rate: 1.0 mL/min
Injector Volume: 5 μL
Needle Wash: ACN: Water=10:90 (v/v)
Mobile Phase A: 10 mM NH₄OAc in water
Mobile Phase B: ACN:MeOH-80:20, v/v
Gradient Program:

| Time | | |
|---|---|---|
| (min) | A % | B % |
| Initial | 95 | 5 |
| 17.00 | 60 | 40 |
| 24.00 | 45 | 55 |
| 27.0 | 5 | 95 |
| 30.0 | 5 | 95 |
| 30.10 | 95 | 5 |
| 34.0 | 95 | 5 |

Data Acquisition Time: 34 min
MS parameters

| General | Ion source | ESI | |
|---|---|---|---|
| MSD signal | Mode | SCAN | |
| setting | Polarity | positive and negative | |
| | lon Range | m/z = 50~m/z = 800 | |
| | Fragment | 70 | eV |
| MSD spray | Drying gas flow | 12.0 | L/min |
| chamber | Nebulizer pressure | 35 | psig |
| | Drying gas temperature | 350° | C. |
| | Capillary voltage | 3000 | V |

### Example 5. Single Crystal Analysis of (3R)-6-hydroxy-3,4-dihydro-2H-1-benzopyran-3-carboxylic acid (P2)

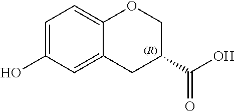

Compound P2 with 90% ee was used for single crystal cultivation. Single crystal growth experiments were conducted by using a variety of solvents through slow evaporation, vapor diffusion and slow cooling method. Single crystals suitable for structure analysis were obtained when slow evaporating in acetonitrile or tetrahydrofuran (THF)/water solvent system. Crystal structure was determined with the obtained single crystals in both acetonitrile and tetrahydrofuran/water solvent system.

Slow evaporating in acetonitrile: Approximate 5-10 mg of Compound P2 was added into a 40 mL glass vial with 10 mL of acetonitrile. After sonication for about 30 sec, the vial was centrifuged, then the solvent was evaporated under ambient condition.

Slow evaporating in tetrahydrofuran (THF)/water (v:v=2:1) solvent system: Approximate 5-10 mg of Compound P2 was added into a 1 mL glass vial with 0.4 mL of THF/water (v:v=2:1) solvent. After sonication for about 30 sec, obtained solutions or suspensions were filtrated by 0.45 μm membrane filter. The filtrates were transferred to a 1 mL glass vial. Then the vial was covered with a plastic lid with pin holes. The vial was placed in a fume hood to slow evaporate under ambient condition.

The single crystal structure of Compound P2 was determined at 170 (2) K. The absolute configuration of chiral C atom is determined to be “R” for single crystals obtained from both solvent systems. The crystals on the bottle vial along with single crystal were also collected for chiral purity test during slow evaporation in acetonitrile. The sample is in 97% chiral purity. And the retention time of the main peak is in accordance with that of the desired enantiomer, which means the absolute configuration of the Compound P2's desired enantiomer is R.

### Single Crystal X-Ray Diffractometer

| | |
|---|---|
| Instrument | Bruker D8 Venture |

Method

| Detector | CMOS area detector | |
|---|---|---|
| Temperature | 170(2) | K. |
| Radiation | Cu/K-Alpha1 | |
| | (λ = 1.5418 | Å) |
| X-ray generator power | 50 | kV, |
| | 10 | mA |
| Distance from sample | 40 | mm |
| to area detector | | |
| Exposure time | 2 | second |
| Resolution | 0.81 | Å |

Stereo microscope

| | |
|---|---|
| Instrument | OLYMPUS SZ2-ILST |

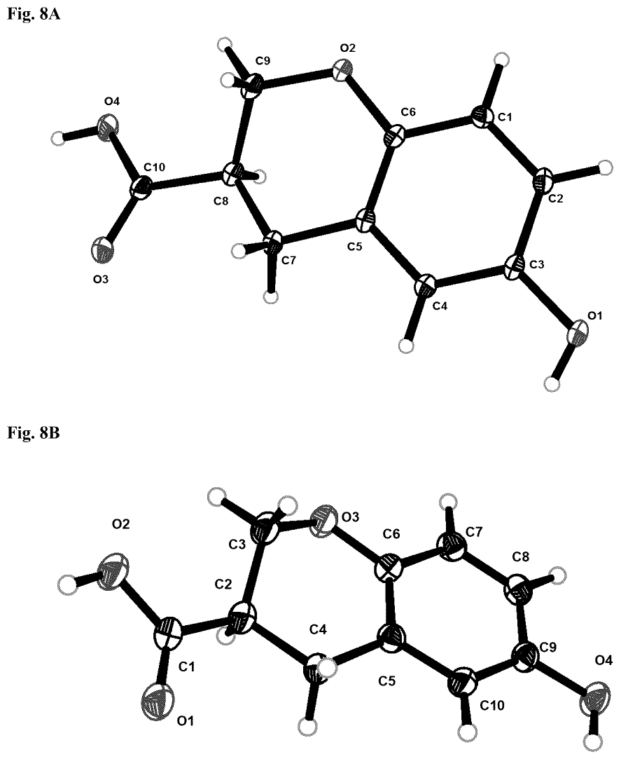

The crystalline form obtained from acetonitrile is crystallized in monoclinic system, P21 space group with Rᵢₙₜ=3.4%, absolute structure parameter=0.05 and the final R1=[I>2σ(I)]=3.6% at 170(2) K (Table 33A). No solvent molecule was contained in the asymmetric unit. The Ortep image of the single crystal of Compound P2 obtained from acetonitrile is shown in FIG. 8A.

TABLE 33A
Crystal data for crystalline form obtained from acetonitrile

| 2(C₁₀H₁₀O₄) | | F(000) = | 408 |
|---|---|---|---|
| Mᵣ = | 388.36 | Dₓ = | 1.464 Mg m⁻³ |
| Monoclinic, P2₁ | | Cu Kα radiation, | |
| | | λ = | 1.54178 Å |
| a = | 9.1688 (4) Å | Cell parameters | |
| | | from 5742 reflections | |
| b = | 5.6181 (2) Å | θ = | 2.6-72.3° |
| c = | 17.1506 (7) Å | μ = | 0.96 mm⁻¹ |
| β = | 94.172 (2)° | T = | 170 K. |

| | | |
|---|---|---|
| V = | 881.11 (6) Å³ | Block, colourless |
| Z = | 2 | 0.15 × 0.08 × 0.05 mm |

The crystalline form obtained from THF/water solvent system is crystallized in monoclinic system, P2₁ space group with Rᵢₙₜ=4.9%, absolute structure parameter=−0.04 and the final R1=[I>2σ(I)]=3.9% at 170 (2) K (Table 33B). No solvent molecule was contained in the asymmetric unit. The Ortep image of the single crystal of Compound P2 obtained from THF/water solvent system is shown in FIG. 8B.

TABLE 33B
Crystal data for crystalline form obtained from THF/water

| C₁₀H₁₀O₄ | | F(000) = | 408 |
|---|---|---|---|
| Mᵣ = | 194.18 | Dₓ = | 1.463 Mg m⁻³ |
| Monoclinic, P2₁ | | Cu Kα radiation, | |
| | | λ = | 1.54178 Å |
| a = | 9.1789 (6) Å | Cell parameters | |
| | | from 8617 reflections | |
| b = | 5.6108 (3) Å | θ = | 2.6-74.4º |
| c = | 17.1624 (8) Å | μ = | 0.96 mm⁻¹ |
| β = | 94.259 (4)° | T = | 170 K. |

| | | |
|---|---|---|
| V = | 881.44 (9) Å³ | Block, colourless |
| Z = | 4 | 0.15 × 0.08 × 0.05 mm |

### Example 6. Alternate Synthesis of 5-fluoro-3,4-dihydro-1,8-naphthyridin-2(1H)-one

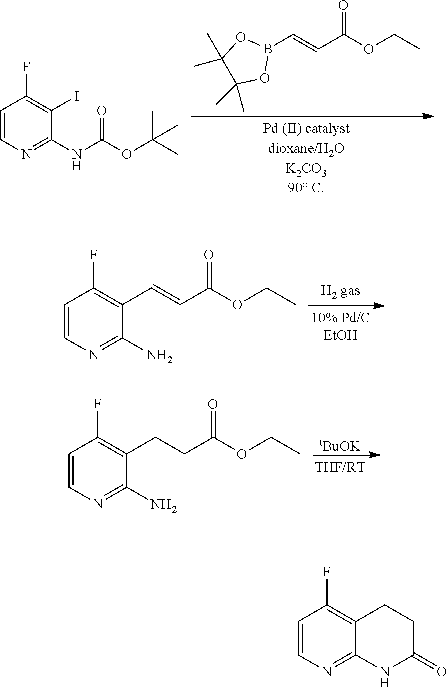

Step 1: tert-butyl N-(4-fluoro-3-iodo-2-pyridyl) carbamate (6.4 g, 18.9 mmol), K₂CO₃ (7.9 g, 57 mmol) and [(E)-2-(ethoxycarbonyl) vinyl] boronic acid-pinacol ester (4.92 g, 21.8 mmol) were taken up in 1,4-dioxane (120 mL) and water (25 mL) and then degassed for 15 minutes. To this mixture was then added [1,1′-Bis (diphenylphosphino)ferrocene] Palladium (II) chloride DCM complex (1.55 g, 1.9 mmol) and the reaction was then heated to 900C overnight. Initial 2-Boc position deprotection was observed first and proceeded cleanly; the Suzuki product conversion was effective after that. The reaction was evaporated to dryness and dissolved in DCM (150 mL) and treated with sat. aq. NH₄Cl solution (50 mL). Extracted with further DCM (2×150 mL), washed with brine, dried (MgSO4) and filtered before evaporating in vacuo to dryness. The residue was flash column chromatographed (silica 120 g) eluting with EtOAc in Pet. Ether (25 to 75%). Required compound eluted cleanly at ˜60% EtOAc in Pet. Ether to afford ethyl (E)-3-(2-amino-4-fluoro-3-pyridyl) prop-2-enoate (3.10 g, 14.8 mmol, 78% yield) as a waxy yellow solid. 1H NMR (400 MHz, DMSO-d₆), δ/ppm: 7.98 (dd, J=8.9, 5.6 Hz, 1H), 7.57 (d, J=16.1 Hz, 1H), 6.72 (s, 2H), 6.56-6.48 (m, 1H), 6.45 (dd, J=16.2, 1.2 Hz, 1H), 4.19 (q, J=7.1 Hz, 2H), 1.26 (t, J=7.1 Hz, 3H). UPLC-MS (ES+, Short acidic): 1.1 min, m/z 211.1 [M+H]+ (100%).

Step 2: Ethyl-(E)-3-(2-amino-4-fluoro-3-pyridyl) prop-2-enoate (1.0 g, 4.8 mmol) was taken up in EtOH (10 mL) and purged well with nitrogen. Palladium (10 wt. % on carbon powder, 50% wet) (225 mg, 0.21 mmol) was added and the reaction was subjected to an atmosphere of hydrogen gas and stirred overnight at room temperature. The reaction looked like predominantly the reduced side chain (˜90%) and the appearance of the required final cyclized hinge material (8%). The reaction was filtered to remove the Pd catalyst and evaporated to dryness to afford a crude mixture containing required product-ethyl 3-(2-amino-4-fluoro-3-pyridyl) propanoate (900 mg, 4.09 mmol, 86% yield) and 5-fluoro-3,4-dihydro-1H-1,8-naphthyridin-2-one (64 mg, 0.46 mmol, 10% yield) as components. 1H NMR (400 MHz, DMSO-d₆) δ/ppm: 7.79 (dd, J=9.1, 5.6 Hz, 1H), 6.38 (dd, J=9.2, 5.7 Hz, 1H), 6.11 (s, 2H), 4.04 (q, J=7.1 Hz, 2H), 2.73 (ddd, J=8.1, 6.8, 1.3 Hz, 2H), 2.45 (dd, J=8.4, 7.0 Hz, 2H), 1.16 (t, J=7.1 Hz, 3H).

Step 3: Ethyl-3-(2-amino-4-fluoro-3-pyridyl) propanoate (950 mg, 4.5 mmol) was taken up in THF (10 mL) and then treated with KOtBu (754 mg, 6.7 mmol) and stirred at room temperature for 30 mins. The reaction was quenched by the addition of sat. aq. NH₄Cl solution (2 mL), evaporated to dryness in vacuo and then taken up in water and sonicated well. The precipitate was slurried in water for 1 hr and the solid filtered, washed with water and dried in the vac oven to afford 5-fluoro-3,4-dihydro-1H-1,8-naphthyridin-2-one (691 mg, 4.2 mmol, 93% yield) as a fluffy white solid product. 1H NMR (400 MHz, DMSO-d₆) δ/ppm: 10.69 (s, 1H), 8.23-7.96 (m, 1H), 6.91 (dd, J=8.8, 5.7 Hz, 1H), 2.88 (dd, J=8.3, 7.1 Hz, 2H), 2.50 (s, 2H). UPLC-MS (ES+, Short acidic): 1.07 min, m/z 166.9 [M+H]+ (100%).

### Example 7. Alternate Synthesis of 5-fluoro-3,4-dihydro-1,8-naphthyridin-2(1H)-one

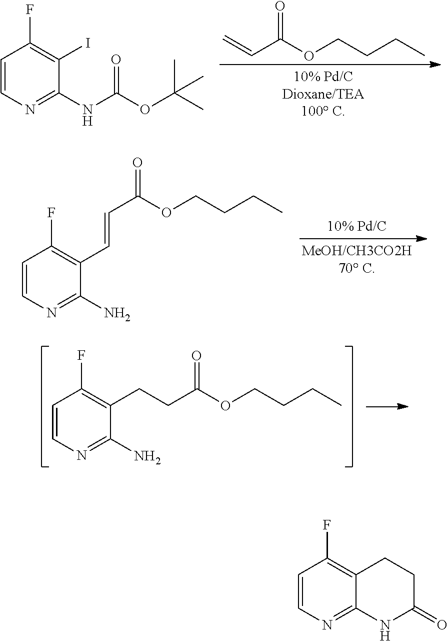

Step 1: tert-butyl N-(4-fluoro-3-iodo-2-pyridyl) carbamate (150 g, 444 mmol) was suspended in 1,4-dioxane (1.25 L) with butyl acrylate (159 mL, 1109 mmol) and TEA (155 mL, 1109 mmol) was added. Palladium (10 wt. % on carbon powder, 50% wet)(10.6 g, 99.8 mmol) was added and the reaction stirred and heated to reflux overnight and then cooled. UPLC-MS indicated 94% desired product. The reaction was diluted with water (750 mL) and EtOAc (500 mL) and filtered through celite to remove the catalyst. Washed through with EtOAc (500 mL). The layers were separated and the aqueous re-extracted with EtOAc (500 mL). The combined organic layers were washed with water (500 mL), dried (MgSO₄), filtered and reduced in-vacuo to afford butyl (E)-3-(2-amino-4-fluoro-3-pyridyl) prop-2-enoate (117.5 g. 439 mmol, 99% yield) as a yellow oil. ¹H NMR (400 MHz, DMSO-d₆) δ/ppm: 7.98 (dd, J=8.9, 5.5 Hz, 1H), 7.56 (d, J=16.1 Hz, 1H), 6.71 (s, 2H), 6 56-6 40 (m, 2H), 4.15 (t, J=6.6 Hz, 2H), 1.63 (dq, J=8 4, 6.7 Hz, 2H), 1.45-1.29 (m, 2H), 0.92 (t, J=7.3 Hz, 3H). UPLC-MS (ES⁺, Short acidic): 1.47 min, m/z 239.3 [M+H]⁺ (100%).

Step 2: Ethyl-(E)-3-(2-amino-4-fluoro-3-pyridyl) prop-2-enoate (1.0 g, 4.8 mmol) was taken up in EtOH (10 mL) and purged well with nitrogen. Palladium (10 wt. % on carbon powder, 50% wet)(225 mg, 0.21 mmol) was added and the reaction was subjected to an atmosphere of hydrogen gas and stirred overnight at room temperature. The reaction looked like predominantly the reduced side chain (˜90%) and the appearance of the required final cyclised material (8%). The reaction was filtered to remove the Pd catalyst and evaporated to dryness to afford a crude mixture containing required product-ethyl 3-(2-amino-4-fluoro-3-pyridyl) propanoate (900 mg, 4.09 mmol, 86% yield) and S-fluoro-3,4-dihydro-1H-1,8-naphthyridin-2-one (64 mg, 0.46 mmol, 10% yield) as components. ¹H NMR (400 MHz, DMSO-d₆) δ/ppm: 7.79 (dd, J=9.1, 5.6 Hz, 1H), 6.38 (dd, J=9.2, 5.7 Hz, 1H), 6.11 (s, 2H), 4.04 (q, J=7.1 Hz, 2H), 2.73 (ddd, J=8.1, 6.8, 1.3 Hz, 2H), 2.45 (dd, J=8.4, 7.0 Hz, 2H), 1.16 (t, J=7.1 Hz, 3H).

Step 3. Ethyl-3-(2-amino-4-fluoro-3-pyridyl) propanoate (950 mg, 4.5 mmol) was taken up in THF (10 mL) and then treated with KO^{t}Bu (754 mg, 6.7 mmol) and stirred at room temperature for 30 mins. The reaction was quenched by the addition of sat. aq. NH₄Cl solution (2 mL), evaporated to dryness in vacuo and then taken up in water and sonicated well. The precipitate was slurried in water for 1 hr and the solid filtered, washed with water and dried in the vac oven to afford 5-fluoro-3,4-dihydro-1H-1,8-naphthyridin-2-one (691 mg, 4.2 mmol, 93% yield) as a fluffy white solid product. ¹H NMR (400 MHz, DMSO-d₆) δ/ppm: 10.69 (s, 1H), 8.23-7.96 (m, 1H), 6.91 (dd, J=8.8, 5.7 Hz, 1H), 2.88 (dd, J=8.3, 7.1 Hz, 2H), 2.50 (s, 2H) UPLC-MS (ES+, Short acidic): 1.07 min, m/z 166.9 [M+H]⁺ (100%).

### Example 8. Biological Assays

HCT-116 AlphaLISA SureFire pERK1/2 Cellular Assay

The human HCT-116 colorectal carcinoma cell line (ATCC CCL-247) endogenously expresses the KRAS^{G13D} mutation, which leads to constitutive activation of the MAP kinase pathway and phosphorylation of ERK. To determine whether compounds inhibit constitutive ERK phosphorylation in HCT-116 cells, they were tested using AlphaLISA® SureFire® technology (Perkin Elmer p-ERK 1/2 p-T202/Y204 assay kit ALSU-PERK-A10K). Assay read outs took place 2 or 24 hours after dosing with compounds. On the first day, HCT-116 cells were harvested, resuspended in growth medium (McCoys5A with Glutamax (Life Technologies 36600021) and 10% heat-inactivated fetal bovine serum (Sigma F9665)), and counted. Cells were plated in 100 μl per well in each well of a 96-well culture dish (Sigma CLS3598) to a final density of 30,000 (2 hr read) or 15,000 (24 hr read) cells per well and incubated over night at 37° C. and 5% CO₂. On day 2, the growth medium was exchanged for dosing medium (McCoys5A with Glutamax (Life Technologies 36600021) and 1% heat-inactivated fetal bovine serum (Sigma F9665)) and the cells were dosed with compounds to produce a 10-point dose response, where the top concentration was 1 μM and subsequent concentrations were at ⅓ log dilution intervals. A matched DMSO control was included. The cells were subsequently incubated for either 2 or 24 hours at 37° C. and 5% CO₂. After incubation, media was removed and the cells were incubated with lysis buffer containing phosphatase inhibitors for 15 minutes at room temperature. Cell lysates were transferred to a ½ area 96 well white Optiplate™ (Perkin Elmer 6005569) and incubated with anti-mouse IgG acceptor beads, a biotinylated anti-ERK1/2 rabbit antibody recognizing both phosphorylated and non-phosphorylated ERK1/2, a mouse antibody targeted to the Thr202/Tyr204 epitope and recognizing phosphorylated ERK proteins only, and streptavidin-coated donor beads. The biotinylated antibody binds to the streptavidin-coated donor beads and the phopsho-ERK1/2 antibody binds to the acceptor beads. Plates were read on an EnVision reader (Perkin Elmer) and excitation of the beads at 680 nm with a laser induced the release of singlet oxygen molecules from the donor beads that trigger energy transfer to the acceptor beads in close proximity, producing a signal that can be measured at 570 nm. Both antibodies bound to phosphorylated ERK proteins, bringing the donor and acceptor beads into close proximity. All data were analyzed using the Dotmatics or GraphPad Prism software packages. Inhibition of ERK phosphorylation was assessed by determination of the absolute IC₅₀ value, which is defined as the concentration of compound required to decrease the level of phosphorylated ERK proteins by 50% when compared to DMSO control.

WiDr AlphaLISA SureFire pERK1/2 Cellular Assay

The human WiDr colorectal adenocarcinoma cell line (ATCC CCL-218) endogenously expresses the BRAF^{V600E} mutation, which leads to constitutive activation of the MAP kinase pathway and phosphorylation of ERK. To determine whether compounds inhibit constitutive ERK phosphorylation in WiDr cells, they were tested using AlphaLISA® SureFire® technology (Perkin Elmer p-ERK1/2 p-T202/Y204 assay kit ALSU-PERK-A10K). The main procedure is essentially the same as for HCT-116 cells (above), with the following adjustments to the growth medium (Eagle's Minimum Essential Medium (Sigma M2279) with 1× Glutamax (Life Technologies 35050038), 1× Sodium-Pyruvate (Sigma S8636), and 10% heat-inactivated fetal bovine serum (Sigma F9665)), the dosing medium (Eagle's Minimum Essential Medium (Sigma M2279) with 1× Glutamax (Life Technologies 35050038), 1× Sodium-Pyruvate (Sigma S8636), and 1% heat-inactivated fetal bovine serum (Sigma F9665)), and the seeding densities (2 hr: 50,000 cells per well; 24 hr: 35,000 cells per well). Moreover, the compounds were dosed in ½ log dilution intervals with the top concentration of 10 μM.

HCT-116 AlphaLISA SureFire pERK1/2 Cellular Assay (Dimer)

The human HCT-116 colorectal carcinoma cell line (ATCC CCL-247) endogenously expresses the KRAS^{G13D} mutation, which leads to constitutive activation of the MAP kinase pathway and phosphorylation of ERK. First generation RAF inhibitors can promote RAF dimer formation in KRAS mutant tumours leading to a paradoxical activation of the pathway. To determine whether compounds can circumvent this problem and inhibit RAF dimers in HCT-116 cells, they were tested using AlphaLISA® SureFire® technology (Perkin Elmer p-ERK 1/2 p-T202/Y204 assay kit ALSU-PERK-A10K). The main procedure is essentially the same as described above, with the following adjustments: Cells were seeded with the seeding density of 30,000 cells per well. On the second day (the day of dosing) no medium change was performed and the cells were dosed with 1 μM of Encorafenib for 1 hour (at 37° C. and 5% CO₂) to induce RAF dimers and promote paradoxical dimer-dependent pERK signalling. After incubation, the cells were washed, 100 μl fresh growth medium was added, and cells were dosed with compounds of interest to produce a 10-point dose response, where the top concentration was 10 μM and subsequent concentrations are at ½ log dilution intervals. Cells were incubated for another hour at 37° C. and 5% CO₂ before lysis and processing with the pERK AlphaLISA® SureFire® kit as described above.

A375 AlphaLISA SureFire pERK1/2 Cellular Assay (Monomer)

The human A375 melanoma cell line (ATCC CRL-1619) endogenously expresses the BRAF^{V600E} mutation, which leads to constitutive activation of the MAP kinase pathway and phosphorylation of ERK. In BRAF^{V600E} mutant tumours, BRAF signals as a monomer to activate ERK. To determine whether compounds can inhibit BRAF monomers in A375 cells, they were tested using AlphaLISA® SureFire® technology (Perkin Elmer p-ERK1/2 p-T202/Y204 assay kit ALSU-PERK-A10K). The main procedure is essentially the same as described above for HCT-116 cells, with the following adjustments: The A375 cells were cultivated and dosed in Dulbecco's modified Eagle's medium containing 4.5 g/L D-glucose (Sigma D6546), 10% heat-inactivated fetal bovine serum (Sigma F9665), and 1% Sodium-Pyruvate (Sigma S8636), and seeded with a seeding density of 30,000 cells per well. No media exchange was performed before dosing with compounds to produce a 10-point dose response, where the top concentration was 10 UM and subsequent concentrations were at ½ log dilution intervals. Subsequently, the cells were incubated for 1 hour at 37° C. and 5% CO₂ before lysis.

### HCT-116 CellTiter-Glo 3D) Cell Proliferation Assay

The human HCT-116 colorectal carcinoma cell line (ATCC CCL-247) endogenously expresses the KRASG13D mutation, which leads to enhanced survival and proliferative signaling. To determine whether compounds inhibit the proliferation of HCT-116 cells, they are tested using the CellTiter-Glo® 3D Cell Viability Assay Kit (Promega G9683). On the first day, HCT-116 cells were harvested, resuspended in growth medium (McCoys5A with Glutamax (Life Technologies 36600021) with 10% heat-inactivated fetal bovine serum (Sigma F9665)), and counted. Cells were plated in 100 μl per well in each well of a Corning 7007 96-well clear round bottom Ultra-Low Attachment plate (VWR 444-1020) to a final density of 1000 cells per well. Cells were seeded for pre- and post-treatment readouts. The cells were then incubated at 37° C. and 5% CO₂ for 3 days (72 hours) to allow spheroid formation. After 72 hours, the plate seeded for a pre-treatment read was removed from the incubator to allow equilibration to room temperature for 30 minutes, before CellTitre-Glo® reagent was added to each well. The plates were incubated at room temperature for 5 minutes shaking at 300 rpm, followed by an incubation of 25 minutes on the benchtop before being read on the Envision reader (Perkin Elmer) as described below. On the same day, the cells plated for the post-treatment readout were dosed with compounds to produce a 9-point dose response, where the top concentration was 15 M and following concentrations were at ½ log dilution intervals. These cells were subsequently incubated at 37° C. and 5% CO₂ for another 4 days (96 hours). After 4 days, the plate was removed from the incubator to allow equilibration to room temperature for 30 minutes and treated with CellTitre Glo® reagent as stated above. The method allows the quantification of ATP present in the wells, which is directly proportional to the amount of viable-hence metabolically active-cells in 3D cells cultures. The CellTitre Glo® reagent lyses the cells and contains luciferin and a luciferase (Ultra-Glo™ Recombinant Luciferase), which in the presence of ATP and oxygen can produce bioluminescence from luciferin. Therefore, plates were read on an EnVision reader (Perkin Elmer) and luminescence signals were recorded. Cell proliferation was determined on 4 days after dosing relative to the pre-treatment read. All data were analyzed using the Dotmatics or GraphPad Prism software packages. Inhibition of proliferation was assessed by determination of the GIso value, which was defined as the concentration of compound required to decrease the level of cell proliferation by 50% when compared to DMSO control.

### WiDr CellTiter-Glo 3D Cell Proliferation Assay

The human WiDr colorectal adenocarcinoma cell line (ATCC CCL-218) endogenously expresses the BRAF^{V600E} mutation, which leads to enhanced survival and proliferative signaling. To determine whether compounds inhibit the proliferation of WiDr cells, they were tested using the CellTiter-Glo® 3D Cell Viability Assay Kit (Promega G9683) as stated for HCT-116 cells, with the following adjustments to the growth medium: Eagle's Minimum Essential Medium (Sigma M2279) with 1× Glutamax (Life Technologies 35050038), 1× Sodium-Pyruvate (Sigma S8636) and 10% heat-inactivated fetal bovine serum (Sigma F9665).

TABLE 34A
Cellular Assay Results

| | PERK | PERK | PERK | pERK | PERK |
|---|---|---|---|---|---|
| | A375 | HCT116 | HCT116 | HCT116 | WiDr |
| Compd | mono (1 hr) | dimer (1 hr) | (2 hr) Abs | (24 hr) Abs | (2 hr) Abs |
| No. | plC50 | plC50 | plC50 | plC50 | plC50 |
| A-rac | 7.26 | 7.31 | 7.13 | 7.06 | 7.25 |
| A-1 | 7.51 | 7.39 | 7.31 | 7.02 | 7.34 |
| A-2 | 6.56 | 7.45 | 7.16 | 6.94 | 6.79 |
| B-rac | 7.31 | 7.55 | 7.72 | 7.62 | 7.35 |
| B-1 or | 7.51 | 7.76 | 7.75 | 7.87 | 7.55 |
| B-2 | | | | | |
| Faster | | | | | |
| eluting | | | | | |
| isomer) | | | | | |
| B-1 or | 6.47 | 7.24 | 6.98 | 7.12 | 6.67 |
| B-2 | | | | | |
| (Slower | | | | | |
| eluting | | | | | |
| isomer) | | | | | |

TABLE 34B
Cellular Assay Results

| Compd | 3D HCT116 | 3D WiDr |
|---|---|---|
| No. | pGI50 | pGI50 |
| A-2 | 6.58 | 6.27 |
| A-1 | 7.39 | 7.28 |

### Microsomal Stability Assay

The stability studies were performed manually using the substrate depletion approach. Test compounds were incubated at 37° C. with cryo-preserved mouse or human liver microsomes (Corning) at a protein concentration of 0.5 mg·mL⁻¹ and a final substrate concentration of 1 μM. Aliquots were removed from the incubation at defined timepoints and the reaction was terminated by adding to ice-cold organic solvent. Compound concentrations were determined by LC-MS/MS analysis. The natural log of the percentage of compound remaining was plotted against each time point and the slope determined. The half-life (t_{1/2}) and CLᵢₙₜ were calculated using Equations 1 and 2, respectively. Data analysis was performed using Excel (Microsoft, USA).

$${t}_{1 / 2} ( min ) = 0.693 / - slope \qquad ( 1 )$$

$${CL}_{int} ( μ L / min / mg ) = ( LN ( 2 ) / {t}_{1 / 2} ( min ) ) * 1000 / microsomal protein ( mg / mL ) \qquad ( 2 )$$

HLM (human liver microsomes) and MLM (mouse liver microsomes) stability assay results are described in Table 34C.

### Hepatocyte Stability Assay

Hepatocyte stability studies were performed manually using the substrate depletion approach. Compounds were incubated at 37° C. with cryo-preserved mouse (Bioreclamation) or human (Corning) hepatocytes at a cell density of 0.5×10⁶ cells/mL and a final compound concentration of 1 μM. Sampling was performed at defined timepoints and the reaction was terminated by adding to ice-cold organic solvent. Compound concentrations were determined by LC-MS/MS analysis. The natural log of the percentage of compound remaining was plotted against each time point and the slope determined. The half-life (t_{1/2}) and CLᵢₙₜ were calculated using Equations 1 and 3, respectively. Data analysis was performed using Excel (Microsoft, USA).

$${CL}_{int} ( μ L / min / {10}^{6} cells ) = ( LN ( 2 ) / {t}_{1 / 2} ( min ) ) * 1000 / cell density ( {10}^{6} cells / mL ) \qquad ( 3 )$$

HLH (human liver hepatocytes) and MLH (mouse liver heptaocytes) stability assay results are described in Table 34C.

TABLE 34C
Stability

| | HLM | MLM | HLH (CLint) | MLH (CLint) |
|---|---|---|---|---|
| | (CLint) | (CLint) | μL/min/10⁶ | μL/min/10⁶ |
| Compd No. | μL/min/mg | μL/min/mg | cells | cells |
| A-rac | 26.9 | 30.6 | 4.6 | 23.4 |
| A-1 | 21.7 | 16 | 26.8 | 11 |
| A-2 | 11.8 | 80.1 | 11.4 | 12.5 |
| B-rac | 60.1 | 58.8 | 11.5 | nd |
| B-1 or B-2 | 49.8 | 48.2 | 26.9 | 18.9 |
| (Faster eluting | | | | |
| isomer) | | | | |
| B-1 or B-2 | 29.8 | 62.4 | 33.9 | 18.8 |
| (Slower eluting | | | | |
| isomer) | | | | |

### Plasma Protein Binding Assay

The plasma protein binding was determined by the equilibrium dialysis method. A known concentration of compound (5 μM) in previously frozen human or mouse plasma (Sera Labs) was dialysed against phosphate buffer using a RED device (Life Technologies) for 4 hours at 37° C. The concentration of compound in the protein containing (PC) and protein free (PF) sides of the dialysis membrane were determined by LC-MS/MS and the % free compound was determined by equation 4. Data analysis was performed using Excel (Microsoft, USA).

$$\% free = ( 1 - ( ( PC - PF ) / PC ) ) \times 100 \qquad ( 4 )$$

hPPB (human plasma protein binding) and mPPB (mouse plasma protein binding) results are described in Table 34D.

### FeSSIF Solubility Assay

1 mL of fed state simulated intestinal fluid (FeSSIF), prepared using FaSSIF/FeSSIF/FaSSGF powder (Biorelevant.com) and pH 5 acetate buffer, was added to 1.0 mg of compound and then incubated for 24 h (Bioshake iQ, 650 rpm, 37° C.). Following filtration under positive pressure, the concentration of compound in solution was assessed by LC-UV in comparison to the response for a calibration standard of known concentration (250 μM). FeSSIF solubility results are described in Table 34D.

TABLE 34D
Plasma Protein Binding and Solubility

| | hPPB | mPPB | FESSIF |
|---|---|---|---|
| Compd No. | (% free) | (% free) | sol (mg/L) |
| A-rac | 0.5 | 1.8 | |
| A-1 | 0.5 | 0.8 | 9.9 |
| A-2 | 0.7 | 0.9 | 9.4 |
| B-rac | 0.7 | 0.7 | |
| B-1 or B-2 (Faster | 0.4 | 0.4 | 31.4 |
| eluting isomer) | | | |
| B-1 or B-2 (Slower | 0.5 | 0.8 | 19.2 |
| eluting isomer) | | | |

The publications discussed herein are provided solely for their disclosure prior to the filing date of the present application. Nothing herein is to be construed as an admission that the present invention is not entitled to antedate such publication by virtue of prior invention.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### Numbered Embodiments

Embodiment 1. A method of synthesizing a compound of formula (IIb) or a pharmaceutically acceptable salt or tautomer thereof,

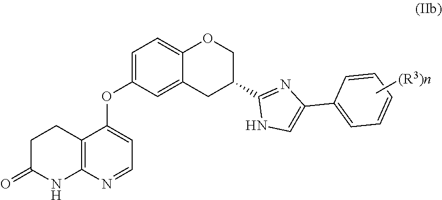

wherein:
R³ is halogen, —OR^{A}, —NR^{A}R^{B}, —SO₂RC, —SOR^{C}, —CN, C₁₋₄ alkyl, C₁₋₄ haloalkyl, or C₃₋₆ cycloalkyl, wherein the alkyl, haloalkyl and cycloalkyl groups are optionally substituted with 1 to 3 groups independently selected from: —OR^{A}, —CN, —SOR^{C}, or —NR^{A}R^{B},
R^{A} and R^{B} are each independently selected from H, C₁₋₄ alkyl and C₁₋₄ haloalkyl;
R^{C} is selected from C₁₋₄ alkyl and C₁₋₄ haloalkyl; and
n is 0, 1, 2, 3, or 4;
the method comprising:
a) reacting 5-fluoro-3,4-dihydro-1,8-naphthyridin-2 (1H)-one with (R)-6-hydroxychromane-3-carboxylic acid to provide (R)-6-((7-oxo-5,6,7,8-tetrahydro-1,8-naphthyridin-4-yl)oxy) chromane-3-carboxylic acid;

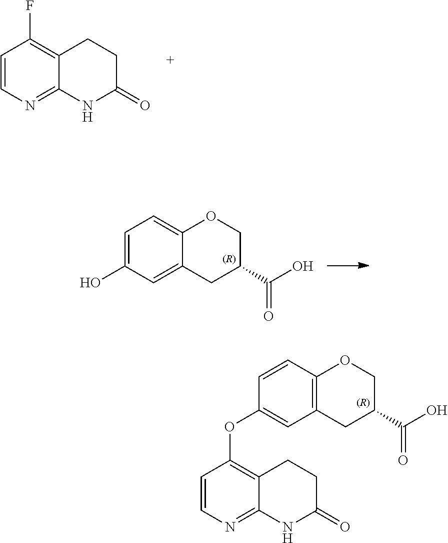

b) reacting (R)-6-((7-oxo-5,6,7,8-tetrahydro-1,8-naphthyridin-4-yl)oxy) chromane-3-carboxylic acid with a 2-amino-1-phenylethan-1-one, or a salt thereof, to provide a compound of formula 4B-(R),
wherein the 2-amino-1-phenylethan-1-one is optionally substituted with R³; and

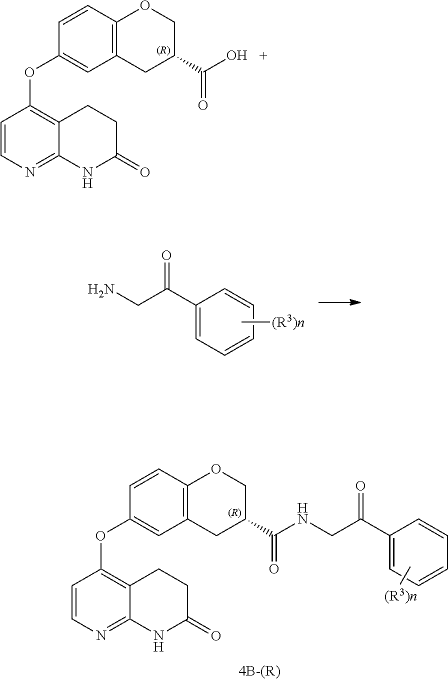

c) cyclizing the compound of formula 4B-(R) of step b) in the presence of ammonia or an ammonium salt to provide the compound of formula (IIb), or a pharmaceutically acceptable salt or tautomer thereof.

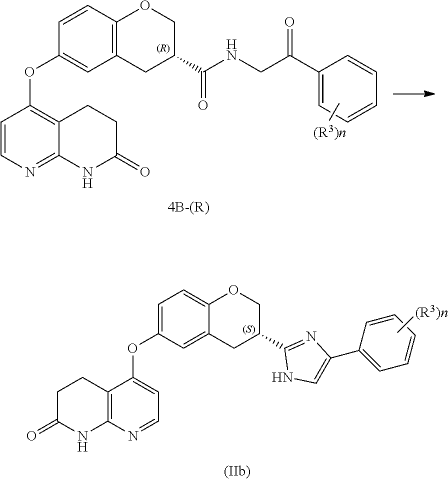

Embodiment 2. The method Embodiment 1, wherein (R)-6-hydroxychromane-3-carboxylic acid is prepared by chiral hydrogenation of 6-hydroxy-2H-chromene-3-carboxylic acid.

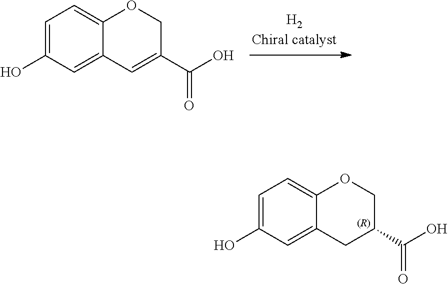

Embodiment 3. The method of Embodiment 2, wherein the chiral hydrogenation is performed in the presence of Ru or Rₕ catalyst and a chiral ligand.

Embodiment 4. The method of Embodiment 3, wherein the Ru or Rₕ catalyst is selected from Ru(OAc)₂, [RuCl₂(p-cym)]₂, Ru(COD)(Me-allyl)₂, Ru(COD)(TFA)₂, [Rh(COD)₂]OTf or [Rh(COD)₂]BF₄.

Embodiment 5. The method of Embodiment 3 or 4, wherein the Ru catalyst is selected from [RuCl₂(p-cym)]₂, Ru(COD)(Me-allyl)₂, or Ru(COD)(TFA)₂.

Embodiment 6. The method of any one of Embodiments 3-5, wherein the chiral ligand is selected from (R)-PhanePhos or (R)-An-PhanePhos.

Embodiment 7. The method of Embodiment 3, wherein the chiral hydrogenation is performed in the presence of a chiral Ru-complex or a chiral Rₕ-complex.

Embodiment 8. The method of Embodiment 7, wherein the chiral Ru-complex or the chiral Rₕ-complex is selected from [(R)-Phanephos-RuCl₂(p-cym)], or [(R)-An-Phanephos-RuCl₂(p-cym)].

Embodiment 9. The method of any one of Embodiments 2-8, wherein the chiral hydrogenation is performed with a substrate/catalyst loading in the range of about 25/1 to about 1,000/1.

Embodiment 10. The method of any one of Embodiments 2-8, wherein the chiral hydrogenation is performed with a substrate/catalyst loading in the range of about 200/1 to about 1,000/1.

Embodiment 11. The method of any one of Embodiments 2-10, wherein the chiral hydrogenation is performed in the presence of base.

Embodiment 12. The method of Embodiment 11, wherein the base is triethylamine, NaOMe or Na₂CO₃.

Embodiment 13. The method of Embodiment 11 or 12, wherein the base is used in about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 equivalent with respect to 6-hydroxy-2H-chromene-3-carboxylic acid.

Embodiment 14. The method of any one of Embodiments 2-13, wherein the chiral hydrogenation is performed at a temperature in the range of about 30° C. to about 50° C.

Embodiment 15. The method of any one of Embodiments 2-14, wherein the chiral hydrogenation is performed at a concentration of 6-hydroxy-2H-chromene-3-carboxylic acid in the range of about 0.2M to about 0.8M.

Embodiment 16. The method of any one of Embodiments 2-15, wherein the chiral hydrogenation is performed at hydrogen pressure in the range of about 2 bar to about 30 bar.

Embodiment 17. The method of any one of Embodiments 2-15, wherein the chiral hydrogenation is performed at hydrogen pressure in the range of about 3 bar to about 10 bar.

Embodiment 18. The method of any one of Embodiments 2-17, wherein the chiral hydrogenation is performed in an alcohol solvent.

Embodiment 19. The method of Embodiment 18, wherein the solvent is methanol, ethanol, or isopropanol.

Embodiment 20. The method of any one of Embodiments 1-19, wherein (R)-6-hydroxychromane-3-carboxylic acid has an enantiomeric excess of at least 90%.

Embodiment 21. The method of any one of Embodiments 1-19, wherein (R)-6-hydroxychromane-3-carboxylic acid has an enantiomeric excess of at least 95%.

Embodiment 22. The method of any one of Embodiments 1-21, wherein (R)-6-((7-oxo-5,6,7,8-tetrahydro-1,8-naphthyridin-4-yl)oxy) chromane-3-carboxylic acid has an enantiomeric excess of at least 90%.

Embodiment 23. The method of any one of Embodiments 1-21, wherein (R)-6-((7-oxo-5,6,7,8-tetrahydro-1,8-naphthyridin-4-yl)oxy) chromane-3-carboxylic acid has an enantiomeric excess of at least 95%.

Embodiment 24. The method of any one of Embodiments 1-23, wherein the compound of formula 4B-(R) of step b) has an enantiomeric excess of at least 90%.

Embodiment 25. The method of any one of Embodiments 1-23, wherein the compound of formula 4B-(R) of step b) has an enantiomeric excess of at least 95%.

Embodiment 26. The method of any one of Embodiments 1-25, wherein the compound of formula (IIb), or a pharmaceutically acceptable salt or tautomer thereof, has an enantiomeric excess of at least 90%.

Embodiment 27. The method of any one of Embodiments 1-25, wherein the compound of formula (IIb), or a pharmaceutically acceptable salt or tautomer thereof, has an enantiomeric excess of at least 95%.

Embodiment 28. The method of any one of Embodiments 1-25, wherein the compound of formula (IIb), or a pharmaceutically acceptable salt or tautomer thereof, has an enantiomeric excess of at least 98%.

Embodiment 29. The method of any one of Embodiments 1-28, wherein R³ is halogen, C₁₋₄ alkyl, —SO₂(C₁₋₄ alkyl).

Embodiment 30. The method of any one of Embodiments 1-28, wherein R³ is F, Cl, Br, or I.

Embodiment 31. The method of any one of Embodiments 1-30, wherein n is 0, 1, or 2.

Embodiment 32. The method of any one of Embodiments 1-31, wherein the compound is

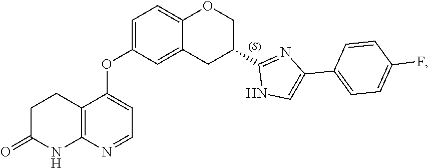

or a pharmaceutically acceptable salt or tautomer thereof.

Embodiment 33. A compound of formula (IIb), or a pharmaceutically acceptable salt or tautomer thereof, prepared by the method of any one of Embodiments 1-32.

Embodiment 34. A compound having the structure

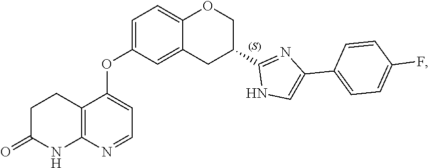

or a pharmaceutically acceptable salt or tautomer thereof, prepared by the method of any one of Embodiments 1-32.

Embodiment 35. The compound of Embodiments 33 or 34, wherein the compound has an enantiomeric excess of at least 90%.

Embodiment 36. The compound of any one of Embodiments 33-35, wherein the compound has an enantiomeric excess of at least 95%.

Embodiment 37. The compound of any one of Embodiments 33-36, wherein the compound has an enantiomeric excess of at least 98%.

Embodiment 38. The compound of any one of Embodiments 33-37, wherein the compound has a chemical purity of 85% or greater.

Embodiment 39. The compound of any one of Embodiments 33-38, wherein the compound has a chemical purity of 90% or greater.

Embodiment 40. The compound of any one of Embodiments 33-39, wherein the compound has a chemical purity of 95% or greater.

Embodiment 41. A pharmaceutical composition comprising a compound of any one of Embodiments 33-40 and a pharmaceutically acceptable excipient or carrier.

Embodiment 42. The pharmaceutical composition of Embodiment 41, further comprising an additional therapeutic agent.

Embodiment 43. The pharmaceutical composition of Embodiments 42, wherein the additional therapeutic agent is selected from an antiproliferative or an antineoplastic drug, a cytostatic agent, an anti-invasion agent, an inhibitor of growth factor function, an antiangiogenic agent, a steroid, a targeted therapy agent, or an immunotherapeutic agent.

Embodiment 44. A method of treating a condition which is modulated by a RAF kinase, comprising administering an effective amount of the compound of any one of Embodiments 33-40 to a subject in need thereof.

Embodiment 45. The method of Embodiment 44, wherein the condition treatable by the inhibition of one or more Raf kinases.

Embodiment 46. The method of Embodiment 44 or 45, wherein the condition is selected from cancer, sarcoma, melanoma, skin cancer, haematological tumors, lymphoma, carcinoma or leukemia.

Embodiment 47. The method of Embodiment 44 or 45, wherein the condition is selected from Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancer.

Embodiment 48. A method of treating cancer, comprising administering an effective amount of the compound of any one of Embodiments 33-40 to a subject in need thereof.

Embodiment 49. The method of Embodiment 48, wherein the cancer comprises at least one mutation of the BRAF kinase.

Embodiment 50. The method of Embodiment 49, wherein the cancer comprises a BRAF^{V600E} mutation.

Embodiment 51. The method of Embodiment 49, wherein the cancer is selected from melanomas, thyroid cancer, Barret's adenocarcinoma, biliary tract carcinomas, breast cancer, cervical cancer, cholangiocarcinoma, central nervous system tumors, glioblastomas, astrocytomas, ependymomas, colorectal cancer, large intestine colon cancer, gastric cancer, carcinoma of the head and neck, hematologic cancers, leukemia, acute lymphoblastic leukemia, myelodysplastic syndromes, chronic myelogenous leukemia, Hodgkin's lymphoma, non-Hodgkin's lymphoma, megakaryoblastic leukemia, multiple myeloma, hepatocellular carcinoma, lung cancer, ovarian cancer, pancreatic cancer, pituitary adenoma, prostate cancer, renal cancer, sarcoma, uveal melanoma or skin cancer.

Embodiment 52. The method of Embodiment 50, wherein the cancer is BRAF^{V600E} melanoma, BRAF^{V600E} colorectal cancer, BRAF^{V600E} papillary thyroid cancers, BRAF^{V600E} low grade serous ovarian cancers, BRAF^{V600E} glioma, BRAF^{V600E} hepatobiliary cancers, BRAF^{V600E} hairy cell leukemia, BRAF^{V600E} non-small cell cancer, or BRAF^{V600E} pilocytic astrocytoma.

Embodiment 53. The method of any one of Embodiment 2 46-52, wherein the cancer is colorectal cancer.

1-56. (canceled)

57. A method of synthesizing (R)-5-[3-[4-(4-fluorophenyl)-1H-imidazol-2-yl]chroman-6-yl]oxy-3,4-dihydro-1H-1,8-naphthyridin-2-one or (S)-5-[3-[4-(4-fluorophenyl)-1H-imidazol-2-yl]chroman-6-yl]oxy-3,4-dihydro-1H-1,8-naphthyridin-2-one having the structure: 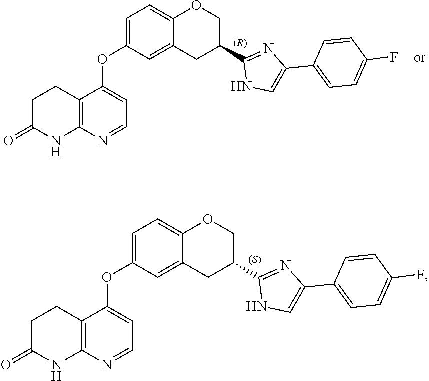
or a pharmaceutically acceptable salt or tautomer thereof, comprising:
a) reacting 6-hydroxy-2H-chromene-3-carboxylic acid with H₂ in the presence of a chiral catalyst to form (R)-6-hydroxychromane-3-carboxylic acid or (S)-6-hydroxychromane-3-carboxylic acid, wherein the chiral catalyst comprises a chiral ligand selected from (S)-PPhos, (R)-PPhos, (S)-Xyl-PPhos, (R)-Xyl-PPhos, (S)-PhanePhos, (R)-PhanePhos, (S)-Xyl-PhanePhos, (R)-Xyl-PhanePhos, (S,S)-Me-DuPhos, (R,R)-Me-DuPhos, (S,S)-iPr-DuPhos, (R,R)-iPr-DuPhos, (S,S)-NorPhos, (R,R)-NorPhos, (S,S)-BPPM, or (R,R)-BPPM, or Josiphos SL-J002-1; 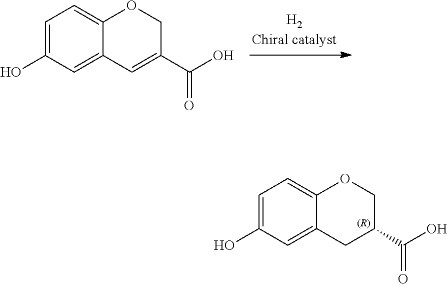
b) reacting 5-fluoro-3,4-dihydro-1,8-naphthyridin-2 (1H)-one with (R)-6-hydroxychromane-3-carboxylic acid or (S)-6-hydroxychromane-3-carboxylic acid prepared in step a) to provide (R)-6-((7-oxo-5,6,7,8-tetrahydro-1,8-naphthyridin-4-yl)oxy) chromane-3-carboxylic acid or (S)-6-((7-oxo-5,6,7,8-tetrahydro-1,8-naphthyridin-4-yl)oxy) chromane-3-carboxylic acid; 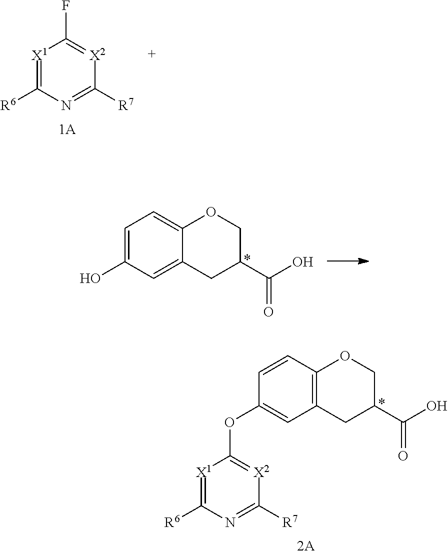
c) reacting (R)-6-((7-oxo-5,6,7,8-tetrahydro-1,8-naphthyridin-4-yl)oxy) chromane-3-carboxylic acid or (S)-6-((7-oxo-5,6,7,8-tetrahydro-1,8-naphthyridin-4-yl)oxy) chromane-3-carboxylic acid prepared in step b) with a 2-amino-1-(4-fluorophenyl) ethanone, or a salt thereof, to provide (R)—N-[2-(4-fluorophenyl)-2-oxo-ethyl]-6-[(7-oxo-6,8-dihydro-5H-1,8-naphthyridin-4-yl)oxy]chromane-3-carboxamide or (S)—N-[2-(4-fluorophenyl)-2-oxo-ethyl]-6-[(7-oxo-6,8-dihydro-5H-1,8-naphthyridin-4-yl)oxy]chromane-3-carboxamide; and 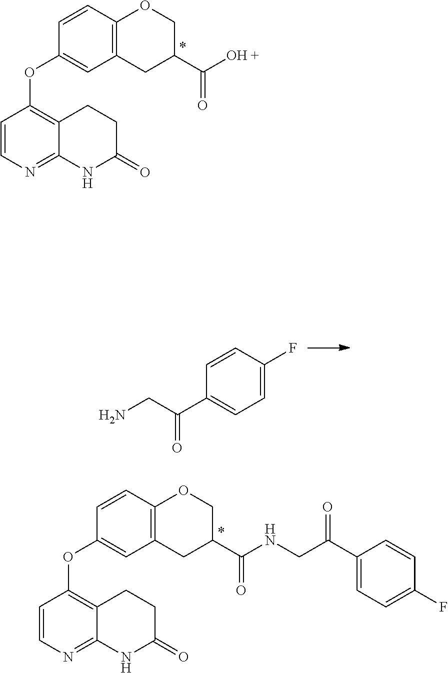
d) cyclizing (S)—N-[2-(4-fluorophenyl)-2-oxo-ethyl]-6-[(7-oxo-6,8-dihydro-5H-1,8-naphthyridin-4-yl)oxy]chromane-3-carboxamide or (R)—N-[2-(4-fluorophenyl)-2-oxo-ethyl]-6-[(7-oxo-6,8-dihydro-5H-1,8-naphthyridin-4-yl)oxy]chromane-3-carboxamide prepared by step c) in the presence of ammonia or an ammonium salt to provide (R)-5-[3-[4-(4-fluorophenyl)-1H-imidazol-2-yl]chroman-6-yl]oxy-3,4-dihydro-1H-1,8-naphthyridin-2-one or (S)-5-[3-[4-(4-fluorophenyl)-1H-imidazol-2-yl]chroman-6-yl]oxy-3,4-dihydro-1H-1,8-naphthyridin-2-one

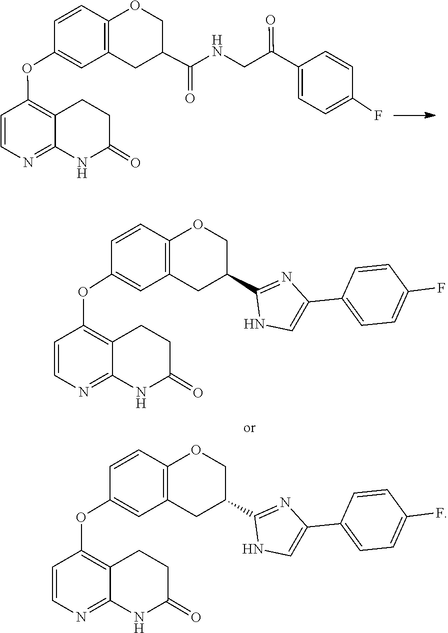 [SMILES: O=C1CCc2c(Oc3ccc4c(c3)CC(C(=O)NCC(=O)c3ccc(F)cc3)CO4)ccnc2N1 ; O=C1CCc2c(Oc3ccc4c(c3)C[C@H](c3nc(-c5ccc(F)cc5)c[nH]3)CO4)ccnc2N1 ; O=C1CCc2c(Oc3ccc4c(c3)C[C@@H](c3nc(-c5ccc(F)cc5)c[nH]3)CO4)ccnc2N1]

58. The method of claim 57, wherein the chiral catalyst in step a) comprises Ru or Rₕ.

59. The method of claim 57, wherein the chiral ligand is (S)-PhanePhos, (R)-PhanePhos, (S)-An-PhanePhos, or (R)-An-PhanePhos.

60. The method of claim 57, wherein the chiral catalyst comprising a chiral ligand in step a) is a chiral Ru-complex or a chiral Rₕ-complex selected from [(R)-Phanephos-RuCl₂(p-cym)], [(S)-Phanephos-RuCl₂(p-cym)], [(R)-An-Phanephos-RuCl₂(p-cym)], [(S)-An-Phanephos-RuCl₂(p-cym)], [(R)-Phanephos-Rh(COD)]BF₄, [(S)-Phanephos-Rh(COD)]BF₄, [(R)-Phanephos-Rh(COD)]OTf, or [(S)-Phanephos-Rh(COD)]OTf.

61. The method of claim 60, wherein the chiral Ru-complex or the chiral Rₕ-complex is [(R)-Phanephos-RuCl₂(p-cym)], [(S)-Phanephos-RuCl₂(p-cym)], [(R)-An-Phanephos-RuCl₂(p-cym)], or [(S)-An-Phanephos-RuCl₂(p-cym)].

62. The method of claim 57, wherein step a) is performed with a substrate/catalyst loading in the range of about 25/1 to about 1,000/1, wherein the substrate is 6-hydroxy-2H-chromene-3-carboxylic acid.

63. The method of claim 57, wherein step a) is performed with a substrate/catalyst loading in the range of about 200/1 to about 1,000/1, wherein the substrate is 6-hydroxy-2H-chromene-3-carboxylic acid.

64. The method of claim 57, wherein step a) is performed in the presence of a base.

65. The method of claim 64, wherein the base is triethylamine, NaOMe or Na₂CO₃.

66. The method of claim 64, wherein the base is used in about 2.0, about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 equivalent with respect to 6-hydroxy-2H-chromene-3-carboxylic acid.

67. The method of claim 57, wherein step a) is performed at a temperature in the range of about 30° C. to about 50° C.

68. The method of claim 57, wherein step a) is performed at a concentration of 6-hydroxy-2H-chromene-3-carboxylic acid in the range of about 0.2M to about 0.8M.

69. The method of claim 57, wherein step a) is performed at hydrogen pressure in the range of about 2 bar to about 30 bar.

70. The method of claim 57, wherein step a) is performed at hydrogen pressure in the range of about 3 bar to about 10 bar.

71. The method of claim 57, wherein step a) is performed in an alcohol solvent.

72. The method of claim 71, wherein the solvent is methanol, ethanol, or isopropanol.

73. The method of claim 57, wherein (R)-6-hydroxychromane-3-carboxylic acid and (S)-6-hydroxychromane-3-carboxylic acid of step a) has an enantiomeric excess of at least 90%.

74. The method of claim 57, wherein (R)-6-((7-oxo-5,6,7,8-tetrahydro-1,8-naphthyridin-4-yl)oxy) chromane-3-carboxylic acid and (S)-6-((7-oxo-5,6,7,8-tetrahydro-1,8-naphthyridin-4-yl)oxy) chromane-3-carboxylic acid of step b) has an enantiomeric excess of at least 90%.

75. The method of claim 57, wherein (R)—N-[2-(4-fluorophenyl)-2-oxo-ethyl]-6-[(7-oxo-6,8-dihydro-5H-1,8-naphthyridin-4-yl)oxy]chromane-3-carboxamide and (S)—N-[2-(4-fluorophenyl)-2-oxo-ethyl]-6-[(7-oxo-6,8-dihydro-5H-1,8-naphthyridin-4-yl)oxy]chromane-3-carboxamide of step c) has an enantiomeric excess of at least 90%.

76. The method of claim 57, wherein (R)-5-[3-[4-(4-fluorophenyl)-1H-imidazol-2-yl]chroman-6-yl]oxy-3,4-dihydro-1H-1,8-naphthyridin-2-one and (S)-5-[3-[4-(4-fluorophenyl)-1H-imidazol-2-yl]chroman-6-yl]oxy-3,4-dihydro-1H-1,8-naphthyridin-2-one, or a pharmaceutically acceptable salt or tautomer thereof, has an enantiomeric excess of at least 90%.

77. A compound having the structure 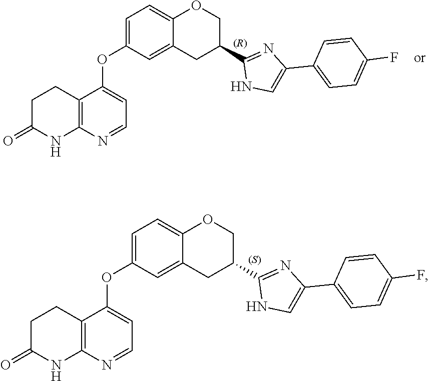
or a pharmaceutically acceptable salt or tautomer thereof, prepared by the method of claim 57.

78. A method of treating a condition which is modulated by a RAF kinase, comprising administering an effective amount of the compound of claim 77 to a subject in need thereof.
